(19) United States
(12) Patent Application Publication
(10) Pub. No.: US 2024/0400562 A1
(43) Pub. Date: Dec. 5, 2024

(54) COMPOUNDS AND COMPOSITIONS FOR TREATING CONDITIONS ASSOCIATED WITH LPA RECEPTOR ACTIVITY

(71) Applicant: LHOTSE, BIO, INC., South San Francisco, CA (US)

(72) Inventors: Hui LEI, Shanghai (CN); Qiong ZHANG, Shanghai (CN); Anatoly M. Ruvinsky, South San Francisco, CA (US)

(21) Appl. No.: 18/701,565

(22) PCT Filed: Oct. 21, 2022
(86) PCT No.: PCT/CN2022/126596

§ 371 Date: Apr. 15, 2024

(30) Foreign Application Priority Data
Oct. 21, 2021 (WO) PCT/CN2021/125409
Mar. 21, 2022 (WO) PCT/CN2022/082072

(51) Int. Cl. C07D 471/04; A61K 31/426; A61K 31/428; A61K 31/437; C07D 277/24; C07D 277/64

(52) U.S. Cl. CPC C07D 471/04; A61K 31/426; A61K 31/428; A61K 31/437; C07D 277/24; C07D 277/64

(57) ABSTRACT

It provides LPA antagonists, as well as pharmaceutical compositions comprising the compounds disclosed herein. It also provides methods for treating LPA-associated diseases, disorders, and conditions.

### CROSS REFERENCE TO RELATED APPLICATIONS

The application claims the benefit of International Patent Application Number PCT/CN2022/082072, filed on Mar. 21, 2022, and International Patent Application Number PCT/CN2021/125409, filed Oct. 21, 2021, each of which is incorporated herein by reference in its entirety.

### FIELD

The present disclosure provides LPA antagonists, as well as pharmaceutical compositions comprising the compounds disclosed herein. Also provided are methods for treating LPA-associated diseases, disorders, and conditions.

### BACKGROUND

Various lipid mediators, including eicosanoid and platelet activating factor (PAF) are produced by the activity of phospholipase from cell membranes. Lysophospholipids are one class of these membrane-derived bioactive lipid mediators and include lysophosphatidic acid (LPA). LPA is not a single molecular entity but a collection of endogenous structural variants with fatty acids of varied lengths and degrees of saturation. LPAs affect cellular functions that include cellular proliferation, differentiation, survival, migration, adhesion, invasion, and morphogenesis. These functions influence many biological processes that include neurogenesis, angiogenesis, wound healing, immunity, and carcinogenesis. LPA has a role as a biological effector molecule and has a diverse range of physiological actions such as, but not limited to, effects on blood pressure, platelet activation, and smooth muscle contraction, and a variety of cellular effects, which include cell growth, cell rounding, neurite retraction, and actin stress fiber formation and cell migration. The effects of LPA are predominantly receptor mediated. Activation of the LPA receptors (LPA₁, LPA₂, LPA₃, LPA₄, LPA₅, LPA₆) with LPA mediates a range of downstream signaling cascades.

### SUMMARY

Antagonizing LPA receptors (such as the LPA₁ receptor) may be useful for the treatment of a variety of disorders, including fibrosis such as pulmonary fibrosis, hepatic fibrosis, renal fibrosis, arterial fibrosis and systemic sclerosis, and thus the diseases that result from fibrosis (e.g., pulmonary fibrosis, for example, Idiopathic Pulmonary Fibrosis (IPF), hepatic fibrosis, including Non-alcoholic Steatohepatitis (NASH), renal fibrosis, such as diabetic nephropathy, systemic sclerosis-scleroderma, etc.), COVID-19, chronic obstructive pulmonary disease (COPD), neuroinflammation, or multiple sclerosis. The present application describes LPA antagonists, as well as pharmaceutical compositions comprising the compounds disclosed herein. Also provided are methods for treating LPA-associated diseases, disorders, and conditions.

In one embodiment is provided the compounds of Table 1, or a pharmaceutically acceptable salt or solvate thereof. In one embodiment, is provided the compounds of Table 2, or a pharmaceutically acceptable salt, solvate, stereoisomer, or mixture of stereoisomers thereof.

Also provided herein are pharmaceutical compositions comprising a compound of Table 1, or a pharmaceutically acceptable salt or solvate thereof, and a pharmaceutically acceptable excipient.

Also provided herein are methods for treating or preventing an LPA-associated disease in a subject in need thereof, the method comprising administering to subject a therapeutically effective amount of a compound disclosed herein (e.g., a compound of Table 1 or Table 2, or a pharmaceutically acceptable salt or solvate thereof), or a pharmaceutical composition thereof. In some embodiments, the LPA-associated disease is an LPA₁-associated disease, such as, but not limited to, fibrosis, transplant rejection, cancer, osteoporosis, or an inflammatory disorder.

In some embodiments, the LPA-associated disease is fibrosis, transplant rejection, cancer, osteoporosis, or inflammatory disorders. In certain of these embodiments, the fibrosis is pulmonary, liver, renal, cardiac, dermal, ocular, or pancreatic fibrosis. In certain embodiments, the cancer is of the bladder, blood, bone, brain, breast, central nervous system, cervix, colon, endometrium, esophagus, gall bladder, genitalia, genitourinary tract, head, kidney, larynx, liver, lung, muscle tissue, neck, oral or nasal mucosa, ovary, pancreas, prostate, skin, spleen, small intestine, large intestine, stomach, testicle, or thyroid.

In some embodiments, the LPA-associated disease is idiopathic pulmonary fibrosis (IPF), non-alcoholic steatohepatitis (NASH), non-alcoholic fatty liver disease (NAFLD), chronic kidney disease, diabetic kidney disease, systemic sclerosis, COVID-19, chronic obstructive pulmonary disease (COPD), neuroinflammation, or multiple sclerosis.

Also provided herein are methods for treating or preventing fibrosis in a subject in need thereof, the method comprising administering to subject a therapeutically effective amount of a compound disclosed herein (e.g., a compound of Table 1 or Table 2, or a pharmaceutically acceptable salt or solvate thereof), or a pharmaceutically acceptable salt or solvate thereof, or a pharmaceutical composition thereof.

In some embodiments, the fibrosis is idiopathic pulmonary fibrosis (IPF), nonalcoholic steatohepatitis (NASH), chronic kidney disease, diabetic kidney disease, and systemic sclerosis. For example, the fibrosis can be IPF.

### DETAILED DESCRIPTION

### Definitions

The following description sets forth exemplary embodiments of the present technology. It should be recognized, however, that such description is not intended as a limitation on the scope of the present disclosure but is instead provided as a description of exemplary embodiments.

As used in the present specification, the following words, phrases and symbols are generally intended to have the meanings as set forth below, except to the extent that the context in which they are used indicates otherwise.

As used herein, the term “compound,” is meant to include all stereoisomers, geometric isomers, tautomers, and isotopes of the structures depicted. Compounds herein identified by name or structure as one particular tautomeric form are intended to include other tautomeric forms unless otherwise specified.

Some of the compounds exist as tautomers. Tautomers are in equilibrium with one another. For example, amide containing compounds may exist in equilibrium with imidic acid tautomers. Regardless of which tautomer is shown, and regardless of the nature of the equilibrium among tautomers, the compounds are understood by one of ordinary skill in the art to comprise both amide and imidic acid tautomers. Thus, the amide containing compounds are understood to include their imidic acid tautomers. Likewise, the imidic acid containing compounds are understood to include their amide tautomers.

Any compound or structure given herein, is also intended to represent unlabeled forms as well as isotopically labeled forms of the compounds. These forms of compounds may also be referred to as “isotopically enriched analogs.” Isotopically labeled compounds have structures depicted herein, except that one or more atoms are replaced by an atom having a selected atomic mass or mass number. Examples of isotopes that can be incorporated into the disclosed compounds include isotopes of hydrogen, carbon, nitrogen, oxygen, phosphorous, fluorine, chlorine, and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. Various isotopically labeled compounds of the present disclosure, for example those into which radioactive isotopes such as ³H and ¹⁴C are incorporated. Such isotopically labelled compounds may be useful in metabolic studies, reaction kinetic studies, detection or imaging techniques, such as positron emission tomography (PET) or single-photon emission computed tomography (SPECT) including drug or substrate tissue distribution assays or in radioactive treatment of patients.

The term “isotopically enriched analogs” includes “deuterated analogs” of compounds described herein in which one or more hydrogens is/are replaced by deuterium, such as a hydrogen on a carbon atom. Such compounds exhibit increased resistance to metabolism and are thus useful for increasing the half-life of any compound when administered to a mammal, particularly a human. See, for example, Foster. “Deuterium Isotope Effects in Studies of Drug Metabolism,” Trends Pharmacol. Sci. 5(12):524-527 (1984). Such compounds are synthesized by means well known in the art, for example by employing starting materials in which one or more hydrogens have been replaced by deuterium.

Deuterium labelled or substituted therapeutic compounds of the disclosure may have improved DMPK (drug metabolism and pharmacokinetics) properties, relating to distribution, metabolism, and excretion (ADME). Substitution with heavier isotopes such as deuterium may afford certain therapeutic advantages resulting from greater metabolic stability, for example increased in vivo half-life, reduced dosage requirements, and/or an improvement in therapeutic index. An ¹⁸F, ³H, ¹¹C labeled compound may be useful for PET or SPECT or other imaging studies. Isotopically labeled compounds of this disclosure and prodrugs thereof can generally be prepared by carrying out the procedures disclosed in the schemes or in the examples and preparations described below by substituting a readily available isotopically labeled reagent for a non-isotopically labeled reagent. It is understood that deuterium in this context is regarded as a substituent in a compound described herein.

The concentration of such a heavier isotope, specifically deuterium, may be defined by an isotopic enrichment factor. In the compounds of this disclosure any atom not specifically designated as a particular isotope is meant to represent any stable isotope of that atom. Unless otherwise stated, when a position is designated specifically as “H” or “hydrogen,” the position is understood to have hydrogen at its natural abundance isotopic composition. Accordingly, in the compounds of this disclosure any atom specifically designated as a deuterium (D) is meant to represent deuterium.

In many cases, the compounds of this disclosure are capable of forming acid and/or base salts by virtue of the presence of amino and/or carboxyl groups or groups similar thereto.

Provided are also pharmaceutically acceptable salts, hydrates, solvates, tautomeric forms, polymorphs, and prodrugs of the compounds described herein. “Pharmaceutically acceptable” or “physiologically acceptable” refer to compounds, salts, compositions, dosage forms and other materials which are useful in preparing a pharmaceutical composition that is suitable for veterinary or human pharmaceutical use.

The term “pharmaceutically acceptable salt” of a given compound refers to salts that retain the biological effectiveness and properties of the given compound and which are not biologically or otherwise undesirable. “Pharmaceutically acceptable salts” or “physiologically acceptable salts” include, for example, salts with inorganic acids and salts with an organic acid. In addition, if the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare nontoxic pharmaceutically acceptable addition salts. Pharmaceutically acceptable acid addition salts may be prepared from inorganic and organic acids. Salts derived from inorganic acids include, e.g., hydrochloric acid, hydrobromic acid, sulfuric acid, nitric acid, phosphoric acid and the like. Salts derived from organic acids include, e.g., acetic acid, propionic acid, gluconic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like. Likewise, pharmaceutically acceptable base addition salts can be prepared from inorganic and organic bases. Salts derived from inorganic bases include, by way of example only, sodium, potassium, lithium, aluminum, ammonium, calcium, and magnesium salts. Salts derived from organic bases include, but are not limited to, salts of NH₃, or primary, secondary, tertiary amines, such as salts derived from a N-containing heterocycle, a N-containing heteroaryl, or derived from an amine of formula N(R^{N})₃ (e.g., HN⁺(R^{N})₃ or (alkyl)N⁺(R^{N})₃) where each R^{N} is independently hydrogen, alkyl, alkenyl, alkynyl, haloalkyl, cycloalkyl, heterocyclyl, aryl, or heteroaryl, wherein each is optionally substituted, such as by one or more (e.g., 1-5 or 1-3) substituents (e.g., halo, cyano, hydroxy, amino, alkyl, alkenyl, alkynyl, haloalkyl, alkoxy, or haloalkoxy). Specific examples of suitable amines include, by way of example only, isopropylamine, trimethyl amine, diethyl amine, tri(isopropyl) amine, tri(n-propyl) amine, ethanolamine, 2-dimethylaminoethanol, piperazine, piperidine, morpholine, N-ethylpiperidine, and the like.

The term “substituted” means that any one or more hydrogen atoms on the designated atom or group is replaced with one or more substituents other than hydrogen, provided that the designated atom's normal valence is not exceeded. The one or more substituents include, but are not limited to, alkyl, alkenyl, alkynyl, alkoxy, acyl, amino, amido, amidino, aryl, azido, carbamoyl, carboxyl, carboxyl ester, cyano, guanidino, halo, haloalkyl, haloalkoxy, heteroalkyl, heteroaryl, heterocyclyl, hydroxy, hydrazino, imino, oxo, nitro, alkylsulfinyl, sulfonic acid, alkylsulfonyl, thiocyanate, thiol, thione, or combinations thereof.

Polymers or similar indefinite structures arrived at by defining substituents with further substituents appended ad infinitum (e.g., a substituted aryl having a substituted alkyl which is itself substituted with a substituted aryl group, which is further substituted by a substituted heteroalkyl group, etc.) are not intended for inclusion herein. Unless otherwise noted, the maximum number of serial substitutions in compounds described herein is three. For example, serial substitutions of substituted aryl groups with two other substituted aryl groups are limited to ((substituted aryl)substituted aryl) substituted aryl. Similarly, the above definitions are not intended to include impermissible substitution patterns (e.g., methyl substituted with 5 fluorines or heteroaryl groups having two adjacent oxygen ring atoms). Such impermissible substitution patterns are well known to the skilled artisan. When used to modify a chemical group, the term “substituted” may describe other chemical groups defined herein. Unless specified otherwise, where a group is described as optionally substituted, any substituents of the group are themselves unsubstituted. For example, in some embodiments, the term “substituted alkyl” refers to an alkyl group having one or more substituents including hydroxyl, halo, alkoxy, cycloalkyl, heterocyclyl, aryl, and heteroaryl. In other embodiments, the one or more substituents may be further substituted with halo, alkyl, haloalkyl, hydroxyl, alkoxy, cycloalkyl, heterocyclyl, aryl, or heteroaryl, each of which is substituted. In other embodiments, the substituents may be further substituted with halo, alkyl, haloalkyl, alkoxy, hydroxyl, cycloalkyl, heterocyclyl, aryl, or heteroaryl, each of which is unsubstituted.

The terms “optional” or “optionally” means that the subsequently described event or circumstance may or may not occur, and that the description includes instances where said event or circumstance occurs and instances in which it does not. Also, the term “optionally substituted” refers to any one or more hydrogen atoms on the designated atom or group may or may not be replaced by a moiety other than hydrogen.

A dash (“—”) that is not between two letters or symbols is used to indicate a point of attachment for a substituent. For example, —C(O)NH₂ is attached through the carbon atom. A dash at the front or end of a chemical group is a matter of convenience; chemical groups may be depicted with or without one or more dashes without losing their ordinary meaning. A wavy line drawn through a line in a structure indicates a point of attachment of a group. Unless chemically or structurally required, no directionality is indicated or implied by the order in which a chemical group is written or named.

The prefix “Cᵤ₋ᵥ” indicates that the following group has from u to v carbon atoms. For example, “C₁₋₆ alkyl” indicates that the alkyl group has from 1 to 6 carbon atoms.

Reference to “about” a value or parameter herein includes (and describes) embodiments that are directed to that value or parameter per se. In certain embodiments, the term “about” includes the indicated amount 10%. In other embodiments, the term “about” includes the indicated amount ±5%. In certain other embodiments, the term “about” includes the indicated amount ±1%. Also, to the term “about X” includes description of “X”. Also, the singular forms “a” and “the” include plural references unless the context clearly dictates otherwise. Thus, e.g., reference to “the compound” includes a plurality of such compounds and reference to “the assay” includes reference to one or more assays and equivalents thereof known to those skilled in the art.

“Alkyl” refers to an unbranched or branched saturated hydrocarbon chain. As used herein, alkyl has 1 to 20 carbon atoms (i.e., C₁₋₂₀ alkyl), 1 to 12 carbon atoms (i.e., C₁₋₁₂ alkyl), 1 to 8 carbon atoms (i.e., C₁₋₈ alkyl), 1 to 6 carbon atoms (i.e., C₁₋₆ alkyl), or 1 to 4 carbon atoms (i.e., C₁₋₄ alkyl). Examples of alkyl groups include, e.g., methyl, ethyl, propyl, isopropyl, n-butyl, sec-butyl, iso-butyl, tert-butyl, pentyl, 2-pentyl, isopentyl, neopentyl, hexyl, 2-hexyl, 3-hexyl, and 3-methylpentyl. When an alkyl residue having a specific number of carbons is named by chemical name or identified by molecular formula, all positional isomers having that number of carbons may be encompassed; thus, for example, “butyl” includes n-butyl (i.e., —(CH₂)₃CH₃), sec-butyl (i.e., —CH(CH₃)CH₂CH₃), isobutyl (i.e., —CH₂CH(CH₃)₂), and tert-butyl (i.e., —C(CH₃)₃), and “propyl” includes n-propyl (i.e., —(CH₂)₂CH₃), and isopropyl (i.e., —CH(CH₃)₂).

“Alkenyl” refers to an alkyl group containing at least one (e.g., 1-3, or 1) carbon-carbon double bond and having from 2 to 20 carbon atoms (i.e., C₂₋₂₀ alkenyl), 2 to 12 carbon atoms (i.e., C₂₋₁₂ alkenyl), 2 to 8 carbon atoms (i.e., C₂₋₈ alkenyl), 2 to 6 carbon atoms (i.e., C₂₋₆ alkenyl), or 2 to 4 carbon atoms (i.e., C₂₋₄ alkenyl). Examples of alkenyl groups include, e.g., ethenyl, propenyl, butadienyl (including 1,2-butadienyl, and 1,3-butadienyl).

“Alkynyl” refers to an alkyl group containing at least one (e.g., 1-3, or 1) carbon-carbon triple bond and having from 2 to 20 carbon atoms (i.e., C₂₋₂₀ alkynyl), 2 to 12 carbon atoms (i.e., C₂₋₁₂ alkynyl), 2 to 8 carbon atoms (i.e., C₂₋₈ alkynyl), 2 to 6 carbon atoms (i.e., C₂₋₆ alkynyl), or 2 to 4 carbon atoms (i.e., C₂₋₄ alkynyl). The term “alkynyl” also includes those groups having one triple bond and one double bond.

Certain commonly used alternative chemical names may be used. For example, a divalent group such as a divalent “alkyl” group, a divalent “aryl” group, etc., may also be referred to as an “alkylene” group or an “alkylenyl” group, an “arylene” group or an “arylenyl” group, respectively.

“Alkoxy” refers to the group “alkyl-O—”. Examples of alkoxy groups include, e.g., methoxy, ethoxy, n-propoxy, iso-propoxy, n-butoxy, tert-butoxy, sec-butoxy, n-pentoxy, n-hexoxy, and 1,2-dimethylbutoxy.

“Haloalkyl” refers to an unbranched or branched alkyl group as defined above, wherein one or more (e.g., 1 to 6 or 1 to 3) hydrogen atoms are replaced by a halogen. For example, where a residue is substituted with more than one halogen, it may be referred to by using a prefix corresponding to the number of halogen moieties attached. Dihaloalkyl and trihaloalkyl refer to alkyl substituted with two (“di”) or three (“tri”) halo groups, which may be, but are not necessarily, the same halogen. Examples of haloalkyl include, e.g., trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl, and the like.

“Haloalkoxy” refers to an alkoxy group as defined above, wherein one or more (e.g., 1 to 6 or 1 to 3) hydrogen atoms are replaced by a halogen.

“Hydroxyalkyl” refers to an alkyl group as defined above, wherein one or more (e.g., 1 to 6 or 1 to 3) hydrogen atoms are replaced by a hydroxy group.

“Alkylthio” refers to the group “alkyl-S—”.

“Acyl” refers to a group —C(O)R, wherein R is hydrogen, alkyl, cycloalkyl, heterocyclyl, aryl, heteroalkyl, or heteroaryl; each of which may be optionally substituted, as defined herein. Examples of acyl include formyl, acetyl, cylcohexylcarbonyl, cyclohexylmethyl-carbonyl, and benzoyl.

“Amido” refers to both a “C-amido” group which refers to the group —C(O)NR^{y}R^{z} and an “N-amido” group which refers to the group —NR^{y}C(O)R^{z}, wherein R^{y} and R^{z} are independently hydrogen, alkyl, alkenyl, alkynyl, cycloalkyl, heterocyclyl, aryl, heteroalkyl, or heteroaryl; each of which may be optionally substituted, as defined herein, or R^{y} and R^{z} are taken together to form a cycloalkyl or heterocyclyl; each of which may be optionally substituted, as defined herein.

“Amino” refers to the group —NR^{y}R^{z} wherein R^{y} and R^{z} are independently hydrogen, alkyl, alkenyl, alkynyl, cycloalkyl, heterocyclyl, aryl, heteroalkyl, or heteroaryl; each of which may be optionally substituted, as defined herein.

“Amidino” refers to —C(NR^{y})(NR^{z}₂), wherein R^{y} and R^{z} are independently hydrogen, alkyl, alkenyl, alkynyl, cycloalkyl, heterocyclyl, aryl, heteroalkyl, or heteroaryl; each of which may be optionally substituted, as defined herein.

“Aryl” refers to an aromatic carbocyclic group having a single ring (e.g., monocyclic) or multiple rings (e.g., bicyclic or tricyclic) including fused systems. As used herein, aryl has 6 to 20 ring carbon atoms (i.e., C₆₋₂₀ aryl), 6 to 12 carbon ring atoms (i.e., C₆₋₁₂ aryl), or 6 to 10 carbon ring atoms (i.e., C₆₋₁₀ aryl). Examples of aryl groups include, e.g., phenyl, naphthyl, fluorenyl, and anthryl. Aryl, however, does not encompass or overlap in any way with heteroaryl defined below. If one or more aryl groups are fused with a heteroaryl, the resulting ring system is heteroaryl regardless of point of attachment. If one or more aryl groups are fused with a heterocyclyl, the resulting ring system is heterocyclyl regardless of point of attachment. If one or more aryl groups are fused with a cycloalkyl, the resulting ring system is cycloalkyl regardless of point of attachment.

“Carbamoyl” refers to both an “O-carbamoyl” group which refers to the group —O—C(O)NR^{y}R^{z} and an “N-carbamoyl” group which refers to the group —NR^{y}C(O)OR^{z}, wherein R^{y} and R^{z} are independently hydrogen, alkyl, alkenyl, alkynyl, cycloalkyl, heterocyclyl, aryl, heteroalkyl, or heteroaryl; each of which may be optionally substituted, as defined herein.

“Carboxyl ester” or “ester” refer to both —OC(O)R^{x} and —C(O)OR^{x}, wherein R^{x} is alkyl, alkenyl, alkynyl, cycloalkyl, heterocyclyl, aryl, heteroalkyl, or heteroaryl; each of which may be optionally substituted, as defined herein.

“Cycloalkyl” refers to a saturated or partially unsaturated cyclic alkyl group having a single ring or multiple rings including fused, bridged, and spiro ring systems. The term “cycloalkyl” includes cycloalkenyl groups (i.e., the cyclic group having at least one double bond) and carbocyclic fused ring systems having at least one sp³ carbon atom (i.e., at least one non-aromatic ring). As used herein, cycloalkyl has from 3 to 20 ring carbon atoms (i.e., C₃₋₂₀ cycloalkyl), 3 to 14 ring carbon atoms (i.e., C₃₋₁₂ cycloalkyl), 3 to 12 ring carbon atoms (i.e., C₃₋₁₂ cycloalkyl), 3 to 10 ring carbon atoms (i.e., C₃₋₁₀ cycloalkyl), 3 to 8 ring carbon atoms (i.e., C₃₋₈ cycloalkyl), or 3 to 6 ring carbon atoms (i.e., C₃₋₆ cycloalkyl). Monocyclic groups include, for example, cyclopropyl, cyclobutyl, cyclopentyl, cyclohexyl, cycloheptyl, and cyclooctyl. Polycyclic groups include, for example, bicyclo[2.2.1]heptanyl, bicyclo[2.2.2]octanyl, adamantyl, norbornyl, decalinyl, 7,7-dimethyl-bicyclo[2.2.1]heptanyl, and the like. Further, the term cycloalkyl is intended to encompass any non-aromatic ring which may be fused to an aryl ring, regardless of the attachment to the remainder of the molecule. Still further, cycloalkyl also includes “spirocycloalkyl” when there are two positions for substitution on the same carbon atom, for example spiro[2.5]octanyl, spiro[4.5]decanyl, or spiro[5.5]undecanyl.

“Imino” refers to a group —C(NR^{y})R^{z}, wherein R^{y} and R^{z} are each independently hydrogen, alkyl, alkenyl, alkynyl, cycloalkyl, heterocyclyl, aryl, heteroalkyl, or heteroaryl; each of which may be optionally substituted, as defined herein.

“Halogen” or “halo” refers to atoms occupying group VIIA of the periodic table, such as fluoro, chloro, bromo, or iodo.

“Heteroalkyl” refers to an alkyl group in which one or more of the carbon atoms (and any associated hydrogen atoms) are each independently replaced with the same or different heteroatomic group. The term “heteroalkyl” includes unbranched or branched saturated chain having carbon and heteroatoms. By way of example, 1, 2 or 3 carbon atoms may be independently replaced with the same or different heteroatomic group. Heteroatomic groups include, but are not limited to, —NR—, —O—, —S—, —S(O)—, —S(O)₂—, and the like, where R is H, alkyl, aryl, cycloalkyl, heteroalkyl, heteroaryl or heterocyclyl, each of which may be optionally substituted. Examples of heteroalkyl groups include —OCH₃, —CH₂OCH₃, —SCH₃, —CH₂SCH₃, —NRCH₃, and —CH₂NRCH₃, where R is hydrogen, alkyl, aryl, arylalkyl, heteroalkyl, or heteroaryl, each of which may be optionally substituted. As used herein, heteroalkyl include 1 to 10 carbon atoms, 1 to 8 carbon atoms, or 1 to 4 carbon atoms; and 1 to 3 heteroatoms, 1 to 2 heteroatoms, or 1 heteroatom.

“Heteroaryl” refers to an aromatic group having a single ring or multiple fused rings, with one or more ring heteroatoms independently selected from nitrogen, oxygen, and sulfur. As used herein, heteroaryl includes 1 to 20 ring carbon atoms (i.e., C₁₋₂₀ heteroaryl), 3 to 12 ring carbon atoms (i.e., C₃₋₁₂ heteroaryl), or 3 to 8 carbon ring atoms (i.e., C₃₋₈ heteroaryl), and 1 to 5 ring heteroatoms, 1 to 4 ring heteroatoms, 1 to 3 ring heteroatoms, 1 to 2 ring heteroatoms, or 1 ring heteroatom independently selected from nitrogen, oxygen, and sulfur. In certain instances, heteroaryl includes 5-10 membered ring systems, 5-7 membered ring systems, or 5-6 membered ring systems, each independently having 1 to 4 ring heteroatoms, 1 to 3 ring heteroatoms, 1 to 2 ring heteroatoms, or 1 ring heteroatom independently selected from nitrogen, oxygen, and sulfur. Examples of heteroaryl groups include, e.g., acridinyl, benzimidazolyl, benzothiazolyl, benzindolyl, benzofuranyl, benzothiazolyl, benzothiadiazolyl, benzonaphthofuranyl, benzoxazolyl, benzothienyl (benzothiophenyl), benzotriazolyl, benzo[4,6]imidazo[1,2-a]pyridyl, carbazolyl, cinnolinyl, dibenzofuranyl, dibenzothiophenyl, furanyl, isothiazolyl, imidazolyl, indazolyl, indolyl, indazolyl, isoindolyl, isoquinolyl, isoxazolyl, naphthyridinyl, oxadiazolyl, oxazolyl, 1-oxidopyridinyl, 1-oxidopyrimidinyl, 1-oxidopyrazinyl, 1-oxidopyridazinyl, phenazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, thiazolyl, thiadiazolyl, triazolyl, tetrazolyl, and triazinyl. Examples of the fused-heteroaryl rings include, but are not limited to, benzo[d]thiazolyl, quinolinyl, isoquinolinyl, benzo[b]thiophenyl, indazolyl, benzo[d]imidazolyl, pyrazolo[1,5-a]pyridinyl, and imidazo[1,5-a]pyridinyl, where the heteroaryl can be bound via either ring of the fused system. Any aromatic ring, having a single or multiple fused rings, containing at least one heteroatom, is considered a heteroaryl regardless of the attachment to the remainder of the molecule (i.e., through any one of the fused rings). Heteroaryl does not encompass or overlap with aryl as defined above.

“Heterocyclyl” refers to a saturated or partially unsaturated cyclic alkyl group, with one or more ring heteroatoms independently selected from nitrogen, oxygen, and sulfur. The term “heterocyclyl” includes heterocycloalkenyl groups (i.e., the heterocyclyl group having at least one double bond), bridged-heterocyclyl groups, fused-heterocyclyl groups, and spiro-heterocyclyl groups. A heterocyclyl may be a single ring or multiple rings wherein the multiple rings may be fused, bridged, or spiro, and may comprise one or more (e.g., 1 to 3) oxo (=O) or N-oxide (—O⁻) moieties. Any non-aromatic ring containing at least one heteroatom is considered a heterocyclyl, regardless of the attachment (i.e., can be bound through a carbon atom or a heteroatom). Further, the term heterocyclyl is intended to encompass any non-aromatic ring containing at least one heteroatom, which ring may be fused to a cycloalkyl, an aryl, or heteroaryl ring, regardless of the attachment to the remainder of the molecule. As used herein, heterocyclyl has 2 to 20 ring carbon atoms (i.e., C₂₋₂₀ heterocyclyl), 2 to 12 ring carbon atoms (i.e., C₂₋₁₂ heterocyclyl), 2 to 10 ring carbon atoms (i.e., C₂₋₁₀ heterocyclyl), 2 to 8 ring carbon atoms (i.e., C₂₋₈ heterocyclyl), 3 to 12 ring carbon atoms (i.e., C₃₋₁₂ heterocyclyl), 3 to 8 ring carbon atoms (i.e., C₃₋₈ heterocyclyl), or 3 to 6 ring carbon atoms (i.e., C₃₋₆ heterocyclyl); having 1 to 5 ring heteroatoms, 1 to 4 ring heteroatoms, 1 to 3 ring heteroatoms, 1 to 2 ring heteroatoms, or 1 ring heteroatom independently selected from nitrogen, sulfur, or oxygen. Examples of heterocyclyl groups include, e.g., azetidinyl, azepinyl, benzodioxolyl, benzo[b][1,4]dioxepinyl, 1,4-benzodioxanyl, benzopyranyl, benzodioxinyl, benzopyranonyl, benzofuranonyl, dioxolanyl, dihydropyranyl, hydropyranyl, thienyl[1,3]dithianyl, decahydroisoquinolyl, furanonyl, imidazolinyl, imidazolidinyl, indolinyl, indolizinyl, isoindolinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, oxiranyl, oxetanyl, phenothiazinyl, phenoxazinyl, piperidinyl, piperazinyl, 4-piperidonyl, pyrrolidinyl, pyrazolidinyl, quinuclidinyl, thiazolidinyl, tetrahydrofuryl, tetrahydropyranyl, trithianyl, tetrahydroquinolinyl, thiophenyl (i.e., thienyl), thiomorpholinyl, thiamorpholinyl, 1-oxo-thiomorpholinyl, and 1,1-dioxo-thiomorpholinyl. The term “heterocyclyl” also includes “spiroheterocyclyl” when there are two positions for substitution on the same carbon atom. Examples of the spiro-heterocyclyl rings include, e.g., bicyclic and tricyclic ring systems, such as oxabicyclo[2.2.2]octanyl, 2-oxa-7-azaspiro[3.5]nonanyl, 2-oxa-6-azaspiro[3.4]octanyl, and 6-oxa-1-azaspiro[3.3]heptanyl. Examples of the fused-heterocyclyl rings include, but are not limited to, 1,2,3,4-tetrahydroisoquinolinyl, 4,5,6,7-tetrahydrothieno[2,3-c]pyridinyl, indolinyl, and isoindolinyl, where the heterocyclyl can be bound via either ring of the fused system.

“Sulfonyl” refers to the group —S(O)₂R^{y}, where R^{y} is hydrogen, alkyl, alkenyl, alkynyl, cycloalkyl, heterocyclyl, aryl, heteroalkyl, or heteroaryl; each of which may be optionally substituted, as defined herein. Examples of sulfonyl are methylsulfonyl, ethylsulfonyl, phenylsulfonyl, and toluenesulfonyl.

“Alkylsulfonyl” refers to the group —S(O)₂R, where R is alkyl.

“Alkylsulfinyl” refers to the group —S(O)R, where R is alkyl.

As used herein, “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

A “solvate” is formed by the interaction of a solvent and a compound. Solvates of salts of the compounds described herein are also provided. Hydrates of the compounds described herein are also provided.

The term “LPA-associated disease” as used herein is meant to include, without limitation, those diseases, disorders, or conditions in which activation of at least one LPA receptor by LPA contributes to the symptomology or progression of the disease, disorder or condition. These diseases, disorders, or conditions may arise from one or more of a genetic, iatrogenic, immunological, infectious, metabolic, oncological, toxic, surgical, and/or traumatic etiology. Accordingly, inhibiting of one or more lysophosphatidic acid (LPA) receptors (e.g., LPA₁, LPA₂, LPA₃, LPA₄, LPA₅, or LPA₆ receptor) signaling can alter the pathology and/or symptoms and/or progression of the disease, disorder, or condition. In some embodiments, the LPA-associated disease is an LPA1-associated disease, wherein modulating LPA1 receptor signaling can alter the pathology and/or symptoms and/or progression of the disease, disorder, or condition.

The terms “fibrosis” or “fibrosing disorder,” as used herein, refers to conditions that are associated with the abnormal accumulation of cells and/or fibronectin and/or collagen and/or increased fibroblast recruitment and include but are not limited to fibrosis of individual organs or tissues such as the heart, kidney, liver, joints, lung, pleural tissue, peritoneal tissue, skin, cornea, retina, musculoskeletal and digestive tract.

The term “pharmaceutically acceptable” as used herein indicates that the compound, or salt or composition thereof is compatible chemically and/or toxicologically with the other ingredients comprising a formulation and/or the subject being treated therewith.

The term “administration” or “administering” refers to a method of giving a dosage of a compound or pharmaceutical composition to a vertebrate or invertebrate, including a mammal, a bird, a fish, or an amphibian. The method of administration can vary depending on various factors, e.g., the components of the pharmaceutical composition, the site of the disease, and the severity of the disease.

The terms “effective amount” or “effective dosage” or “pharmaceutically effective amount” or “therapeutically effective amount,” as used herein, refer to a sufficient amount of a chemical entity (e.g., a compound of Table 1 or Table 2, or a pharmaceutically acceptable salt or solvate thereof) being administered which will relieve to some extent one or more of the symptoms of the disease or condition being treated, and can include curing the disease. “Curing” means that the symptoms of active disease are eliminated. The result includes reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case is determined using any suitable technique, such as a dose escalation study. In some embodiments, a “therapeutically effective amount” of a compound as provided herein refers to an amount of the compound that is effective as a monotherapy or combination therapy.

The term “excipient” or “pharmaceutically acceptable excipient” means a pharmaceutically-acceptable material, composition, or vehicle, such as a liquid or solid filler, diluent, carrier, solvent, or encapsulating material. In some embodiments, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Remington: The Science and Practice of Pharmacy, 21st ed.; Lippincott Williams & Wilkins: Philadelphia. PA, 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, FL, 2009.

The term “pharmaceutical composition” refers to a mixture of a compound disclosed herein, or a pharmaceutically acceptable salt or solvate thereof as provided herein with other chemical components (referred to collectively herein as “excipients”), such as carriers, stabilizers, diluents, dispersing agents, suspending agents, and/or thickening agents. The pharmaceutical composition facilitates administration of the compound to an organism. Multiple techniques of administering a compound exist in the art including, but not limited to, rectal, oral, intravenous, aerosol, parenteral, ophthalmic, pulmonary, and topical administration.

The terms “treat,” “treating,” and “treatment,” in the context of treating a disease, disorder, or condition, are meant to include alleviating or abrogating a disorder, disease, or condition, or one or more of the symptoms associated with the disorder, disease, or condition; or to slowing the progression, spread or worsening of a disease, disorder or condition or of one or more symptoms thereof.

The term “preventing,” as used herein, is the prevention of the onset, recurrence or spread, in whole or in part, of the disease or condition as described herein, or a symptom thereof.

The terms “subject,” “patient,” or “individual,” as used herein, are used interchangeably and refers to any animal, including mammals such as mice, rats, other rodents, rabbits, dogs, cats, swine, cattle, sheep, horses, primates, and humans. In some embodiments, the term refers to a subject, particularly a mammalian subject, for whom diagnosis, prognosis, or therapy is desired or needed. In some embodiments, the subject is a human. In some embodiments, the subject has experienced and/or exhibited at least one symptom of the disease, disorder, or condition to be treated and/or prevented.

The terms “treatment regimen” and “dosing regimen” are used interchangeably to refer to the dose and timing of administration of each therapeutic agent in a combination.

The term “pharmaceutical combination,” as used herein, refers to a pharmaceutical treatment resulting from the mixing or combining of more than one active ingredient and includes both fixed and non-fixed combinations of the active ingredients.

The term “combination therapy” as used herein refers to a dosing regimen of two different therapeutically active agents (i.e., the components or combination partners of the combination), wherein the therapeutically active agents are administered together or separately in a manner prescribed by a medical care taker or according to a regulatory agency as defined herein.

The term “modulate,” “modulating,” or “modulation,” as used herein, refers to a regulation or an adjustment (e.g., increase or decrease) and can include, for example agonism, partial agonism or antagonism.

### Compounds

Provided herein are compounds that are LPA antagonists. In some embodiments, provided is compound selected from Table 1, or a pharmaceutically acceptable salt or solvate thereof:

TABLE 1

| Compound | Structure |
|---|---|
| 101 | 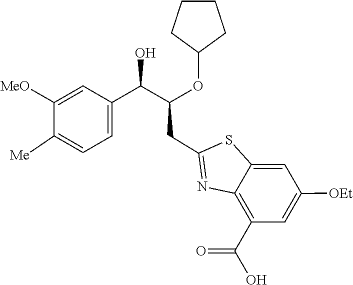 [SMILES: CCOc1cc(C(=O)O)c2nc(C[C@H](OC3CCCC3)[C@H](O)c3ccc(C)c(OC)c3)sc2c1] |
| 102 | 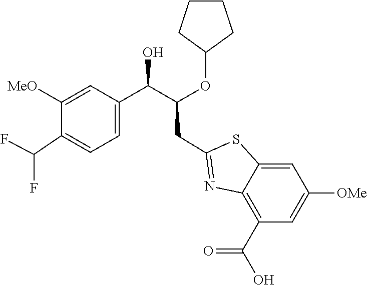 [SMILES: COc1cc(C(=O)O)c2nc(C[C@H](OC3CCCC3)[C@H](O)c3ccc(C(F)F)c(OC)c3)sc2c1] |
| 103 | 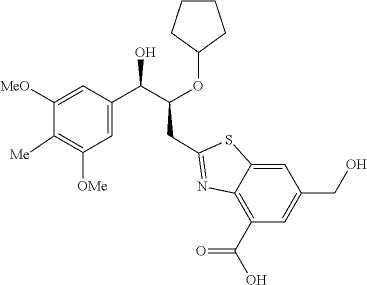 [SMILES: COc1cc([C@@H](O)[C@H](Cc2nc3c(C(=O)O)cc(CO)cc3s2)OC2CCCC2)cc(OC)c1C] |
| 104 | 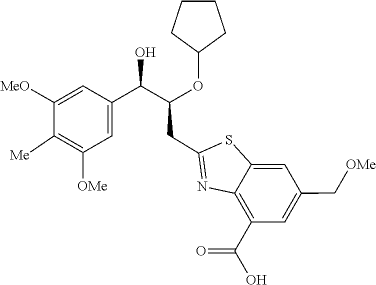 [SMILES: COCc1cc(C(=O)O)c2nc(C[C@H](OC3CCCC3)[C@H](O)c3cc(OC)c(C)c(OC)c3)sc2c1] |
| 105 | 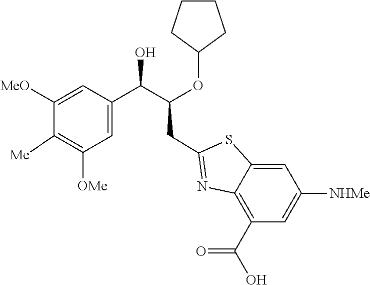 [SMILES: CNc1cc(C(=O)O)c2nc(C[C@H](OC3CCCC3)[C@H](O)c3cc(OC)c(C)c(OC)c3)sc2c1] |
| 106 | 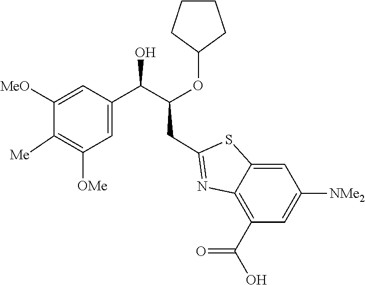 [SMILES: COc1cc([C@@H](O)[C@H](Cc2nc3c(C(=O)O)cc(N(C)C)cc3s2)OC2CCCC2)cc(OC)c1C] |
| 107 | 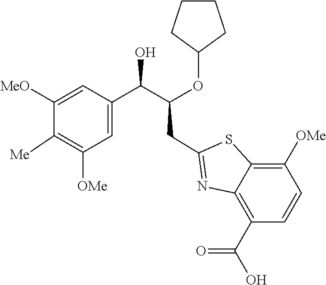 [SMILES: COc1cc([C@@H](O)[C@H](Cc2nc3c(C(=O)O)ccc(OC)c3s2)OC2CCCC2)cc(OC)c1C] |
| 108 | 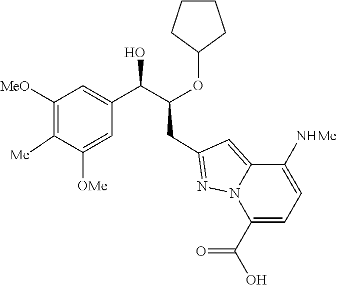 [SMILES: CNc1ccc(C(=O)O)n2nc(C[C@H](OC3CCCC3)[C@H](O)c3cc(OC)c(C)c(OC)c3)cc12] |
| 109 | 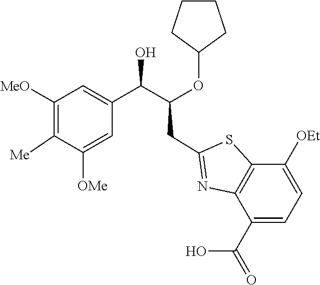 [SMILES: CCOc1ccc(C(=O)O)c2nc(C[C@H](OC3CCCC3)[C@H](O)c3cc(OC)c(C)c(OC)c3)sc12] |
| 110 | 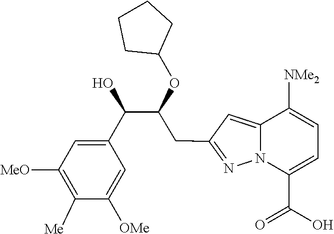 [SMILES: COc1cc([C@@H](O)[C@H](Cc2cc3c(N(C)C)ccc(C(=O)O)n3n2)OC2CCCC2)cc(OC)c1C] |
| 111 | 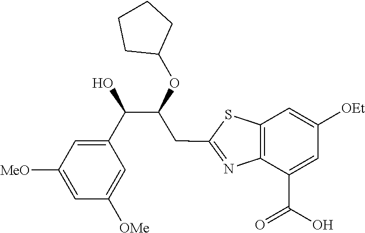 [SMILES: CCOc1cc(C(=O)O)c2nc(C[C@H](OC3CCCC3)[C@H](O)c3cc(OC)cc(OC)c3)sc2c1] |
| 112 | 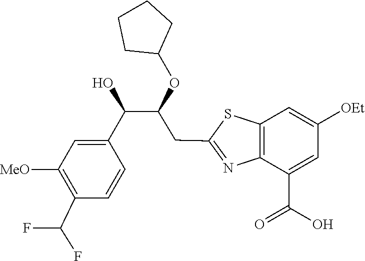 [SMILES: CCOc1cc(C(=O)O)c2nc(C[C@H](OC3CCCC3)[C@H](O)c3ccc(C(F)F)c(OC)c3)sc2c1] |
| 113 | 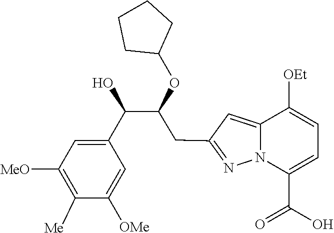 [SMILES: CCOc1ccc(C(=O)O)n2nc(C[C@H](OC3CCCC3)[C@H](O)c3cc(OC)c(C)c(OC)c3)cc12] |
| 114 | 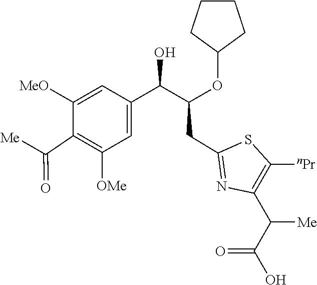 |
| 115 | 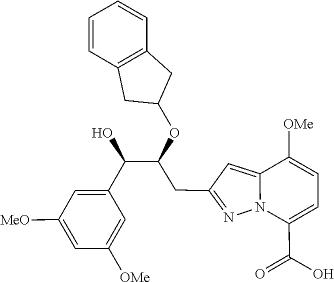 [SMILES: COc1cc(OC)cc([C@@H](O)[C@H](Cc2cc3c(OC)ccc(C(=O)O)n3n2)OC2Cc3ccccc3C2)c1] |
| 116 | 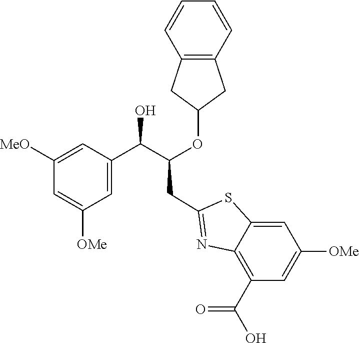 [SMILES: COc1cc(OC)cc([C@@H](O)[C@H](Cc2nc3c(C(=O)O)cc(OC)cc3s2)OC2Cc3ccccc3C2)c1] |
| 117 | 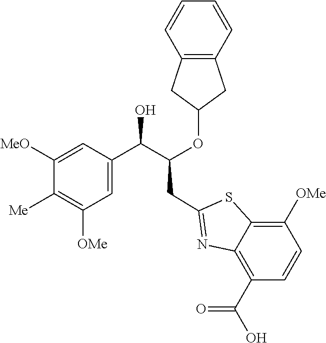 [SMILES: COc1cc([C@@H](O)[C@H](Cc2nc3c(C(=O)O)ccc(OC)c3s2)OC2Cc3ccccc3C2)cc(OC)c1C] |

The compounds provided herein encompass stereochemical forms of the compounds, for example, optical isomers, such as enantiomers, diastereomers, as well as mixtures thereof. e.g., mixtures of enantiomers and/or diastereomers, including racemic mixtures, as well as equal or non-equal mixtures of individual enantiomers and/or diastereomers. Methods known in the art can be used to determine absolute and/or relative configuration, including, but not limited to, chromatography, spectroscopy, X-ray crystallography, and the like. All stereochemical forms are contemplated in this disclosure. Unless otherwise indicated, when a disclosed compound is named or depicted by a structure without specifying the stereochemistry and has one or more chiral centers, it is understood to represent all possible stereoisomers of the compound, such as those delineated in Table 2. In some embodiments, provided is compound selected from Table 2, or a pharmaceutically acceptable salt, solvate, stereoisomer, or mixture of stereoisomers thereof.

TABLE 2
Structure

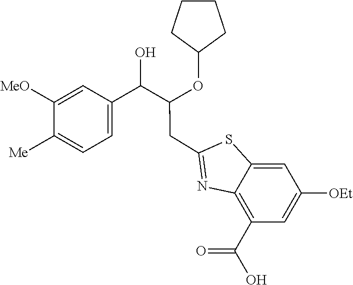 [SMILES: CCOc1cc(C(=O)O)c2nc(CC(OC3CCCC3)C(O)c3ccc(C)c(OC)c3)sc2c1]

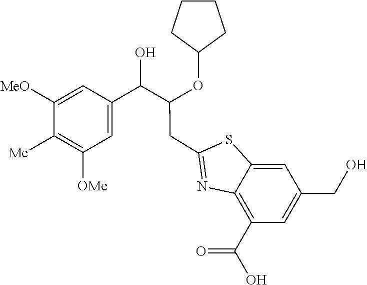 [SMILES: COc1cc(C(O)C(Cc2nc3c(C(=O)O)cc(CO)cc3s2)OC2CCCC2)cc(OC)c1C]

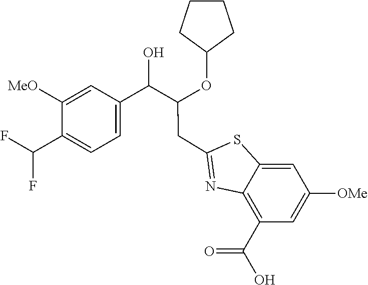 [SMILES: COc1cc(C(=O)O)c2nc(CC(OC3CCCC3)C(O)c3ccc(C(F)F)c(OC)c3)sc2c1]

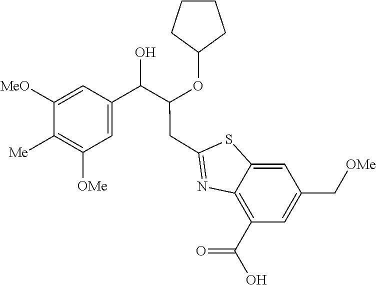 [SMILES: COCc1cc(C(=O)O)c2nc(CC(OC3CCCC3)C(O)c3cc(OC)c(C)c(OC)c3)sc2c1]

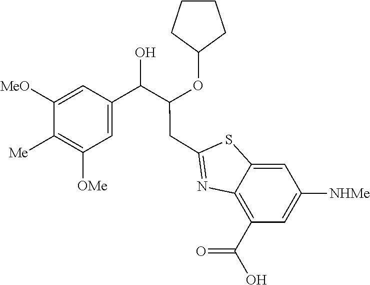 [SMILES: CNc1cc(C(=O)O)c2nc(CC(OC3CCCC3)C(O)c3cc(OC)c(C)c(OC)c3)sc2c1]

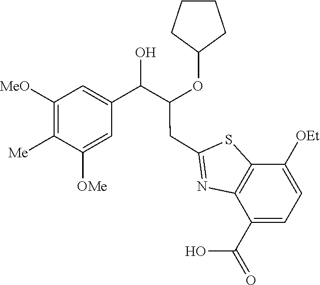 [SMILES: CCOc1ccc(C(=O)O)c2nc(CC(OC3CCCC3)C(O)c3cc(OC)c(C)c(OC)c3)sc12]

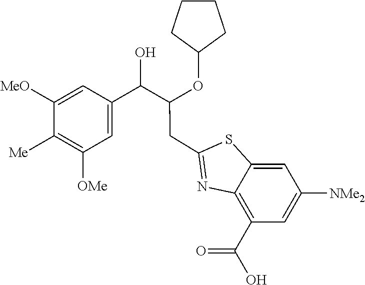 [SMILES: COc1cc(C(O)C(Cc2nc3c(C(=O)O)cc(N(C)C)cc3s2)OC2CCCC2)cc(OC)c1C]

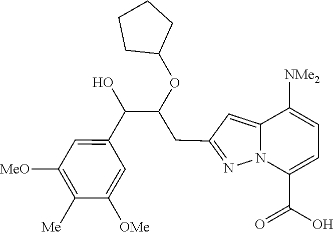 [SMILES: COc1cc(C(O)C(Cc2cc3c(N(C)C)ccc(C(=O)O)n3n2)OC2CCCC2)cc(OC)c1C]

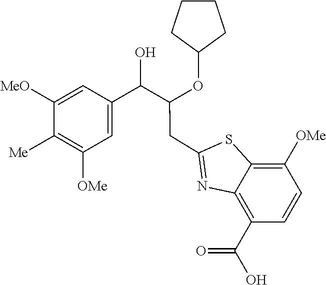 [SMILES: COc1cc(C(O)C(Cc2nc3c(C(=O)O)ccc(OC)c3s2)OC2CCCC2)cc(OC)c1C]

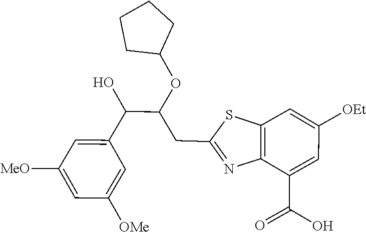 [SMILES: CCOc1cc(C(=O)O)c2nc(CC(OC3CCCC3)C(O)c3cc(OC)cc(OC)c3)sc2c1]

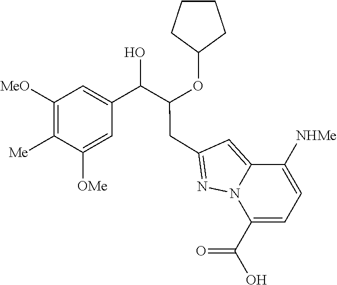 [SMILES: CNc1ccc(C(=O)O)n2nc(CC(OC3CCCC3)C(O)c3cc(OC)c(C)c(OC)c3)cc12]

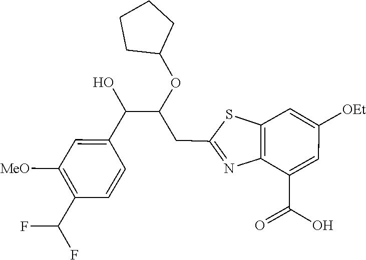 [SMILES: CCOc1cc(C(=O)O)c2nc(CC(OC3CCCC3)C(O)c3ccc(C(F)F)c(OC)c3)sc2c1]

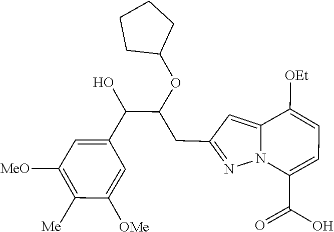 [SMILES: CCOc1ccc(C(=O)O)n2nc(CC(OC3CCCC3)C(O)c3cc(OC)c(C)c(OC)c3)cc12]

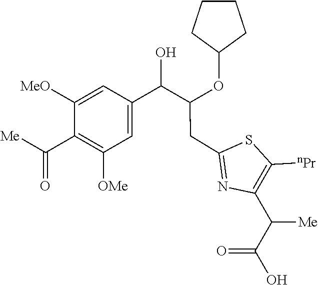

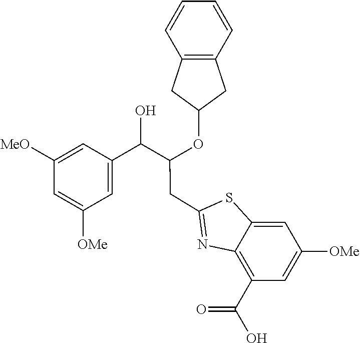 [SMILES: COc1cc(OC)cc(C(O)C(Cc2nc3c(C(=O)O)cc(OC)cc3s2)OC2Cc3ccccc3C2)c1]

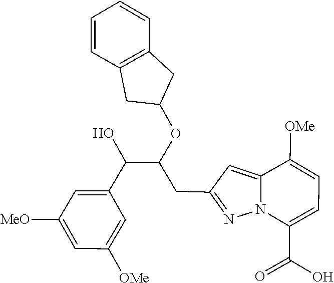 [SMILES: COc1cc(OC)cc(C(O)C(Cc2cc3c(OC)ccc(C(=O)O)n3n2)OC2Cc3ccccc3C2)c1]

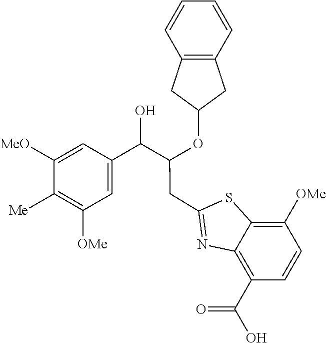 [SMILES: COc1cc(C(O)C(Cc2nc3c(C(=O)O)ccc(OC)c3s2)OC2Cc3ccccc3C2)cc(OC)c1C]

The compounds disclosed herein include pharmaceutically acceptable salts thereof. In addition, the compounds disclosed herein also include other salts of such compounds which are not necessarily pharmaceutically acceptable salts, and which may be useful as intermediates for preparing and/or purifying compounds disclosed herein and/or for separating isomers or enantiomers of the compounds. Non-limiting examples of pharmaceutically acceptable salts of compounds disclosed herein include trifluoroacetic acid salts.

It will further be appreciated that the compounds disclosed herein or their salts may be isolated in the form of solvates, and accordingly that any such solvate is included within the scope of the present disclosure. For example, compounds disclosed herein and salts thereof can exist in unsolvated as well as solvated forms with pharmaceutically acceptable solvents such as water, ethanol, and the like.

### Treatment Methods and Uses

The methods described herein may be applied to cell populations in vivo or ex vivo. “In vivo” means within a living individual, as within an animal or human. In this context, the methods described herein may be used therapeutically in an individual. “Ex vivo” means outside of a living individual. Examples of ex vivo cell populations include in vitro cell cultures and biological samples including fluid or tissue samples obtained from individuals. Such samples may be obtained by methods well known in the art. Exemplary biological fluid samples include blood, cerebrospinal fluid, urine, and saliva. In this context, the compounds and compositions described herein may be used for a variety of purposes, including therapeutic and experimental purposes. For example, the compounds and compositions described herein may be used ex vivo to determine the optimal schedule and/or dosing of administration of a compound of the present disclosure for a given indication, cell type, individual, and other parameters. Information gleaned from such use may be used for experimental purposes or in the clinic to set protocols for in vivo treatment. Other ex vivo uses for which the compounds and compositions described herein may be suited are described below or will become apparent to those skilled in the art. The selected compounds may be further characterized to examine the safety or tolerance dosage in human or non-human subjects. Such properties may be examined using commonly known methods to those skilled in the art.

The compounds as provided herein, or pharmaceutically acceptable salts or solvates thereof, or pharmaceutical compositions of such compounds, are useful as inhibitors of one or more LPA receptors. As described further herein, a compound antagonizing to an LPA receptor can be useful for prevention and/or treatment of diseases such as various kinds of disease including, for example, fibrosis (e.g., renal fibrosis, pulmonary fibrosis, hepatic fibrosis, arterial fibrosis, systemic sclerosis), urinary system disease, carcinoma-associated disease, proliferative disease, inflammation/immune system disease, disease by secretory dysfunction, brain-related disease, and chronic disease.

In some embodiments, this disclosure provides methods for treating a subject (e.g., a human) having a disease, disorder, or condition in which inhibition of one or more LPA receptors (i.e., an LPA-associated disease) is beneficial for the treatment of the underlying pathology and/or symptoms and/or progression of the disease, disorder, or condition. In some embodiments, the methods provided herein can include or further include treating one or more conditions associated, co-morbid or sequela with any one or more of the conditions provided herein.

Provided herein is a method for treating a LPA-associated disease, the method comprising administering to a subject in need thereof an effective amount of a compound disclosed herein (e.g., a compound of Table 1 or Table 2, or a pharmaceutically acceptable salt or solvate thereof), or a pharmaceutical composition as disclosed herein.

In some embodiments, an LPA-associated disease includes, but is not limited to treating fibrosis of an organ (e.g., liver, kidney, lung, heart, and skin), liver disease (acute hepatitis, chronic hepatitis, liver fibrosis, liver cirrhosis, portal hypertension, regenerative failure, non-alcoholic steatohepatitis (NASH), liver hypofunction, hepatic blood flow disorder, and the like), cell proliferative disease (e.g., cancer, including solid tumors, solid tumor metastasis, vascular fibroma, myeloma, multiple myeloma, Kaposi's sarcoma, leukemia, and chronic lymphocytic leukemia (CLL), and invasive metastasis of cancer cells, inflammatory disease (e.g., psoriasis, nephropathy, and pneumonia), gastrointestinal tract disease (e.g., irritable bowel syndrome (TBS), inflammatory bowel disease (IBD), and abnormal pancreatic secretion), renal disease, urinary tract-associated disease (e.g., benign prostatic hyperplasia or symptoms associated with neuropathic bladder disease, spinal cord tumor, hernia of intervertebral disk, spinal canal stenosis, symptoms derived from diabetes, lower urinary tract disease (e.g., obstruction of lower urinary tract), inflammatory disease of the lower urinary tract, dysuna, and frequent urination), pancreas disease, abnormal angiogenesis-associated disease (e.g., arterial obstruction), scleroderma, brain-associated disease (e.g., cerebral infarction and cerebral hemorrhage), neuropathic pain, peripheral neuropathy, ocular disease (e.g., age-related macular degeneration (AMD), diabetic retinopathy, proliferative vitreoretinopathy (PVR), cicatricial pemphigoid, and glaucoma filtration surgery scarring).

In some embodiments, provided herein are methods of treating or preventing fibrosis, the method comprising administering to a subject in need thereof a therapeutically effective amount of a compound disclosed herein (e.g., a compound of Table 1 or Table 2, or a pharmaceutically acceptable salt or solvate thereof), or a pharmaceutical composition as disclosed herein. For example, the methods can include treating renal fibrosis, pulmonary fibrosis, hepatic fibrosis, arterial fibrosis or systemic sclerosis. In some embodiments, provided herein are methods of treating pulmonary fibrosis (e.g., Idiopathic Pulmonary Fibrosis (IPF)), the method comprising administering to a subject in need thereof a therapeutically effective amount of a compound disclosed herein (e.g., a compound of Table 1 or Table 2, or a pharmaceutically acceptable salt or solvate thereof), or a pharmaceutical composition as provided herein.

In some embodiments, a compound disclosed herein (e.g., a compound of Table 1 or Table 2, or a pharmaceutically acceptable salt or solvate thereof), or a pharmaceutical composition as provided herein, is used to treat or prevent fibrosis in a subject. For example, a compound disclosed herein (e.g., a compound of Table 1 or Table 2, or a pharmaceutically acceptable salt or solvate thereof), or a pharmaceutical composition as provided herein, can be used to treat fibrosis of an organ or tissue in a subject. In some embodiments, provided herein is a method for preventing a fibrosis condition in a subject, the method comprising administering to the subject at risk of developing one or more fibrosis conditions a therapeutically effective amount of a compound disclosed herein (e.g., a compound of Table 1 or Table 2, or a pharmaceutically acceptable salt or solvate thereof), or a pharmaceutical composition as provided herein. For example, the subject may have been exposed to one or more environmental conditions that are known to increase the risk of fibrosis of an organ or tissue. In some embodiments, the subject has been exposed to one or more environmental conditions that are known to increase the risk of lung, liver or kidney fibrosis. In some embodiments, the subject has a genetic predisposition of developing fibrosis of an organ or tissue. In some embodiments, a compound disclosed herein (e.g., a compound of Table 1 or Table 2, or a pharmaceutically acceptable salt or solvate thereof), or a pharmaceutical composition as provided herein, is administered to a subject to prevent or minimize scarring following injury. For example, the injury can include surgery.

Exemplary diseases, disorders, or conditions that involve fibrosis include, but are not limited to: lung diseases associated with fibrosis, for example, idiopathic pulmonary fibrosis, iatrogenic drug induced, occupational/environmental induced fibrosis (Farmer lung), granulomatous diseases (sarcoidosis, hypersensitivity pneumonia), collagen vascular disease (scleroderma and others), alveolar proteinosis, langerhans cell granulonmatosis, lymphangioleiomyomatosis, inherited diseases (e.g., Hermansky-Pudlak Syndrome, Tuberous sclerosis, neurofibromatosis, metabolic storage disorders, and familial interstitial lung disease), pulmonary fibrosis secondary to systemic inflammatory disease such as rheumatoid arthritis, scleroderma, lupus, cryptogenic fibrosing alveolitis, radiation induced fibrosis, chronic obstructive pulmonary disease (COPD), scleroderma, bleomycin induced pulmonary fibrosis, chronic asthma, silicosis, asbestos induced pulmonary or pleural fibrosis, acute lung injury, acute respiratory distress syndrome (ARDS), and acute respiratory distress (including bacterial pneumonia induced, trauma induced, viral pneumonia induced, ventilator induced, non-pulmonary sepsis induced, and aspiration induced). Chronic nephropathies associated with injury/fibrosis, kidney fibrosis (renal fibrosis), glomerulonephritis secondary to systemic inflammatory diseases such as lupus and scleroderma, tubulointerstitium fibrosis, glomerular nephritis, glomerular sclerosis, focal segmental, diabetes, glomerular nephritis, focal segmental glomerular sclerosis, IgA nephropathy, hypertension, allograft and Alport Syndrome; dermatological disorders, gut fibrosis, for example, scleroderma, and radiation induced gut fibrosis; liver fibrosis, for example, cirrhosis, alcohol induced liver fibrosis, nonalcoholic steatohepatitis (NASH), non-alcoholic fatty liver disease (NAFLD), toxic/drug induced liver fibrosis (e.g., hemochromatosis), biliary duct injury, primary biliary cirrhosis, infection or viral induced liver fibrosis (e.g., chronic HCV infection), inflammatory/immune disorders, and autoimmune hepatitis; head and neck fibrosis, for example, corneal scarring, e.g., LASIK (laser-assisted in situ keratomileusis), corneal transplant, and trabeculectomy; hypertrophic scarring, Duputren disease, cutaneous fibrosis, cutaneous scleroderma, keloids, e.g., burn induced or surgical; and other fibrotic diseases, e.g., sarcoidosis, scleroderma, spinal cord injury/fibrosis, myelofibrosis, vascular restenosis, atherosclerosis, arteriosclerosis, Wegener's granulomatosis, chronic lymiphocytic leukemia, tumor metastasis, transplant organ rejection (e.g., Bronchiolitis obliterans), endometriosis, neonatal respiratory distress syndrome, and neuropathic pain, fibromyalgia, mixed connective tissue disease, and Peyronie's disease.

Provided herein is a method of improving lung function in a subject comprising administering a therapeutically effective amount of a compound disclosed herein (e.g., a compound of Table 1 or Table 2, or a pharmaceutically acceptable salt or solvate thereof), or a pharmaceutical composition as provided herein, to the subject in need thereof. In some embodiments, the subject has been diagnosed as having lung fibrosis. In some embodiments, a compound disclosed herein (e.g., a compound of Table 1 or Table 2, or a pharmaceutically acceptable salt or solvate thereof), or a pharmaceutical composition as provided herein, is used to treat idiopathic pulmonary fibrosis in a subject. In some embodiments, a compound disclosed herein (e.g., a compound of Table 1 or Table 2, or a pharmaceutically acceptable salt or solvate thereof), or a pharmaceutical composition as provided herein, is used to treat usual interstitial pneumonia in a subject.

In some embodiments, a compound disclosed herein (e.g., a compound of Table 1 or Table 2, or a pharmaceutically acceptable salt or solvate thereof), or a pharmaceutical composition as provided herein is used to treat diffuse parenchymal interstitial lung diseases in subject such as iatrogenic drug induced, occupational/environmental induced fibrosis (Farmer lung), granulomatous diseases (sarcoidosis, hypersensitivity pneumonia), collagen vascular disease (scleroderma and others), alveolar proteinosis, langerhans cell granulonmatosis, lymphangioleiomyomatosis, inherited diseases (e.g., Hermansky-Pudlak Syndrome, Tuberous sclerosis, neurofibromatosis, metabolic storage disorders, and familial interstitial lung disease).

In some embodiments, a compound disclosed herein (e.g., a compound of Table 1 or Table 2, or a pharmaceutically acceptable salt or solvate thereof), or a pharmaceutical composition as provided herein is useful to treat post-transplant fibrosis associated with chronic rejection in a subject such as Bronchiolitis obliterans following a lung transplant.

In some embodiments, a compound disclosed herein (e.g., a compound of Table 1 or Table 2, or a pharmaceutically acceptable salt or solvate thereof), or a pharmaceutical composition as provided herein is useful to treat cutaneous fibrosis in a subject such as cutaneous scleroderma, Dupuytren disease, and keloids.

In some embodiments, a compound disclosed herein (e.g., a compound of Table 1 or Table 2, or a pharmaceutically acceptable salt or solvate thereof), or a pharmaceutical composition as provided herein is useful to treat hepatic fibrosis with or without cirrhosis in a subject. For example, toxic/drug induced (hemochromatosis), alcoholic liver disease, viral hepatitis (hepatitis B virus, hepatitis C virus, HCV), nonalcoholic liver disease (NAFLD, NASH), and metabolic and auto-immune disease.

In some embodiments, a compound disclosed herein (e.g., a compound of Table 1 or Table 2, or a pharmaceutically acceptable salt or solvate thereof), or a pharmaceutical composition as provided herein is useful to treat renal fibrosis in a subject (e.g., tubulointerstitium fibrosis and glomerular sclerosis).

Further examples of diseases, disorders, or conditions as provided herein include atherosclerosis, thrombosis, heart disease, vasculitis, formation of scar tissue, restenosis, phlebitis, COPD (chronic obstructive pulmonary disease), pulmonary hypertension, pulmonary fibrosis, pulmonary inflammation, bowel adhesions, bladder fibrosis and cystitis, fibrosis of the nasal passages, sinusitis, inflammation mediated by neutrophils, and fibrosis mediated by fibroblasts.

In some embodiments, a compound disclosed herein (e.g., a compound of Table 1 or Table 2, or a pharmaceutically acceptable salt or solvate thereof), or a pharmaceutical composition as provided herein, is administered to a subject with fibrosis of an organ or tissue or with a predisposition of developing fibrosis of an organ or tissue with one or more other agents that are used to treat fibrosis. In some embodiments, the one or more agents include corticosteroids, immunosuppressants, B-cell antagonists, and uteroglobin.

In some embodiments, a compound disclosed herein (e.g., a compound of Table 1 or Table 2, or a pharmaceutically acceptable salt or solvate thereof), or a pharmaceutical composition as provided herein, is used to treat a dermatological disorder in a subject. Such dermatological disorders include, but are not limited to, proliferative or inflammatory disorders of the skin such as, atopic dermatitis, bullous disorders, collagenoses, psoriasis, scleroderma, psoriatic lesions, dermatitis, contact dermatitis, eczema, urticaria, rosacea, wound healing, scarring, hypertrophic scarring, keloids, Kawasaki Disease, rosacea, Sjogren-Larsso Syndrome, or urticaria. In some embodiments, a compound disclosed herein (e.g., a compound of Table 1 or Table 2, or a pharmaceutically acceptable salt or solvate thereof) is used to treat systemic sclerosis.

In some embodiments, a compound disclosed herein (e.g., a compound of Table 1 or Table 2, or a pharmaceutically acceptable salt or solvate thereof) is useful to treat or prevent inflammation in a subject. For example, a compound disclosed herein (e.g., a compound of Table 1 or Table 2, or a pharmaceutically acceptable salt or solvate thereof) can be used in the treatment or prevention of inflammatory/immune disorders in a subject.

Examples of inflammatory/immune disorders include psoriasis, rheumatoid arthritis, vasculitis, inflammatory bowel disease, dermatitis, osteoarthritis, asthma, inflammatory muscle disease, allergic rhinitis, vaginitis, interstitial cystitis, scleroderma, eczema, allogeneic or xenogeneic transplantation (organ, bone marrow, stem cells and other cells and tissues) graft rejection, graft-versus-host disease, lupus erythematosus, inflammatory disease, type I diabetes, pulmonary fibrosis, dermatomyositis, Sjogren's syndrome, thyroiditis (e.g., Hashimoto's and autoimmune thyroiditis), myasthenia gravis, autoimmune hemolytic anemia, multiple sclerosis, cystic fibrosis, chronic relapsing hepatitis, primary biliary cirrhosis, allergic conjunctivitis and atopic dermatitis.

In some embodiments, a compound disclosed herein (e.g., a compound of Table 1 or Table 2, or a pharmaceutically acceptable salt or solvate thereof), or a pharmaceutical composition as provided herein, is used in the treatment of pain in a subject. In some embodiments, the pain is acute pain or chronic pain. In some embodiments, the pain is neuropathic pain.

In some embodiments, a compound disclosed herein (e.g., a compound of Table 1 or Table 2, or a pharmaceutically acceptable salt or solvate thereof), or a pharmaceutical composition as provided herein, is used in the treatment of fibromyalgia. Fibromyalgia is believed to stem from the formation of fibrous scar tissue in contractile (voluntary) muscles. Fibrosis binds the tissue and inhibits blood flow, resulting in pain.

In some embodiments, a compound disclosed herein (e.g., a compound of Table 1 or Table 2, or a pharmaceutically acceptable salt or solvate thereof), or a pharmaceutical composition as provided herein, is used in the treatment of cancer. In some embodiments, a compound disclosed herein (e.g., a compound of Table 1 or Table 2, or a pharmaceutically acceptable salt or solvate thereof), or a pharmaceutical composition as provided herein, is used in the treatment of malignant and benign proliferative disease. In some embodiments, a compound disclosed herein (e.g., a compound of Table 1 or Table 2, or a pharmaceutically acceptable salt or solvate thereof), or a pharmaceutical composition as provided herein, is used to prevent or reduce proliferation of tumor cells, invasion and metastasis of carcinomas, pleural mesothelioma (Yamada, Cancer Sci., 2008, 99(8), 1603-1610) or peritoneal mesothelioma, cancer pain, bone metastases (Boucharaba et al, J Clin. Invest., 2004, 114(12), 1714-1725; Boucharaba et al, Proc. Natl. Acad. Sci., 2006, 103(25) 9643-9648). Provided herein is a method of treating cancer in a subject, the method comprising administering to the subject a therapeutically effective amount a compound disclosed herein (e.g., a compound of Table 1 or Table 2, or a pharmaceutically acceptable salt or solvate thereof), or a pharmaceutical composition as provided herein. In some embodiments, the methods provided herein further include administration of a second therapeutic agent, wherein the second therapeutic agent is an anti-cancer agent.

The term “cancer,” as used herein refers to an abnormal growth of cells which tend to proliferate in an uncontrolled way and, in some cases, to metastasize (spread). The types of cancer include, but is not limited to, solid tumors (such as those of the bladder, bowel, brain, breast, endometrium, heart, kidney, lung, lymphatic tissue (lymphoma), ovary, pancreas or other endocrine organ (thyroid), prostate, skin (melanoma or basal cell cancer) or hematological tumors (such as the leukemias) at any stage of the disease with or without metastases.

Further non-limiting examples of cancers include, acute lymphoblastic leukemia, acute myeloid leukemia, adrenocortical carcinoma, anal cancer, appendix cancer, astrocytomas, atypical teratoid/rhabdoid tumor, basal cell carcinoma, bile duct cancer, bladder cancer, bone cancer (osteosarcoma and malignant fibrous histiocytoma), brain stem glioma, brain tumors, brain and spinal cord tumors, breast cancer, bronchial tumors, Burkitt lymphoma, cervical cancer, chronic lymphocytic leukemia, chronic myelogenous leukemia, colon cancer, colorectal cancer, craniopharyngioma, cutaneous T-Cell lymphoma, embryonal tumors, endometrial cancer, ependymoblastoma, ependymoma, esophageal cancer, Ewing sarcoma family of tumors, eye cancer, retinoblastoma, gallbladder cancer, gastric (stomach) cancer, gastrointestinal carcinoid tumor, gastrointestinal stromal tumor (GIST), gastrointestinal stromal cell tumor, germ cell tumor, glioma, hairy cell leukemia, head and neck cancer, hepatocellular (liver) cancer, Hodgkin lymphoma, hypopharyngeal cancer, intraocular melanoma, islet cell tumors (endocrine pancreas), Kaposi sarcoma, kidney cancer, Langerhans cell histiocytosis, laryngeal cancer, leukemia, Acute lymphoblastic leukemia, acute myeloid leukemia, chronic lymphocytic leukemia, chronic myelogenous leukemia, hairy cell leukemia, liver cancer, non-small cell lung cancer, small cell lung cancer, Burkitt lymphoma, cutaneous T-cell lymphoma, Hodgkin lymphoma, non-Hodgkin lymphoma, lymphoma. Waldenstrom macroglobulinemia, medulloblastoma, medulloepithelioma, melanoma, mesothelioma, mouth cancer, chronic myelogenous leukemia, myeloid leukemia, multiple myeloma, nasopharyngeal cancer, neuroblastoma, non-Hodgkin lymphoma, non-small cell lung cancer, oral cancer, oropharyngeal cancer, osteosarcoma, malignant fibrous histiocytoma of bone, ovarian cancer, ovarian epithelial cancer, ovarian germ cell tumor, ovarian low malignant potential tumor, pancreatic cancer, papillomatosis, parathyroid cancer, penile cancer, pharyngeal cancer, pineal parenchymal tumors of intermediate differentiation, pineoblastoma and supratentorial primitive neuroectodermal tumors, pituitary tumor, plasma cell neoplasm/multiple myeloma, pleuropulmonary blastoma, primary central nervous system lymphoma, prostate cancer, rectal cancer, renal cell (kidney) cancer, retinoblastoma, rhabdomyosarcoma, salivary gland cancer, sarcoma, Ewing sarcoma family of tumors, sarcoma, kaposi, Sezary syndrome, skin cancer, small cell Lung cancer, small intestine cancer, soft tissue sarcoma, squamous cell carcinoma, stomach (gastric) cancer, supratentorial primitive neuroectodermal tumors, T-cell lymphoma, testicular cancer, throat cancer, thymoma and thymic carcinoma, thyroid cancer, urethral cancer, uterine cancer, uterine sarcoma, vaginal cancer, vulvar cancer, Waldenstrom macroglobulinemia, and Wilms tumor.

In some embodiments, provided herein is a method of treating an allergic disorder in a subject, the method comprising administration of a therapeutically effective amount of a compound disclosed herein (e.g., a compound of Table 1 or Table 2, or a pharmaceutically acceptable salt or solvate thereof) as provided herein. In some embodiments, a compound disclosed herein (e.g., a compound of Table 1 or Table 2, or a pharmaceutically acceptable salt or solvate thereof), is useful for the treatment of respiratory diseases, disorders, or conditions in a subject. For example, a compound disclosed herein (e.g., a compound of Table 1 or Table 2, or a pharmaceutically acceptable salt or solvate thereof) can treat asthma (e.g., chronic asthma) in a subject.

The term “respiratory disease,” as used herein, refers to diseases affecting the organs that are involved in breathing, such as the nose, throat, larynx, eustachian tubes, trachea, bronchi, lungs, related muscles (e.g., diaphragm and intercostals), and nerves. Non-limiting examples of respiratory diseases include asthma, adult respiratory distress syndrome and allergic (extrinsic) asthma, non-allergic (intrinsic) asthma, acute severe asthma, chronic asthma, clinical asthma, nocturnal asthma, allergen-induced asthma, aspirin-sensitive asthma, exercise-induced asthma, isocapnic hyperventilation, child-onset asthma, adult-onset asthma, cough-variant asthma, occupational asthma, steroid-resistant asthma, seasonal asthma, seasonal allergic rhinitis, perennial allergic rhinitis, chronic obstructive pulmonary disease, including chronic bronchitis or emphysema, pulmonary hypertension, interstitial lung fibrosis and/or airway inflammation and cystic fibrosis, and hypoxia.

The term “asthma” as used herein refers to any disorder of the lungs characterized by variations in pulmonary gas flow associated with airway constriction of whatever cause (intrinsic, extrinsic, or both; allergic or non-allergic). The term asthma may be used with one or more adjectives to indicate cause.

Further provided herein are methods for treating or preventing chronic obstructive pulmonary disease in a subject comprising administering a therapeutically effective amount of a compound disclosed herein (e.g., a compound of Table 1 or Table 2, or a pharmaceutically acceptable salt or solvate thereof) as provided herein. Examples of chronic obstructive pulmonary disease include, but are not limited to, chronic bronchitis or emphysema, pulmonary hypertension, interstitial lung fibrosis and/or airway inflammation, and cystic fibrosis.

In some embodiments, a compound disclosed herein (e.g., a compound of Table 1 or Table 2, or a pharmaceutically acceptable salt or solvate thereof) is useful in the treatment or prevention of a nervous system disorder in a subject. The term “nervous system disorder,” as used herein, refers to conditions that alter the structure or function of the brain, spinal cord or peripheral nervous system, including but not limited to Alzheimer's Disease, cerebral edema, cerebral ischemia, stroke, multiple sclerosis, neuropathies, Parkinson's Disease, those found after blunt or surgical trauma (including post-surgical cognitive dysfunction and spinal cord or brain stem injury), as well as the neurological aspects of disorders such as degenerative disk disease and sciatica.

In some embodiments, provided herein is a method for treating or preventing a CNS disorder in a subject. Non-limiting examples of CNS disorders include multiple sclerosis, Parkinson's disease, Alzheimer's disease, stroke, cerebral ischemia, retinal ischemia, post-surgical cognitive dysfunction, migraine, peripheral neuropathy/neuropathic pain, spinal cord injury, cerebral edema and head injury.

Also provided herein are methods of treating or preventing cardiovascular disease in a subject. The term “cardiovascular disease,” as used herein refers to diseases affecting the heart or blood vessels or both, including but not limited to: arrhythmia (atrial or ventricular or both); atherosclerosis and its sequelae; angina; cardiac rhythm disturbances; myocardial ischemia; myocardial infarction; cardiac or vascular aneurysm; vasculitis, stroke; peripheral obstructive arteriopathy of a limb, an organ, or a tissue; reperfusion injury following ischemia of the brain, heart or other organ or tissue; endotoxic, surgical, or traumatic shock; hypertension, valvular heart disease, heart failure, abnormal blood pressure; shock; vasoconstriction (including that associated with migraines); vascular abnormality, inflammation, insufficiency limited to a single organ or tissue. For example, provided herein are methods for treating or preventing vasoconstriction, atherosclerosis and its sequelae myocardial ischemia, myocardial infarction, aortic aneurysm, vasculitis and stroke comprising administering a therapeutically effective amount of a compound disclosed herein (e.g., a compound of Table 1 or Table 2, or a pharmaceutically acceptable salt or solvate thereof).

In some embodiments, provided herein are methods for reducing cardiac reperfusion injury following myocardial ischemia and/or endotoxic shock comprising administering to a subject in need thereof a therapeutically effective amount of a compound disclosed herein (e.g., a compound of Table 1 or Table 2, or a pharmaceutically acceptable salt or solvate thereof).

Further provided herein are methods for reducing the constriction of blood vessels in a subject comprising administering a therapeutically effective amount of a compound disclosed herein (e.g., a compound of Table 1 or Table 2, or a pharmaceutically acceptable salt or solvate thereof). For example, methods for lowering or preventing an increase in blood pressure of a subject comprising administering a therapeutically effective amount of a compound disclosed herein (e.g., a compound of Table 1 or Table 2, or a pharmaceutically acceptable salt or solvate thereof) are provided herein.

The ability of test compounds to act as inhibitors of an LGA receptor can be demonstrated by assays known in the art. The activity of the compounds and compositions provided herein as LGA receptor inhibitors can be assayed in vitro, in vivo, or in a cell line.

For example, Chinese hamster ovary cells overexpressing human LPA1 can be plated overnight (15,000 cells/well) in microplates in DMEM/F12 medium. Following overnight culture, cells are loaded with calcium indicator dye for 30 minutes at 37° C. The cells are then equilibrated to room temperature for 30 minutes before the assay. Test compounds solubilized in DMSO are transferred to a multiwell non-binding surface plate and diluted with assay buffer (e.g., IX HBSS with calcium/magnesium, 20 mM HEPES, and 0.1% fatty acid free BSA) to a final concentration of 0.5% DMSO. Diluted compounds are added to the cells at final concentrations ranging from 0.08 nM to 5 mM and are then incubated for 20 min at room temperature at which time LPA is added at final concentrations of 10 nM to stimulate the cells. The compound IC₅₀ value is defined as the concentration of test compound which inhibited 50% of the calcium flux induced by LPA alone. IC₅₀ values can be determined by fitting data to a 4-parameter logistic equation.

In another example, a compound disclosed herein (e.g., a compound of Table 1 or Table 2, or a pharmaceutically acceptable salt or solvate thereof) as provided herein is dosed orally p.o. 2 hours to CD-1 female mice prior to an LPA challenge. The mice are then dosed via tail vein (IV) with 0.15 mL of LPA in 0.1% BSA/PBS (2 pg/pL). Exactly 2 minutes following the LPA challenge, the mice are euthanized by decapitation and the trunk blood is collected. These samples are collectively centrifuged and individual 75 pL samples are frozen at −20° C. until performance of a histamine assay. The plasma histamine analysis can be nm by standard EIA (Enzyme Immunoassay) methods. Plasma samples are thawed and diluted 1:30 in 0.1% BSA in PBS. An EIA protocol for histamine analysis as previously described can be used in this assay.

LPA has a role as a biological effector molecule, and has a diverse range of physiological actions that include effects on blood pressure, platelet activation, and smooth muscle contraction, and a variety of cellular effects, which include cell growth, cell rounding, neurite retraction, and actin stress fiber formation and cell migration. These effects are predominantly receptor mediated.

Activation of the LPA receptors (LPA₁, LPA₂, LPA₃, LPA₄, LPA₅, LPA₆) with LPA mediates a range of downstream signaling cascades. Non-limiting examples include, mitogen-activated protein kinase (MAPK) activation, adenylyl cyclase (AC) inhibition/activation, phospholipase C (PLC) activation/Ca2+ mobilization, arachidonic acid release, Akt/PKB activation, and the activation of small GTPases, Rho, ROCK, Rae, and Ras. Additional pathways that are affected by LPA receptor activation include, for example, cyclic adenosine monophosphate (cAMP), cell division cycle 42/GTP-binding protein (Cdc42), proto-oncogene serine/threonine-protein kinase Raf (c-RAF), proto-oncogene tyrosine-protein kinase Src (c-src), extracellular signal-regulated kinase (ERK), focal adhesion kinase (FAK), guanine nucleotide exchange factor (GEF), glycogen synthase kinase 3b (GSK3b), c-jun amino-terminal kinase (JNK). MEK, myosin light chain II (MLC II), nuclear factor kB (NF-kB), N-methyl-D-aspartate (NMDA) receptor activation, phosphatidylinositol 3-kinase (PBK), protein kinase A (PKA), protein kinase C (PKC), ms-related C3 botulinum toxin substrate 1 (RACI). Nearly all mammalian cells, tissues and organs co-express several LPA-receptor subtypes, which indicates that LPA receptors signal in a cooperative manner. LPA1, LPA2, and LPA3 share high amino acid sequence similarity.

LPA₁ (previously called VZG-1/EDG-2/mrecl.3) couples with three types of G proteins, G_{i/o}, G_{q}, and G_{12/13}. Through activation of these G proteins, LPA induces a range of cellular responses through LPA₁ including, for example, cell proliferation, serum-response element (SRE) activation, mitogen-activated protein kinase (MAPK) activation, adenylyl cyclase (AC) inhibition, phospholipase C (PLC) activation, Ca²⁺ mobilization, Akt activation, and Rho activation.

Expression of LPA₁ is observed in the testis, brain, heart, lung, small intestine, stomach, spleen, thymus, and skeletal muscle of in mice. Similarly, LPA₁ is expressed in human tissues such as the brain, heart, lung, placenta, colon, small intestine, prostate, testis, ovary, pancreas, spleen, kidney, skeletal muscle, and thymus.

LPA₂ (EDG-4) also couples with three types of G proteins, G_{i/o}, G_{q}, and G_{12/13}, to mediate LPA-induced cellular signaling. Expression of LPA₂ is observed in the testis, kidney, lung, thymus, spleen, and stomach of adult mice and in the human testis, pancreas, prostate, thymus, spleen, and peripheral blood leukocytes. Expression of LPA₂ is upregulated in various cancer cell lines, and several human LPA₂ transcriptional variants with mutations in the 3′-untranslated region have been observed.

LPA₃ can mediate pleiotropic LPA-induced signaling that includes PLC activation. Ca²⁺ mobilization, AC inhibition/activation, and MAPK activation. Overexpression of LPA₃ in neuroblastoma cells leads to neurite elongation. Expression of LPA₃ is observed in adult mouse testis, kidney, lung, small intestine, heart, thymus, and brain. In humans, it is found in the heart, pancreas, prostate, testis, lung, ovary, and brain (frontal cortex, hippocampus, and amygdala).

LPA₄ (p2y₉/GPR23) is of divergent sequence compared to LPA₁, LPA₂, and LPA₃ with closer similarity to the platelet-activating factor (PAF) receptor. LPA4 mediates LPA induced Ca²⁺ mobilization and cAMP accumulation, and functional coupling to the G protein Gs for AC activation, as well as coupling to other G proteins. The LPA₄ gene is expressed in the ovary, pancreas, thymus, kidney and skeletal muscle.

LPA₅(GPR92) is a member of the purinocluster of GPCRs and is structurally most closely related to LPA₄. LPA₅ is expressed in human heart, placenta, spleen, brain, lung and gut. LPAs also shows very high expression in the CD8+ lymphocyte compartment of the gastrointestinal tract.

LPA₆ (p2y5) is a member of the purinocluster of GPCRs and is structurally most closely related to LPA₄. LPA₆ is an LPA receptor coupled to the G12/13-Rho signaling pathways and is expressed in the inner root sheaths of human hair follicles.

Improvements in any of the foregoing response criteria are specifically provided by the methods of the present disclosure.

### Combination Therapies

In one embodiment, the compounds disclosed herein may be used in combination with one or more additional therapeutic agent that are being used and/or developed to treat a disease, disorder, or condition in which inhibition of one or more LPA receptors (i.e., an LPA-associated disease) is beneficial for the treatment of the underlying pathology and/or symptoms and/or progression of the disease, disorder, or condition . . . .

The compounds as provided herein, or pharmaceutically acceptable salts or solvates thereof, or pharmaceutical compositions of such compounds, are useful as inhibitors of one or more LPA receptors. As described further herein, a compound antagonizing to an LPA receptor can be useful for prevention and/or treatment of diseases such as various kinds of disease including, for example, fibrosis (e.g., renal fibrosis, pulmonary fibrosis, hepatic fibrosis, arterial fibrosis, systemic sclerosis), urinary system disease, carcinoma-associated disease, proliferative disease, inflammation/immune system disease, disease by secretory dysfunction, brain-related disease, and chronic disease.

In some embodiments, this disclosure provides methods for treating a subject (e.g., a human) having a disease, disorder, or condition in which inhibition of one or more LPA receptors (i.e., an LPA-associated disease) is beneficial for the treatment of the underlying pathology and/or symptoms and/or progression of the disease, disorder, or condition. In some embodiments, the methods provided herein can include or further include treating one or more conditions associated, co-morbid or sequela with any one or more of the conditions provided herein.

Provided herein is a method for treating a LPA-associated disease, the method comprising administering to a subject in need thereof an effective amount of a compound disclosed herein (e.g., a compound of Table 1 or Table 2, or a pharmaceutically acceptable salt or solvate thereof), or a pharmaceutical composition as disclosed herein.

In some embodiments, an LPA-associated disease includes, but is not limited to treating fibrosis of an organ (e.g., liver, kidney, lung, heart, and skin), liver disease (acute hepatitis, chronic hepatitis, liver fibrosis, liver cirrhosis, portal hypertension, regenerative failure, non-alcoholic steatohepatitis (NASH), liver hypofunction, hepatic blood flow disorder, and the like), cell proliferative disease (e.g., cancer, including solid tumors, solid tumor metastasis, vascular fibroma, myeloma, multiple myeloma, Kaposi's sarcoma, leukemia, and chronic lymphocytic leukemia (CLL), and invasive metastasis of cancer cells, inflammatory disease (e.g., psoriasis, nephropathy, and pneumonia), gastrointestinal tract disease (e.g., irritable bowel syndrome (TBS), inflammatory bowel disease (IBD), and abnormal pancreatic secretion), renal disease, urinary tract-associated disease (e.g., benign prostatic hyperplasia or symptoms associated with neuropathic bladder disease, spinal cord tumor, hernia of intervertebral disk, spinal canal stenosis, symptoms derived from diabetes, lower urinary tract disease (e.g., obstruction of lower urinary tract), inflammatory disease of the lower urinary tract, dysuna, and frequent urination), pancreas disease, abnormal angiogenesis-associated disease (e.g., arterial obstruction), scleroderma, brain-associated disease (e.g., cerebral infarction and cerebral hemorrhage), neuropathic pain, peripheral neuropathy, ocular disease (e.g., age-related macular degeneration (AMD), diabetic retinopathy, proliferative vitreoretinopathy (PVR), cicatricial pemphigoid, and glaucoma filtration surgery scarring).

In some embodiments, provided herein are methods of treating or preventing fibrosis, the method comprising administering to a subject in need thereof a therapeutically effective amount of a compound disclosed herein (e.g., a compound of Table 1 or Table 2, or a pharmaceutically acceptable salt or solvate thereof), or a pharmaceutical composition as disclosed herein. For example, the methods can include treating renal fibrosis, pulmonary fibrosis, hepatic fibrosis, arterial fibrosis or systemic sclerosis. In some embodiments, provided herein are methods of treating pulmonary fibrosis (e.g., Idiopathic Pulmonary Fibrosis (IPF)), the method comprising administering to a subject in need thereof a therapeutically effective amount of a compound disclosed herein (e.g., a compound of Table 1 or Table 2, or a pharmaceutically acceptable salt or solvate thereof), or a pharmaceutical composition as provided herein.

In some embodiments, a compound disclosed herein (e.g., a compound of Table 1 or Table 2, or a pharmaceutically acceptable salt or solvate thereof), or a pharmaceutical composition as provided herein, is used to treat or prevent fibrosis in a subject. For example, a compound disclosed herein (e.g., a compound of Table 1 or Table 2, or a pharmaceutically acceptable salt or solvate thereof), or a pharmaceutical composition as provided herein, can be used to treat fibrosis of an organ or tissue in a subject. In some embodiments, provided herein is a method for preventing a fibrosis condition in a subject, the method comprising administering to the subject at risk of developing one or more fibrosis conditions a therapeutically effective amount of a compound disclosed herein (e.g., a compound of Table 1 or Table 2, or a pharmaceutically acceptable salt or solvate thereof), or a pharmaceutical composition as provided herein. For example, the subject may have been exposed to one or more environmental conditions that are known to increase the risk of fibrosis of an organ or tissue. In some embodiments, the subject has been exposed to one or more environmental conditions that are known to increase the risk of lung, liver or kidney fibrosis. In some embodiments, the subject has a genetic predisposition of developing fibrosis of an organ or tissue. In some embodiments, a compound disclosed herein (e.g., a compound of Table 1 or Table 2, or a pharmaceutically acceptable salt or solvate thereof), or a pharmaceutical composition as provided herein, is administered to a subject to prevent or minimize scarring following injury. For example, the injury can include surgery.

Exemplary diseases, disorders, or conditions that involve fibrosis include, but are not limited to: lung diseases associated with fibrosis, for example, idiopathic pulmonary fibrosis, iatrogenic drug induced, occupational/environmental induced fibrosis (Farmer lung), granulomatous diseases (sarcoidosis, hypersensitivity pneumonia), collagen vascular disease (scleroderma and others), alveolar proteinosis, langerhans cell granulonmatosis, lymphangioleiomyomatosis, inherited diseases (e.g., Hermansky-Pudlak Syndrome, Tuberous sclerosis, neurofibromatosis, metabolic storage disorders, and familial interstitial lung disease), pulmonary fibrosis secondary to systemic inflammatory disease such as rheumatoid arthritis, scleroderma, lupus, cryptogenic fibrosing alveolitis, radiation induced fibrosis, chronic obstructive pulmonary disease (COPD), scleroderma, bleomycin induced pulmonary fibrosis, chronic asthma, silicosis, asbestos induced pulmonary or pleural fibrosis, acute lung injury, acute respiratory distress syndrome (ARDS), and acute respiratory distress (including bacterial pneumonia induced, trauma induced, viral pneumonia induced, ventilator induced, non-pulmonary sepsis induced, and aspiration induced). Chronic nephropathies associated with injury/fibrosis, kidney fibrosis (renal fibrosis), glomerulonephritis secondary to systemic inflammatory diseases such as lupus and scleroderma, tubulointerstitium fibrosis, glomerular nephritis, glomerular sclerosis, focal segmental, diabetes, glomerular nephritis, focal segmental glomerular sclerosis, IgA nephropathy, hypertension, allograft and Alport Syndrome; dermatological disorders, gut fibrosis, for example, scleroderma, and radiation induced gut fibrosis; liver fibrosis, for example, cirrhosis, alcohol induced liver fibrosis, nonalcoholic steatohepatitis (NASH), non-alcoholic fatty liver disease (NAFLD), toxic/drug induced liver fibrosis (e.g., hemochromatosis), biliary duct injury, primary biliary cirrhosis, infection or viral induced liver fibrosis (e.g., chronic HCV infection), inflammatory/immune disorders, and autoimmune hepatitis; head and neck fibrosis, for example, corneal scarring, e.g., LASIK (laser-assisted in situ keratomileusis), corneal transplant, and trabeculectomy; hypertrophic scarring, Duputren disease, cutaneous fibrosis, cutaneous scleroderma, keloids, e.g., burn induced or surgical; and other fibrotic diseases, e.g., sarcoidosis, scleroderma, spinal cord injury/fibrosis, myelofibrosis, vascular restenosis, atherosclerosis, arteriosclerosis, Wegener's granulomatosis, chronic lymphocytic leukemia, tumor metastasis, transplant organ rejection (e.g., Bronchiolitis obliterans), endometriosis, neonatal respiratory distress syndrome, and neuropathic pain, fibromyalgia, mixed connective tissue disease, and Peyronie's disease.

Provided herein is a method of improving lung function in a subject comprising administering a therapeutically effective amount of a compound disclosed herein (e.g., a compound of Table 1 or Table 2, or a pharmaceutically acceptable salt or solvate thereof), or a pharmaceutical composition as provided herein, to the subject in need thereof. In some embodiments, the subject has been diagnosed as having lung fibrosis. In some embodiments, a compound disclosed herein (e.g., a compound of Table 1 or Table 2, or a pharmaceutically acceptable salt or solvate thereof), or a pharmaceutical composition as provided herein, is used to treat idiopathic pulmonary fibrosis in a subject. In some embodiments, a compound disclosed herein (e.g., a compound of Table 1 or Table 2, or a pharmaceutically acceptable salt or solvate thereof), or a pharmaceutical composition as provided herein, is used to treat usual interstitial pneumonia in a subject.

In some embodiments, a compound disclosed herein (e.g., a compound of Table 1 or Table 2, or a pharmaceutically acceptable salt or solvate thereof), or a pharmaceutical composition as provided herein is used to treat diffuse parenchymal interstitial lung diseases in subject such as iatrogenic drug induced, occupational/environmental induced fibrosis (Farmer lung), granulomatous diseases (sarcoidosis, hypersensitivity pneumonia), collagen vascular disease (scleroderma and others), alveolar proteinosis, langerhans cell granulonmatosis, lymphangioleiomyomatosis, inherited diseases (e.g., Hermansky-Pudlak Syndrome, Tuberous sclerosis, neurofibromatosis, metabolic storage disorders, and familial interstitial lung disease).

In some embodiments, a compound disclosed herein (e.g., a compound of Table 1 or Table 2, or a pharmaceutically acceptable salt or solvate thereof), or a pharmaceutical composition as provided herein is useful to treat post-transplant fibrosis associated with chronic rejection in a subject such as Bronchiolitis obliterans following a lung transplant.

In some embodiments, a compound disclosed herein (e.g., a compound of Table 1 or Table 2, or a pharmaceutically acceptable salt or solvate thereof), or a pharmaceutical composition as provided herein is useful to treat cutaneous fibrosis in a subject such as cutaneous scleroderma, Dupuytren disease, and keloids.

In some embodiments, a compound disclosed herein (e.g., a compound of Table 1 or Table 2, or a pharmaceutically acceptable salt or solvate thereof), or a pharmaceutical composition as provided herein is useful to treat hepatic fibrosis with or without cirrhosis in a subject. For example, toxic/drug induced (hemochromatosis), alcoholic liver disease, viral hepatitis (hepatitis B virus, hepatitis C virus, HCV), nonalcoholic liver disease (NAFLD, NASH), and metabolic and auto-immune disease.

In some embodiments, a compound disclosed herein (e.g., a compound of Table 1 or Table 2, or a pharmaceutically acceptable salt or solvate thereof), or a pharmaceutical composition as provided herein is useful to treat renal fibrosis in a subject (e.g., tubulointerstitium fibrosis and glomerular sclerosis).

Further examples of diseases, disorders, or conditions as provided herein include atherosclerosis, thrombosis, heart disease, vasculitis, formation of scar tissue, restenosis, phlebitis, COPD (chronic obstructive pulmonary disease), pulmonary hypertension, pulmonary fibrosis, pulmonary inflammation, bowel adhesions, bladder fibrosis and cystitis, fibrosis of the nasal passages, sinusitis, inflammation mediated by neutrophils, and fibrosis mediated by fibroblasts.

In some embodiments, a compound disclosed herein (e.g., a compound of Table 1 or Table 2, or a pharmaceutically acceptable salt or solvate thereof), or a pharmaceutical composition as provided herein is useful to treat one or more symptoms of COVID-19.

In some embodiments, a compound disclosed herein (e.g., a compound of Table 1 or Table 2, or a pharmaceutically acceptable salt or solvate thereof), or a pharmaceutical composition as provided herein is useful to treat chronic obstructive pulmonary disease (COPD).

In some embodiments, a compound disclosed herein (e.g., a compound of Table 1 or Table 2, or a pharmaceutically acceptable salt or solvate thereof), or a pharmaceutical composition as provided herein is useful to treat neuroinflammation.

In some embodiments, a compound disclosed herein (e.g., a compound of Table 1 or Table 2, or a pharmaceutically acceptable salt or solvate thereof), or a pharmaceutical composition as provided herein is useful to treat multiple sclerosis.

In some embodiments, a compound disclosed herein (e.g., a compound of Table 1 or Table 2, or a pharmaceutically acceptable salt or solvate thereof), or a pharmaceutical composition as provided herein, is administered to a subject with fibrosis of an organ or tissue or with a predisposition of developing fibrosis of an organ or tissue with one or more other agents that are used to treat fibrosis. In some embodiments, the one or more agents include corticosteroids, immunosuppressants, B-cell antagonists, and uteroglobin.

In some embodiments, a compound disclosed herein (e.g., a compound of Table 1 or Table 2, or a pharmaceutically acceptable salt or solvate thereof), or a pharmaceutical composition as provided herein, is used to treat a dermatological disorder in a subject. Such dermatological disorders include, but are not limited to, proliferative or inflammatory disorders of the skin such as, atopic dermatitis, bullous disorders, collagenoses, psoriasis, scleroderma, psoriatic lesions, dermatitis, contact dermatitis, eczema, urticaria, rosacca, wound healing, scarring, hypertrophic scarring, keloids, Kawasaki Disease, rosacca, Sjogren-Larsso Syndrome, or urticaria. In some embodiments, a compound disclosed herein (e.g., a compound of Table 1 or Table 2, or a pharmaceutically acceptable salt or solvate thereof) is used to treat systemic sclerosis.

In some embodiments, a compound disclosed herein (e.g., a compound of Table 1 or Table 2, or a pharmaceutically acceptable salt or solvate thereof) is useful to treat or prevent inflammation in a subject. For example, a compound disclosed herein (e.g., a compound of Table 1 or Table 2, or a pharmaceutically acceptable salt or solvate thereof) can be used in the treatment or prevention of inflammatory/immune disorders in a subject.

Examples of inflammatory/immune disorders include psoriasis, rheumatoid arthritis, vasculitis, inflammatory bowel disease, dermatitis, osteoarthritis, asthma, inflammatory muscle disease, allergic rhinitis, vaginitis, interstitial cystitis, scleroderma, eczema, allogeneic or xenogeneic transplantation (organ, bone marrow, stem cells and other cells and tissues) graft rejection, graft-versus-host disease, lupus erythematosus, inflammatory disease, type I diabetes, pulmonary fibrosis, dermatomyositis, Sjogren's syndrome, thyroiditis (e.g., Hashimoto's and autoimmune thyroiditis), myasthenia gravis, autoimmune hemolytic anemia, multiple sclerosis, cystic fibrosis, chronic relapsing hepatitis, primary biliary cirrhosis, allergic conjunctivitis and atopic dermatitis.

In some embodiments, a compound disclosed herein (e.g., a compound of Table 1 or Table 2, or a pharmaceutically acceptable salt or solvate thereof), or a pharmaceutical composition as provided herein, is used in the treatment of pain in a subject. In some embodiments, the pain is acute pain or chronic pain. In some embodiments, the pain is neuropathic pain.

In some embodiments, a compound disclosed herein (e.g., a compound of Table 1 or Table 2, or a pharmaceutically acceptable salt or solvate thereof), or a pharmaceutical composition as provided herein, is used in the treatment of fibromyalgia. Fibromyalgia is believed to stem from the formation of fibrous scar tissue in contractile (voluntary) muscles. Fibrosis binds the tissue and inhibits blood flow, resulting in pain.

In some embodiments, a compound disclosed herein (e.g., a compound of Table 1 or Table 2, or a pharmaceutically acceptable salt or solvate thereof), or a pharmaceutical composition as provided herein, is used in the treatment of cancer. In some embodiments, a compound disclosed herein (e.g., a compound of Table 1 or Table 2, or a pharmaceutically acceptable salt or solvate thereof), or a pharmaceutical composition as provided herein, is used in the treatment of malignant and benign proliferative disease. In some embodiments, a compound disclosed herein (e.g., a compound of Table 1 or Table 2, or a pharmaceutically acceptable salt or solvate thereof), or a pharmaceutical composition as provided herein, is used to prevent or reduce proliferation of tumor cells, invasion and metastasis of carcinomas, pleural mesothelioma (Yamada, Cancer Sci., 2008, 99(8), 1603-1610) or peritoneal mesothelioma, cancer pain, bone metastases (Boucharaba et al, J Clin. Invest., 2004, 114(12), 1714-1725; Boucharaba et al, Proc. Natl. Acad. Sci., 2006, 103(25) 9643-9648). Provided herein is a method of treating cancer in a subject, the method comprising administering to the subject a therapeutically effective amount a compound disclosed herein (e.g., a compound of Table 1 or Table 2, or a pharmaceutically acceptable salt or solvate thereof), or a pharmaceutical composition as provided herein. In some embodiments, the methods provided herein further include administration of a second therapeutic agent, wherein the second therapeutic agent is an anti-cancer agent.

The term “cancer,” as used herein refers to an abnormal growth of cells which tend to proliferate in an uncontrolled way and, in some cases, to metastasize (spread). The types of cancer include, but is not limited to, solid tumors (such as those of the bladder, bowel, brain, breast, endometrium, heart, kidney, lung, lymphatic tissue (lymphoma), ovary, pancreas or other endocrine organ (thyroid), prostate, skin (melanoma or basal cell cancer) or hematological tumors (such as the leukemias) at any stage of the disease with or without metastases.

Further non-limiting examples of cancers include, acute lymphoblastic leukemia, acute myeloid leukemia, adrenocortical carcinoma, anal cancer, appendix cancer, astrocytomas, atypical teratoid/rhabdoid tumor, basal cell carcinoma, bile duct cancer, bladder cancer, bone cancer (osteosarcoma and malignant fibrous histiocytoma), brain stem glioma, brain tumors, brain and spinal cord tumors, breast cancer, bronchial tumors, Burkitt lymphoma, cervical cancer, chronic lymphocytic leukemia, chronic myelogenous leukemia, colon cancer, colorectal cancer, craniopharyngioma, cutaneous T-Cell lymphoma, embryonal tumors, endometrial cancer, ependymoblastoma, ependymoma, esophageal cancer, Ewing sarcoma family of tumors, eye cancer, retinoblastoma, gallbladder cancer, gastric (stomach) cancer, gastrointestinal carcinoid tumor, gastrointestinal stromal tumor (GIST), gastrointestinal stromal cell tumor, germ cell tumor, glioma, hairy cell leukemia, head and neck cancer, hepatocellular (liver) cancer, Hodgkin lymphoma, hypopharyngeal cancer, intraocular melanoma, islet cell tumors (endocrine pancreas), Kaposi sarcoma, kidney cancer, Langerhans cell histiocytosis, laryngeal cancer, leukemia, Acute lymphoblastic leukemia, acute myeloid leukemia, chronic lymphocytic leukemia, chronic myelogenous leukemia, hairy cell leukemia, liver cancer, non-small cell lung cancer, small cell lung cancer, Burkitt lymphoma, cutaneous T-cell lymphoma, Hodgkin lymphoma, non-Hodgkin lymphoma, lymphoma. Waldenstrom macroglobulinemia, medulloblastoma, medulloepithelioma, melanoma, mesothelioma, mouth cancer, chronic myelogenous leukemia, myeloid leukemia, multiple myeloma, nasopharyngeal cancer, neuroblastoma, non-Hodgkin lymphoma, non-small cell lung cancer, oral cancer, oropharyngeal cancer, osteosarcoma, malignant fibrous histiocytoma of bone, ovarian cancer, ovarian epithelial cancer, ovarian germ cell tumor, ovarian low malignant potential tumor, pancreatic cancer, papillomatosis, parathyroid cancer, penile cancer, pharyngeal cancer, pineal parenchymal tumors of intermediate differentiation, pineoblastoma and supratentorial primitive neuroectodermal tumors, pituitary tumor, plasma cell neoplasm/multiple myeloma, pleuropulmonary blastoma, primary central nervous system lymphoma, prostate cancer, rectal cancer, renal cell (kidney) cancer, retinoblastoma, rhabdomyosarcoma, salivary gland cancer, sarcoma, Ewing sarcoma family of tumors, sarcoma, kaposi, Sezary syndrome, skin cancer, small cell Lung cancer, small intestine cancer, soft tissue sarcoma, squamous cell carcinoma, stomach (gastric) cancer, supratentorial primitive neuroectodermal tumors, T-cell lymphoma, testicular cancer, throat cancer, thymoma and thymic carcinoma, thyroid cancer, urethral cancer, uterine cancer, uterine sarcoma, vaginal cancer, vulvar cancer, Waldenstrom macroglobulinemia, and Wilms tumor.

In some embodiments, provided herein is a method of treating an allergic disorder in a subject, the method comprising administration of a therapeutically effective amount of a compound disclosed herein (e.g., a compound of Table 1 or Table 2, or a pharmaceutically acceptable salt or solvate thereof) as provided herein. In some embodiments, a compound disclosed herein (e.g., a compound of Table 1 or Table 2, or a pharmaceutically acceptable salt or solvate thereof), is useful for the treatment of respiratory diseases, disorders or conditions in a subject. For example, a compound disclosed herein (e.g., a compound of Table 1 or Table 2, or a pharmaceutically acceptable salt or solvate thereof) can treat asthma (e.g., chronic asthma) in a subject.

The term “respiratory disease,” as used herein, refers to diseases affecting the organs that are involved in breathing, such as the nose, throat, larynx, eustachian tubes, trachea, bronchi, lungs, related muscles (e.g., diaphragm and intercostals), and nerves. Non-limiting examples of respiratory diseases include asthma, adult respiratory distress syndrome and allergic (extrinsic) asthma, non-allergic (intrinsic) asthma, acute severe asthma, chronic asthma, clinical asthma, nocturnal asthma, allergen-induced asthma, aspirin-sensitive asthma, exercise-induced asthma, isocapnic hyperventilation, child-onset asthma, adult-onset asthma, cough-variant asthma, occupational asthma, steroid-resistant asthma, seasonal asthma, seasonal allergic rhinitis, perennial allergic rhinitis, chronic obstructive pulmonary disease, including chronic bronchitis or emphysema, pulmonary hypertension, interstitial lung fibrosis and/or airway inflammation and cystic fibrosis, and hypoxia.

The term “asthma” as used herein refers to any disorder of the lungs characterized by variations in pulmonary gas flow associated with airway constriction of whatever cause (intrinsic, extrinsic, or both; allergic or non-allergic). The term asthma may be used with one or more adjectives to indicate cause.

Further provided herein are methods for treating or preventing chronic obstructive pulmonary disease in a subject comprising administering a therapeutically effective amount of a compound disclosed herein (e.g., a compound of Table 1 or Table 2, or a pharmaceutically acceptable salt or solvate thereof) as provided herein. Examples of chronic obstructive pulmonary disease include, but are not limited to, chronic bronchitis or emphysema, pulmonary hypertension, interstitial lung fibrosis and/or airway inflammation, and cystic fibrosis.

In some embodiments, a compound disclosed herein (e.g., a compound of Table 1 or Table 2, or a pharmaceutically acceptable salt or solvate thereof) is useful in the treatment or prevention of a nervous system disorder in a subject. The term “nervous system disorder,” as used herein, refers to conditions that alter the structure or function of the brain, spinal cord or peripheral nervous system, including but not limited to Alzheimer's Disease, cerebral edema, cerebral ischemia, stroke, multiple sclerosis, neuropathies, Parkinson's Disease, those found after blunt or surgical trauma (including post-surgical cognitive dysfunction and spinal cord or brain stem injury), as well as the neurological aspects of disorders such as degenerative disk disease and sciatica.

In some embodiments, provided herein is a method for treating or preventing a CNS disorder in a subject. Non-limiting examples of CNS disorders include multiple sclerosis, Parkinson's disease, Alzheimer's disease, stroke, cerebral ischemia, retinal ischemia, post-surgical cognitive dysfunction, migraine, peripheral neuropathy/neuropathic pain, spinal cord injury, cerebral edema and head injury.

Also provided herein are methods of treating or preventing cardiovascular disease in a subject. The term “cardiovascular disease,” as used herein refers to diseases affecting the heart or blood vessels or both, including but not limited to: arrhythmia (atrial or ventricular or both); atherosclerosis and its sequelae; angina; cardiac rhythm disturbances; myocardial ischemia myocardial infarction; cardiac or vascular aneurysm; vasculitis, stroke; peripheral obstructive arteriopathy of a limb, an organ, or a tissue; reperfusion injury following ischemia of the brain, heart or other organ or tissue; endotoxic, surgical, or traumatic shock; hypertension, valvular heart disease, heart failure, abnormal blood pressure; shock; vasoconstriction (including that associated with migraines); vascular abnormality, inflammation, insufficiency limited to a single organ or tissue. For example, provided herein are methods for treating or preventing vasoconstriction, atherosclerosis and its sequelae myocardial ischemia, myocardial infarction, aortic aneurysm, vasculitis and stroke comprising administering a therapeutically effective amount of a compound disclosed herein (e.g., a compound of Table 1 or Table 2, or a pharmaceutically acceptable salt or solvate thereof).

In some embodiments, provided herein are methods for reducing cardiac reperfusion injury following myocardial ischemia and/or endotoxic shock comprising administering to a subject in need thereof a therapeutically effective amount of a compound disclosed herein (e.g., a compound of Table 1 or Table 2, or a pharmaceutically acceptable salt or solvate thereof).

Further provided herein are methods for reducing the constriction of blood vessels in a subject comprising administering a therapeutically effective amount of a compound disclosed herein (e.g., a compound of Table 1 or Table 2, or a pharmaceutically acceptable salt or solvate thereof). For example, methods for lowering or preventing an increase in blood pressure of a subject comprising administering a therapeutically effective amount of a compound disclosed herein (e.g., a compound of Table 1 or Table 2, or a pharmaceutically acceptable salt or solvate thereof) are provided herein.

### Pharmaceutical Compositions and Modes of Administration

Compounds provided herein are usually administered in the form of pharmaceutical compositions.

When employed as pharmaceuticals, a compound disclosed herein (e.g., a compound of Table 1 or Table 2, or a pharmaceutically acceptable salt or solvate thereof), including pharmaceutically acceptable salts or solvates thereof, can be administered in the form of a pharmaceutical compositions. These compositions can be prepared in a manner well known in the pharmaceutical art, and can be administered by a variety of routes, depending upon whether local or systemic treatment is desired and upon the area to be treated. Administration can be topical (including transdermal, epidermal, ophthalmic and to mucous membranes including intranasal, vaginal and rectal delivery), pulmonary (e.g., by inhalation or insufflation of powders or aerosols, including by nebulizer; intratracheal or intranasal), oral or parenteral. Oral administration can include a dosage form formulated for once-daily or twice-daily (BID) administration. Parenteral administration includes intravenous, intraarterial, subcutaneous, intraperitoneal intramuscular or injection or infusion; or intracranial, e.g., intrathecal or intraventricular, administration. Parenteral administration can be in the form of a single bolus dose, or can be, for example, by a continuous perfusion pump. Pharmaceutical compositions and formulations for topical administration can include transdermal patches, ointments, lotions, creams, gels, drops, suppositories, sprays, liquids and powders. Conventional pharmaceutical carriers, aqueous, powder or oily bases, thickeners and the like may be necessary or desirable.

Also provided herein are pharmaceutical compositions which contain, as the active ingredient, a compound disclosed herein, or a pharmaceutically acceptable salt or solvate thereof, (e.g., a compound of Table 1 or Table 2, or a pharmaceutically acceptable salt or solvate thereof), in combination with one or more pharmaceutically acceptable excipients (carriers). For example, a pharmaceutical composition prepared using a compound disclosed herein, or a pharmaceutically acceptable salt or solvate thereof, (e.g., a compound of Table 1 or Table 2, or a pharmaceutically acceptable salt or solvate thereof). In some embodiments, the composition is suitable for topical administration. In making the compositions provided herein, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders. In some embodiments, the composition is formulated for oral administration. In some embodiments, the composition is a solid oral formulation. In some embodiments, the composition is formulated as a tablet or capsule.

Further provided herein are pharmaceutical compositions containing a compound disclosed herein, or a pharmaceutically acceptable salt or solvate thereof, (e.g., a compound of Table 1 or Table 2, or a pharmaceutically acceptable salt or solvate thereof) with a pharmaceutically acceptable excipient. Pharmaceutical compositions containing a compound disclosed herein, or a pharmaceutically acceptable salt or solvate thereof, (e.g., a compound of Table 1 or Table 2, or a pharmaceutically acceptable salt or solvate thereof) as the active ingredient can be prepared by intimately mixing a compound disclosed herein, or a pharmaceutically acceptable salt or solvate thereof, (e.g., a compound of Table 1 or Table 2, or a pharmaceutically acceptable salt or solvate thereof) with a pharmaceutical carrier according to conventional pharmaceutical compounding techniques. The carrier can take a wide variety of forms depending upon the desired route of administration (e.g., oral, parenteral). In some embodiments, the composition is a solid oral composition.

Suitable pharmaceutically acceptable carriers are well known in the art. Descriptions of some of these pharmaceutically acceptable carriers can be found in The Handbook of Pharmaceutical Excipients, published by the American Pharmaceutical Association and the Pharmaceutical Society of Great Britain.

Methods of formulating pharmaceutical compositions have been described in numerous publications such as Pharmaceutical Dosage Forms: Tablets, Second Edition, Revised and Expanded, Volumes 1-3, edited by Lieberman et al; Pharmaceutical Dosage Forms: Parenteral Medications, Volumes 1-2, edited by Avis et al; and Pharmaceutical Dosage Forms: Disperse Systems. Volumes 1-2, edited by Lieberman et al; published by Marcel Dekker, Inc.

Pharmaceutically acceptable excipients include, but are not limited to, ion exchangers, alumina, aluminum stearate, lecithin, self-emulsifying drug delivery systems (SEDDS) such as d-α-tocopherol polyethylene glycol 1000 succinate, surfactants used in pharmaceutical dosage forms such as Tweens, poloxamers or other similar polymeric delivery matrices, serum proteins, such as human serum albumin, buffer substances such as phosphates, tris, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium-chloride, zinc salts, colloidal silica, magnesium trisilicate, polyvinyl pyrrolidone, cellulose-based substances, polyethylene glycol, sodium carboxymethyl cellulose, polyacrylates, waxes, polyethylene-polyoxypropylene-block polymers, and wool fat. Cyclodextrins such as α-, β, and γ-cyclodextrin, or chemically modified derivatives such as hydroxyalkylcyclodextrins, including 2- and 3-hydroxypropyl-β-cyclodextrins, or other solubilized derivatives can also be used to enhance delivery of compounds as provided herein. Dosage forms or compositions containing a chemical entity as provided herein in the range of 0.005% to 100% with the balance made up from non-toxic excipient may be prepared. The contemplated compositions may contain 0.001%-100% of a chemical entity provided herein, in one embodiment 0.1-95%, in another embodiment 75-85%, in a further embodiment 20-80%. Actual methods of preparing such dosage forms are known, or will be apparent, to those skilled in this art; for example, see Remington: The Science and Practice of Pharmacy, 22nd Edition (Pharmaceutical Press, London, U K. 2012).

In some embodiments, a compound disclosed herein, or a pharmaceutically acceptable salt or solvate thereof, (e.g., a compound of Table 1 or Table 2, or a pharmaceutically acceptable salt or solvate thereof), or pharmaceutical compositions as provided herein can be administered to a subject in need thereof by any accepted route of administration. Acceptable routes of administration include, but are not limited to, buccal, cutaneous, endocervical, endosinusial, endotracheal, enteral, epidural, interstitial, intra-abdominal, intra-arterial, intrabronchial, intrabursal, intracerebral, intracisternal, intracoronary, intradermal, intraductal, intraduodenal, intradural, intraepidermal, intraesophageal, intragastric, intragingival, intraileal, intralymphatic, intramedullary, intrameningeal, intramuscular, intraovarian, intraperitoneal, intraprostatic, intrapulmonary, intrasinal, intraspinal, intrasynovial, intratesticular, intrathecal, intratubular, intratumoral, intrauterine, intravascular, intravenous, nasal (e.g., intranasal), nasogastric, oral, parenteral, percutaneous, peridural, rectal, respiratory (inhalation), subcutaneous, sublingual, submucosal, topical, transdermal, transmucosal, transtracheal, ureteral, urethral and vaginal. In some embodiments, a preferred route of administration is parenteral (e.g., intratumoral).

In some embodiments, a compound disclosed herein (e.g., a compound of Table 1 or Table 2, or a pharmaceutically acceptable salt or solvate thereof) as provided herein or pharmaceutical compositions thereof can be formulated for parenteral administration, e.g., formulated for injection via the intraarterial, intrasternal, intracranial, intravenous, intramuscular, sub-cutaneous, or intraperitoneal routes. For example, such compositions can be prepared as injectables, either as liquid solutions or suspensions; solid forms suitable for use to prepare solutions or suspensions upon the addition of a liquid prior to injection can also be prepared, and the preparations can also be emulsified. The preparation of such formulations will be known to those of skill in the art in light of the present disclosure. In some embodiments, devices are used for parenteral administration. For example, such devices may include needle injectors, microneedle injectors, needle-free injectors, and infusion techniques.

In some embodiments, the pharmaceutical forms suitable for injection include sterile aqueous solutions or dispersions; formulations including sesame oil, peanut oil, or aqueous propylene glycol; and sterile powders for the extemporaneous preparation of sterile injectable solutions or dispersions. In some embodiments, the form must be sterile and must be fluid to the extent that it may be easily injected. In some embodiments, the form should be stable under the conditions of manufacture and storage and must be preserved against the contaminating action of microorganisms, such as bacteria and fungi.

In some embodiments, the carrier also can be a solvent or dispersion medium containing, for example, water, ethanol, polyol (for example, glycerol, propylene glycol, and liquid polyethylene glycol, and the like), suitable mixtures thereof, and vegetable oils. In some embodiments, the proper fluidity can be maintained, for example, by the use of a coating, such as lecithin, by the maintenance of the required particle size in the case of dispersion, and by the use of surfactants. In some embodiments, the prevention of the action of microorganisms can be brought about by various antibacterial and antifungal agents, for example, parabens, chlorobutanol, phenol, sorbic acid, thimerosal, and the like. In some embodiments, isotonic agents, for example, sugars or sodium chloride are included. In some embodiments, prolonged absorption of the injectable compositions can be brought about by the use in the compositions of agents delaying absorption, for example, aluminum monostearate and gelatin.

In some embodiments, sterile injectable solutions are prepared by incorporating a compound disclosed herein (e.g., a compound of Table 1 or Table 2, or a pharmaceutically acceptable salt or solvate thereof) in the required amount in the appropriate solvent with various of the other ingredients enumerated above, as required, followed by filtered sterilization. In some embodiments, dispersions are prepared by incorporating the various sterilized active ingredients into a sterile vehicle which contains the basic dispersion medium and the required other ingredients from those enumerated above. In some embodiments, sterile powders are used for the preparation of sterile injectable solutions. In some embodiments, the methods of preparation are vacuum-drying and freeze-drying techniques, which yield a powder of the active ingredient, plus any additional desired ingredient from a previously sterile-filtered solution thereof.

In some embodiments, pharmacologically acceptable excipients usable in a rectal composition as a gel, cream, enema, or rectal suppository, include, without limitation, any one or more of cocoa butter glycerides, synthetic polymers such as polyvinylpyrrolidone, PEG (like PEG ointments), glycerine, glycerinated gelatin, hydrogenated vegetable oils, poloxamers, mixtures of polyethylene glycols of various molecular weights and fatty acid esters of polyethylene glycol. Vaseline, anhydrous lanolin, shark liver oil, sodium saccharinate, menthol, sweet almond oil, sorbitol, sodium benzoate, anoxid SBN, vanilla essential oil, aerosol, parabens in phenoxyethanol, sodium methyl p-oxybenzoate, sodium propyl p-oxybenzoate, diethylamine, carbomers, carbopol, methyloxybenzoate, macrogol cetostearyl ether, cocoyl caprylocaprate, isopropyl alcohol, propylene glycol, liquid paraffin, xanthan gum, carboxy-metabisulfite, sodium edetate, sodium benzoate, potassium metabisulfite, grapefruit seed extract, methyl sulfonyl methane (MSM), lactic acid, glycine, vitamins, such as vitamin A and E and potassium acetate.

In some embodiments, suppositories can be prepared by mixing a compound disclosed herein (e.g., a compound of Table 1 or Table 2, or a pharmaceutically acceptable salt or solvate thereof) or pharmaceutical compositions as provided herein with suitable non-irritating excipients or carriers such as cocoa butter, polyethylene glycol or a suppository wax which are solid at ambient temperature but liquid at body temperature and therefore melt in the rectum and release the active compound. In some embodiments, compositions for rectal administration are in the form of an enema.

In some embodiments, a compound disclosed herein (e.g., a compound of Table 1 or Table 2, or a pharmaceutically acceptable salt or solvate thereof) as provided herein or a pharmaceutical composition thereof is formulated for local delivery to the digestive or GI tract by way of oral administration (e.g., solid or liquid dosage forms).

In some embodiments, solid dosage forms for oral administration include capsules, tablets, pills, powders, and granules. In some embodiments, a compound disclosed herein, or a pharmaceutically acceptable salt or solvate thereof, (e.g., a compound of Table 1 or Table 2, or a pharmaceutically acceptable salt or solvate thereof) is mixed with one or more pharmaceutically acceptable excipients, such as sodium citrate or dicalcium phosphate and/or: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. For example, in the case of capsules, tablets and pills, the dosage form may also comprise buffering agents. In some embodiments, solid compositions of a similar type may also be employed as fillers in soft and hard-filled gelatin capsules using such excipients as lactose or milk sugar as well as high molecular weight polyethylene glycols and the like.

In some embodiments, the pharmaceutical compositions will take the form of a unit dosage form such as a pill or tablet and thus the composition may contain, along with a compound disclosed herein (e.g., a compound of Table 1 or Table 2, or a pharmaceutically acceptable salt or solvate thereof) as provided herein, a diluent such as lactose, sucrose, dicalcium phosphate, or the like; a lubricant such as magnesium stearate or the like; and a binder such as starch, gum acacia, polyvinylpyrrolidine, gelatin, cellulose, cellulose derivatives or the like. In some embodiments, another solid dosage form, a powder, marume, solution or suspension (e.g., in propylene carbonate, vegetable oils, PEG's, poloxamer 124 or triglycerides) is encapsulated in a capsule (gelatin or cellulose base capsule). In some embodiments, unit dosage forms in which one or more compounds and pharmaceutical compositions as provided herein or additional active agents are physically separated are also contemplated; e.g., capsules with granules (or tablets in a capsule) of each drug; two-layer tablets; two-compartment gel caps, etc. In some embodiments, enteric coated or delayed release oral dosage forms are also contemplated.

In some embodiments, other physiologically acceptable compounds may include wetting agents, emulsifying agents, dispersing agents or preservatives that are particularly useful for preventing the growth or action of microorganisms. For example, various preservatives are well known and include, for example, phenol and ascorbic acid.

In some embodiments, the excipients are sterile and generally free of undesirable matter. For example, these compositions can be sterilized by conventional, well-known sterilization techniques. In some embodiments, for various oral dosage form excipients such as tablets and capsules, sterility is not required. For example, the United States Pharmacopeia/National Formulary (USP/NF) standard can be sufficient.

In some embodiments, a compound disclosed herein (e.g., a compound of Table 1 or Table 2, or a pharmaceutically acceptable salt or solvate thereof) as provided herein or a pharmaceutical composition thereof is formulated for ocular administration. In some embodiments, ocular compositions can include, without limitation, one or more of viscogens (e.g., carboxymethylcellulose, glycerin, polyvinylpyrrolidone, polyethylene glycol); stabilizers (e.g., pluronic (triblock copolymers), cyclodextrins); preservatives (e.g., benzalkonium chloride, ETDA, SofZia (boric acid, propylene glycol, sorbitol, and zinc chloride; Alcon Laboratories, Inc.), Purite (stabilized oxychloro complex; Allergan, Inc.)).

In some embodiments, a compound disclosed herein (e.g., a compound of Table 1 or Table 2, or a pharmaceutically acceptable salt or solvate thereof) as provided herein or a pharmaceutical composition thereof is formulated for topical administration to the skin or mucosa (e.g., dermally or transdermally). In some embodiments, topical compositions can include ointments and creams. In some embodiments, ointments are semisolid preparations that are typically based on petrolatum or other petroleum derivatives. In some embodiments, creams containing the selected active agent are typically viscous liquid or semisolid emulsions, often either oil-in-water or water-in-oil. For example, cream bases are typically water-washable, and contain an oil phase, an emulsifier, and an aqueous phase. For example, the oil phase, also sometimes called the “internal” phase, is generally comprised of petrolatum and a fatty alcohol such as cetyl or stearyl alcohol; the aqueous phase usually, although not necessarily, exceeds the oil phase in volume, and generally contains a humectant. In some embodiments, the emulsifier in a cream formulation is generally a nonionic, anionic, cationic or amphoteric surfactant. In some embodiments, as with other carriers or vehicles, an ointment base should be inert, stable, nonirritating, and non-sensitizing.

In any of the foregoing embodiments, pharmaceutical compositions as provided herein can include one or more one or more of lipids, interbilayer crosslinked multilamellar vesicles, biodegradable poly(D,L-lactic-co-glycolic acid) [PLGA]-based or poly anhydride-based nanoparticles or microparticles, and nanoporous particle-supported lipid bilayers.

In some embodiments, the dosage for a compound disclosed herein (e.g., a compound of Table 1 or Table 2, or a pharmaceutically acceptable salt or solvate thereof), is determined based on a multiple factors including, but not limited to, type, age, weight, sex, medical condition of the subject, severity of the medical condition of the subject, route of administration, and activity of the compound or pharmaceutically acceptable salt or solvate thereof. In some embodiments, proper dosage for a particular situation can be determined by one skilled in the medical arts. In some embodiments, the total daily dosage may be divided and administered in portions throughout the day or by means providing continuous delivery.

In some embodiments, a compound disclosed herein (e.g., a compound of Table 1 or Table 2, or a pharmaceutically acceptable salt or solvate thereof), is administered at a dose from about 0.01 to about 1000 mg. For example, from about 0.1 to about 30 mg, about 10 to about 80 mg, about 0.5 to about 15 mg, about 50 mg to about 200 mg, about 100 mg to about 300 mg, about 200 to about 400 mg, about 300 mg to about 500 mg, about 400 mg to about 600 mg, about 500 mg to about 800 mg, about 600 mg to about 900 mg, or about 700 mg to about 1000 mg. In some embodiments, the dose is a therapeutically effective amount.

In some embodiments, a compound disclosed herein (e.g., a compound of Table 1 or Table 2, or a pharmaceutically acceptable salt or solvate thereof) as provided herein is administered at a dosage of from about 0.0002 mg/Kg to about 100 mg/Kg (e.g., from about 0.0002 mg/Kg to about 50 mg/Kg; from about 0.0002 mg/Kg to about 25 mg/Kg; from about 0.0002 mg/Kg to about 10 mg/Kg; from about 0.0002 mg/Kg to about 5 mg/Kg; from about 0.0002 mg/Kg to about 1 mg/Kg; from about 0.0002 mg/Kg to about 0.5 mg/Kg; from about 0.0002 mg/Kg to about 0.1 mg/Kg; from about 0.001 mg/Kg to about 50 mg/Kg; from about 0.001 mg/Kg to about 25 mg/Kg; from about 0.001 mg/Kg to about 10 mg/Kg; from about 0.001 mg/Kg to about 5 mg/Kg; from about 0.001 mg/Kg to about 1 mg/Kg; from about 0.001 mg/Kg to about 0.5 mg/Kg; from about 0.001 mg/Kg to about 0.1 mg/Kg; from about 0.01 mg/Kg to about 50 mg/Kg; from about 0.01 mg/Kg to about 25 mg/Kg; from about 0.01 mg/Kg to about 10 mg/Kg; from about 0.01 mg/Kg to about 5 mg/Kg; from about 0.01 mg/Kg to about 1 mg/Kg; from about 0.01 mg/Kg to about 0.5 mg/Kg; from about 0.01 mg/Kg to about 0.1 mg/Kg; from about 0.1 mg/Kg to about 50 mg/Kg; from about 0.1 mg/Kg to about 25 mg/Kg; from about 0.1 mg/Kg to about 10 mg/Kg; from about 0.1 mg/Kg to about 5 mg/Kg; from about 0.1 mg/Kg to about 1 mg/Kg; from about 0.1 mg/Kg to about 0.5 mg/Kg). In some embodiments, a compound disclosed herein (e.g., a compound of Table 1 or Table 2, or a pharmaceutically acceptable salt or solvate thereof) as provided herein is administered as a dosage of about 100 mg/Kg.

In some embodiments, the foregoing dosages of a compound disclosed herein (e.g., a compound of Table 1 or Table 2, or a pharmaceutically acceptable salt or solvate thereof), can be administered on a daily basis (e.g., as a single dose or as two or more divided doses) or non-daily basis (e.g., every other day, every two days, every three days, once weekly, twice weeks, once every two weeks, once a month).

In some embodiments, the period of administration of a compound disclosed herein (e.g., a compound of Table 1 or Table 2, or a pharmaceutically acceptable salt or solvate thereof) as provided herein is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In some embodiments, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In some embodiments, a compound disclosed herein (e.g., a compound of Table 1 or Table 2, or a pharmaceutically acceptable salt or solvate thereof) is administered to a subject for a period followed by a separate period of time where administration of a compound disclosed herein, or a pharmaceutically acceptable salt or solvate thereof. (e.g., a compound of Table 1 or Table 2, or a pharmaceutically acceptable salt or solvate thereof) is stopped. In some embodiments, a compound disclosed herein (e.g., a compound of Table 1 or Table 2, or a pharmaceutically acceptable salt or solvate thereof) is administered for a first period and a second period following the first period, with administration stopped during the second period, followed by a third period where administration of a compound disclosed herein, or a pharmaceutically acceptable salt or solvate thereof, (e.g., a compound of Table 1 or Table 2, or a pharmaceutically acceptable salt or solvate thereof) is started and then a fourth period following the third period where administration is stopped. For example, the period of administration of a compound disclosed herein (e.g., a compound of Table 1 or Table 2, or a pharmaceutically acceptable salt or solvate thereof) followed by a period where administration is stopped is repeated for a determined or undetermined period. In some embodiments, a period of administration is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In some embodiments, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more.

In some embodiments, a compound disclosed herein (e.g., a compound of Table 1 or Table 2, or a pharmaceutically acceptable salt or solvate thereof), is orally administered to the subject one or more times per day (e.g., one time per day, two times per day, three times per day, four times per day per day or a single daily dose).

In some embodiments, a compound disclosed herein (e.g., a compound of Table 1 or Table 2, or a pharmaceutically acceptable salt or solvate thereof), is administered by parenteral administration to the subject one or more times per day (e.g., 1 to 4 times one time per day, two times per day, three times per day, four times per day or a single daily dose).

In some embodiments, a compound disclosed herein (e.g., a compound of Table 1 or Table 2, or a pharmaceutically acceptable salt or solvate thereof), is administered by parenteral administration to the subject weekly.

### Synthesis of the Compounds

The compounds of this disclosure can be prepared from readily available starting materials using, for example, the following general methods, and procedures. It will be appreciated that where certain process conditions (i.e., reaction temperatures, times, mole ratios of reactants, solvents, pressures, etc.) are given, other process conditions can also be used unless otherwise stated. Optimum reaction conditions may vary with the reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. Suitable protecting groups for various functional groups as well as suitable conditions for protecting and deprotecting certain functional groups are well known in the art. For example, numerous protecting groups are described in T. W. Greene and G. M. Wuts (1999) Protecting Groups in Organic Synthesis, 3rd Edition, Wiley, New York, and references cited therein.

Furthermore, the compounds of this disclosure may contain one or more chiral centers. Accordingly, if desired, such compounds can be prepared or isolated as pure stereoisomers, i.e., as individual enantiomers or diastereomers, or as stereoisomer-enriched mixtures. All such stereoisomers (and enriched mixtures) are included within the scope of this disclosure, unless otherwise indicated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, chiral resolving agents, and the like.

The starting materials for the following reactions are generally known compounds or can be prepared by known procedures or obvious modifications thereof. For example, many of the starting materials are available from commercial suppliers such as Aldrich Chemical Co. (Milwaukee, Wisconsin, USA), Bachem (Torrance CA USA), EMKA-Chemie Gmbh & Co. KG (Eching Germany), or Millipore Sigma (Burlington MA USA). Others may be prepared by procedures, or obvious modifications thereof, described in standard reference texts such as Fieser and Fieser's Reagents for Organic Synthesis, Volumes 1-15 (John Wiley, and Sons, 1991), Rodd's Chemistry of Carbon Compounds, Volumes 1-5, and Supplementals (Elsevier Science Publishers, 1989), Organic Reactions. Volumes 1-40 (John Wiley. and Sons, 1991), March's Advanced Organic Chemistry, (John Wiley, and Sons, 5^{th} Edition, 2001), and Larock's Comprehensive Organic Transformations (VCH Publishers Inc., 1989).

### General Synthesis

Typical embodiments of compounds described herein may be synthesized using the general reaction schemes described below. It will be apparent given the description herein that the general schemes may be altered by substitution of the starting materials with other materials having similar structures to result in products that are correspondingly different. Descriptions of syntheses follow to provide numerous examples of how the starting materials may vary to provide corresponding products. Given a desired product for which the substituent groups are defined, the necessary starting materials generally may be determined by inspection. Starting materials are typically obtained from commercial sources or synthesized using published methods. For synthesizing compounds which are embodiments described in the present disclosure, inspection of the structure of the compound to be synthesized will provide the identity of each substituent group. The identity of the final product will generally render apparent the identity of the necessary starting materials by a simple process of inspection, given the examples herein. In general, compounds described herein are typically stable and isolatable at room temperature and pressure.

### EXAMPLES

The following examples are included to demonstrate specific embodiments of the disclosure. It should be appreciated by those of skill in the art that the techniques disclosed in the examples which follow represent techniques to function well in the practice of the disclosure, and thus can be considered to constitute specific modes for its practice. However, those of skill in the art should, in light of the present disclosure, appreciate that many changes can be made in the specific embodiments which are disclosed and still obtain a like or similar result without departing from the spirit and scope of the disclosure.

### Abbreviations (as Used Herein)

| | |
|---|---|
| AcOH | acetic acid |
| AIBN | azobisisobutyronitrile |
| aq. | aqueous |
| CCl₄ | carbon tetrachloride |
| CH₃CN | acetonitrile |
| CH₂Cl₂ or DCM | dichloromethane |
| DBU | 1,8-diazabicyclo[5.4.0]undec-7-ene |
| DIAD | diisopropyl azodicarboxylate |
| DIBAL-H | diisobutylaluminium hydride |
| DIEA or DIPEA | N-ethyl-N,N-diisopropylamine |
| DMAP | 4-(dimethylamino)pyridine |
| DMF | N,N-dimethyl-formamide |
| DMSO | Dimethyl sulfoxide |
| EtOAc | ethyl acetate |
| EtOH | ethanol |
| Et₃N | triethylamine |
| h | hour |
| HCl | hydrochloric acid |
| H₂O | water |
| HPLC | High Performance Liquid Chromatography |
| [Ir(COD)(OMe)]₂ | (1,5-cyclooctadiene)(methoxy)iridium(l) dimer |
| K₂CO₃ | potassium carbonate |
| K₂OsO₄•2H₂O | potassium osmate |
| LiBH₄ | lithium borohydride |
| MeI | iodomethane |
| MeOH | methanol |
| MsCl | methanesulfonyl chloride |
| Na₂CO₃ | sodium carbonate |
| NaHCO₃ | sodium bicarbonate |
| NaIO₄ | sodium periodate |
| NaCN | sodium cyanide |
| Na₂SO₄ | sodium sulfate |
| NaH | sodium hydride |
| NaH₂PO₄ | sodium phosphate monobasic |
| NaOH | sodium hydroxide |
| NBS | bromosuccinimide |
| n-BuLi | n-Butyllithium |
| NH₄Cl | ammonium chloride |
| NMP | 1-methyl-pyrrolidin-2-one |
| Pd₂(dba)₃ | tris(dibenzylideneacetone)dipalladium |
| Pd(dppf)Cl₂•CH₂Cl₂ | [1,1′-bis(diphenylphosphino)- |
| | ferrocene]palladium(II) chloride- |
| | dichlormethane complex |
| Pd(OAc)₂ | palladium (II) Acetate |
| PE | Petrol ether |
| PPh₃ | triphenylphosphine |
| Prep. | preparative |
| PTSA | p-toluenesulfonic acid |
| sat. | saturated |
| s-Phos | dicyclohexyl-(2′,6′- |
| | dimethoxybiphenyl-2-yl)-phosphane |
| t-BuOK | potassium tert-butoxide |
| t-BuXphos | di-tert-butyl(2′,4′,6′-triisopropyl- |
| | [1,1′-biphenyl]-2-yl)phosphine |
| TBAF | tetrabutylammonium fluoride |
| TBS | tert-butyldimethylsilyl |
| TMSCI | chlorotrimethylsilane |
| TFA | trifluoroacetic acid |
| THF | tetrahydrofuran |
| TLC | Thin Layer Chromatography |
| Xantphos | 4,5-bis(diphenylphosphino)-9,9-dimethylxanthene |
| Xphos | dicyclohexyl(2′,4′,6′-triisopropyl- |
| | [1,1′-biphenyl]-2-yl)phosphane |

General information: All evaporations or concentrations were carried out in vacuo with a rotary evaporator. Analytical samples were dried in vacuo (1-5 mmHg) at rt. Thin layer chromatography (TLC) was performed on silica gel plates, spots were visualized by UV light (214 and 254 nm). Purification by column and flash chromatography was carried out using silica gel (100-200 mesh). Solvent systems were reported as mixtures by volume. NMR spectra were recorded on a Bruker 400 or Varian (400 MHz) spectrometer. ¹H chemical shifts are reported in S values in ppm with the deuterated solvent as the internal standard. Data are reported as follows: chemical shift, multiplicity (s=singlet, d=doublet, t=triplet, q=quartet, br=broad, m=multiplet), coupling constant (Hz), integration. LCMS spectra were obtained on SHIMADZU LC20-MS2020 or Agilent 1260 series 6125B mass spectrometer or Agilent 1200 series, 6110 or 6120 mass spectrometers with electrospray ionization and excepted as otherwise indicated.

### Example A1

### 2-((2S,3R)-2-(cyclopentyloxy)-3-hydroxy-3-(3-methoxy-4-methylphenyl)propyl)-6-ethoxybenzo[d]thiazole-4-carboxylic acid (Compound 101)

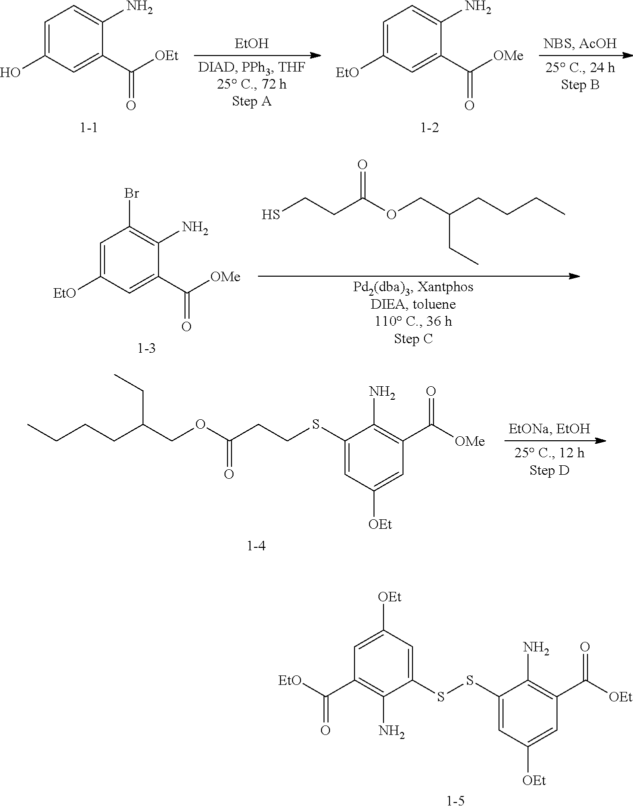

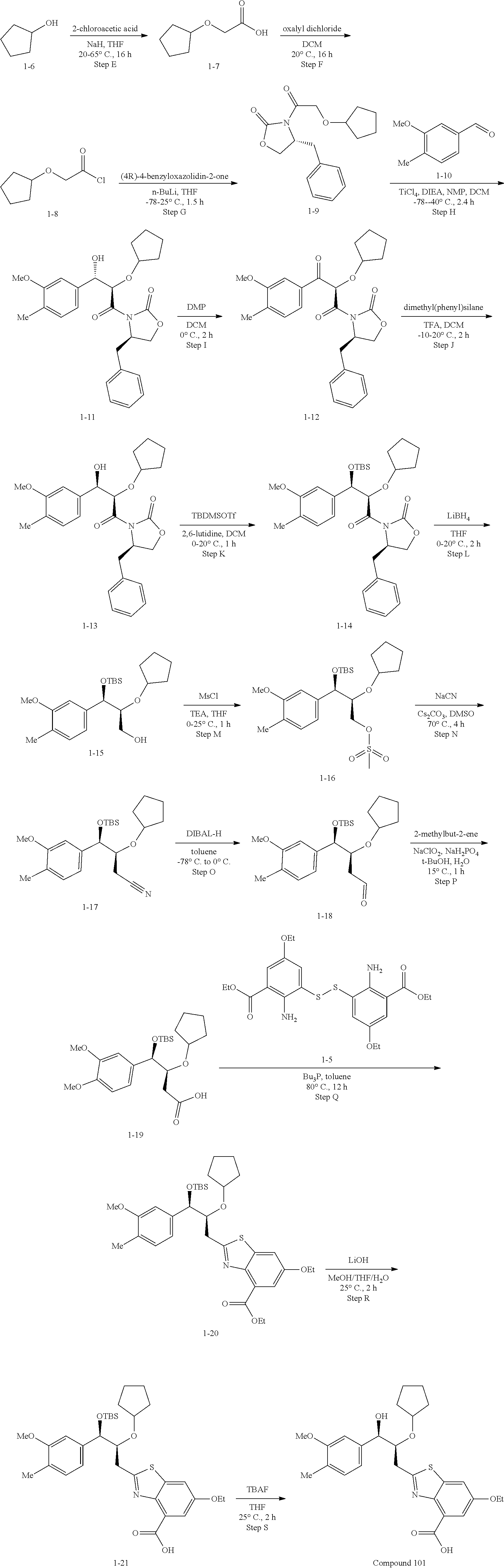

### Step A: methyl 2-amino-5-ethoxybenzoate

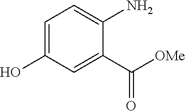 [SMILES: COC(=O)c1cc(O)ccc1N]

To the solution of methyl 2-amino-5-hydroxy-benzoate (10.0 g, 59.8 mmol), EtOH (5.51 g, 6.99 mL) and PPh₃ (31.4 g, 120 mmol) in THF (150 mL) was added DIAD (24.2 g, 23.3 mL) in dropwise at 25° C. The resulting reaction mixture was stirred at 25° C. for 72 h. The solvent was removed under reduced pressure to give a residue. The residue was purified by flash silica gel chromatography (ISCO®; 120 g SepaFlash® Silica Flash Column, Eluent of 0˜15% EtOAc/PE gradient @ 100 mL/min) to afford methyl 2-amino-5-ethoxy-benzoate (10.9 g, 93.0% yield). ¹H NMR (400 MHz, DMSO-d₆) δ 7.17 (d, J=2.8 Hz, 1H), 6.97 (dd, J=8.8, 3.2 Hz, 1H), 6.74 (d, J=8.8 Hz, 1H), 6.29 (s, 2H), 3.89 (q, J=6.8 Hz, 2H), 3.78 (s, 3H), 1.27 (t, J=7.0 Hz, 3H).

### Step B: methyl 2-amino-3-bromo-5-ethoxybenzoate

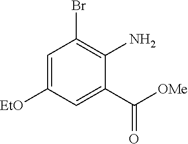 [SMILES: CCOc1cc(Br)c(N)c(C(=O)OC)c1]

To the solution of methyl 2-amino-5-ethoxy-benzoate (10.9 g, 55.6 mmol) in AcOH (80 mL) was added NBS (9.90 g, 55.6 mmol). The resulting mixture was stirred at 25° C. for 24 h. The reaction mixture was poured into water (60 mL) and stirred for 10 min. The aqueous phase was extracted with EtOAc (30 mL×3). The combined organic layers were dried with anhydrous Na₂SO₄, filtered and concentrated in vacuum. The residue was purified by flash silica gel chromatography (ISCO®; 120 g SepaFlash® Silica Flash Column, Eluent of 0˜5% EtOAc/PE gradient @ 100 mL/min) to give methyl 2-amino-3-bromo-5-ethoxy-benzoate (6.65 g, 43.7% yield). ¹H NMR (400 MHz, CDCl₃) δ 7.32 (d, J=2.8 Hz, 1H), 7.20 (d, J=2.8 Hz, 1H), 5.59 (brs, 2H), 3.87 (q, J=7.0 Hz, 2H), 3.80 (s, 3H), 1.29 (t, J=7.0 Hz, 3H).

### Step C: methyl 2-amino-5-ethoxy-3-((3-((2-ethylhexyl)oxy)-3-oxopropyl)thio)benzoate

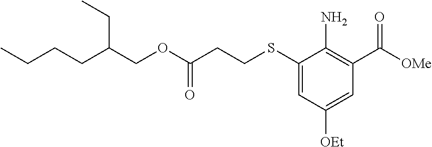 [SMILES: CCCCC(CC)COC(=O)CCSc1cc(OCC)cc(C(=O)OC)c1N]

To the solution of 2-ethylhexyl 3-sulfanylpropanoate (5.30 g, 24.3 mmol) and methyl 2-amino-3-bromo-5-ethoxy-benzoate (6.65 g, 24.3 mmol) in toluene (80 mL) was added Pd₂(dba)₃ (1.11 g, 1.21 mmol), Xantphos (1.40 g, 2.43 mmol) and DIEA (7.84 g, 60.7 mmol, 10.6 mL). The mixture was stirred at 110° C. for 36 h. The residue was poured into water (150 mL) and stirred for 10 min. The aqueous phase was extracted with EtOAc (80 mL×3). The combined organic layers were dried with anhydrous Na₂SO₄, filtered and concentrated in vacuum. The residue was purified by flash silica gel chromatography (ISCO®; 120 g SepaFlash® Silica Flash Column, Eluent of 0˜5% EtOAc/PE gradient @ 100 mL/min) to afford methyl 2-amino-5-ethoxy-3-[3-(2-ethylhexoxy)-3-oxo-propyl] sulfanyl-benzoate (8.29 g, 83.0% yield).

### Step D: methyl 2-amino-5-ethoxy-3-[(3-ethoxy-5-methoxycarbonyl-phenyl)disulfanyl]benzoate

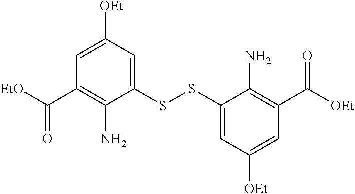 [SMILES: CCOC(=O)c1cc(OCC)cc(SSc2cc(OCC)cc(C(=O)OCC)c2N)c1N]

To the solution of methyl 2-amino-5-ethoxy-3-[2-(2-ethylhexoxy)-2-oxo-ethyl]sulfanyl-benzoate (8.29 g, 20.9 mmol) in EtOH (50 mL) was added EtONa (9.22 g, 27.1 mmol, 20% in EtOH). The reaction mixture was stirred at 25° C. for 12 h. The residue was diluted with water (100 mL), acidified to pH=6 with 37% HCl, extracted with EtOAc (100 mL×3). The combined organic layers were dried with anhydrous Na₂SO₄, filtered and concentrated in vacuum. The residue was purified by flash silica gel chomatography (ISCO®; 120 g SepaFlash® Silica Flash Column, Eluent of 0˜5% EtOAc/PE gradient @ 100 ml/min) to give methyl 2-amino-5-ethoxy-3-[(3-ethoxy-5-methoxycarbonyl-phenyl)disulfanyl]benzoate (1.09 g, 12.0% yield). ¹H NMR (400 MHz, CDCl₃) δ 7.46 (d, J=3.2 Hz, 2H), 6.84 (d, J=3.2 Hz, 2H), 6.37 (brs, 4H), 4.27 (q, J=7.2 Hz, 4H), 3.75 (q, J=6.8 Hz, 4H), 1.32 (t, J=7.0 Hz, 6H), 1.24 (t, J=7.0 Hz, 6H).

### Step E: 2-(cyclopentyloxy)acetic acid

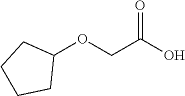

To a solution of cyclopentanol (3 g, 34.83 mmol) in THF (50 mL) was added NaH (2.79 g, 69.66 mmol, 60% purity) at 0° C. After being stirred at 60° C. for 30 min, then 2-chloroacetic acid (3.29 g, 34.83 mmol) was added slowly to the reaction mixture at 25° C. The resulting mixture was stirred at 60° C. for 16 h. After cooling, the reaction mixture was quenched with H₂O (10 mL), diluted with H₂O (50 mL), the pH value was adjusted to 5 with 1N HCl. The mixture was extracted with EtOAc (40 mL×3). The combined organic layers were washed with brine (50 mL×3), dried over anhydrous Na₂SO₄, filtered and concentrated under reduced pressure to give a residue. The residue was purified by silica gel column chromatography (PE/EtOAc=1/0 to 1/1) to afford 2-(cyclopentoxy)acetic acid (3.7 g, 73% yield). ¹H NMR (400 MHz, DMSO-d6) δ 4.21-4.19 (m, 1H), 3.94 (s, 2H), 1.71-1.28 (m, 8H).

### Step F: 2-(cyclopentyloxy)acetyl chloride

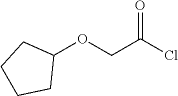

To a solution of 2-(cyclopentoxy)acetic acid (3.7 g, 25.66 mmol) in DCM (20 mL) was added oxalyl dichloride (4.89 g, 38.50 mmol) and DMF (0.1 mL). Then the mixture was stirred at 25° C. for 1 h. The reaction mixture was concentrated under reduced pressure to afford 2-(cyclopentoxy)acetyl chloride (4.17 g, crude), which was used directly for the next step without further purification.

### Step G: (R)-4-benzyl-3-(2-(cyclopentyloxy)acetyl)oxazolidin-2-one

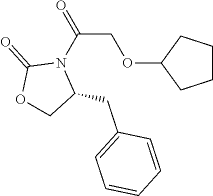 [SMILES: O=C(COC1CCCC1)N1C(=O)OC[C@H]1Cc1ccccc1]

To a solution of (4R)-4-benzyloxazolidin-2-one (6.82 g, 38.47 mmol) in THF (30 mL) was added n-BuLi (14.36 mL, 35.9 mmol, 2.5 M solution in Hexanes) at −78° C. in dropwise under N₂. After addition, a solution of 2-(cyclopentoxy)acetyl chloride (4.17 g, 25.64 mmol) in THF (8 mL) was added to the mixture at −78° C. The resulting mixture was stirred at 25° C. for 2 h. Then the reaction mixture was quenched by sat. aq. NH₄Cl solution (50 mL), extracted with EtOAc (30 mL×3). The combined organic layers were washed with water (20 mL×3), dried over anhydrous Na₂SO₄, filtered and concentrated under reduced pressure to give a residue. The residue was purified by silica gel column chromatography (PE/EtOAc=1/0 to 1/1) to afford (4R)-4-benzyl-3-[2-(cyclopentoxy)acetyl]oxazolidin-2-one (2.14 g, 23% yield). LC-MS: m/z 304.1 (M+H)⁺.

### Step H: (R)-4-benzyl-3-((2R,3S)-2-(cyclopentyloxy)-3-hydroxy-3-(3-methoxy-4-methylphenyl)propanoyl)oxazolidin-2-one

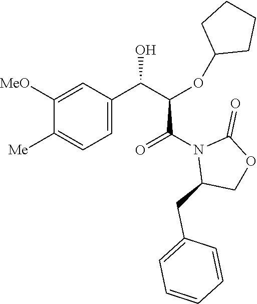 [SMILES: COc1cc([C@H](O)[C@@H](OC2CCCC2)C(=O)N2C(=O)OC[C@H]2Cc2ccccc2)ccc1C]

To a solution of (R)-4-benzyl-3-(2-(cyclopentyloxy)acetyl)oxazolidin-2-one (1.8 g, 5.93 mmol) in DCM (45 mL) was added TiCl₄ (683 μL, 6.23 mmol) at −78° C. under nitrogen. The mixture was stirred at −78° C. for 15 minutes. Then DIEA (2.58 mL, 14.8 mmol) was added in dropwise at −78° C. The resulting mixture was stirred at −78° C. for 40 minutes. Then NMP (577 μL, 5.93 mmol) was added in dropwise. The reaction mixture was stirred at −78° C. for 10 mins under nitrogen. Then 3-methoxy-4-methylbenzaldehyde (980 mg, 6.53 mmol) in dry DCM (10 mL) was added in dropwise. The resulting mixture was stirred at −78° C. for 2 h under nitrogen. Then the reaction mixture was quenched with sat. aq. NH₄Cl (50 mL), extracted with DCM (60 mL×3). The organic layers were dried over anhydrous Na₂SO₄, filtered and concentrated to get a residue. The residue was purified by flash silica gel chromatography (ISCO®; 40 g Sepa Flash® Silica Flash Column, Eluent of 0˜45% EtOAc/PE gradient @,& 40 mL/min) to give (R)-4-benzyl-3-((2R,3S)-2-(cyclopentyloxy)-3-hydroxy-3-(3-methoxy-4-methylphenyl)propanoyl)oxazolidin-2-one (2.27 g, 84.4% yield). LC-MS: m/z 476.2 (M+Na)⁺.

### Step I: (R)-1-((R)-4-benzyl-2-oxooxazolidin-3-yl)-2-(cyclopentyloxy)-3-(3-methoxy-4-methylphenyl)propane-1,3-dione

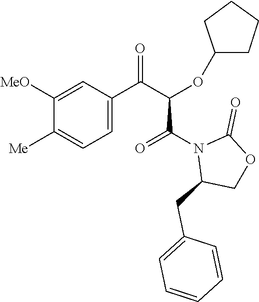 [SMILES: COc1cc(C(=O)[C@@H](OC2CCCC2)C(=O)N2C(=O)OC[C@H]2Cc2ccccc2)ccc1C]

At 0° C., to the mixture of (R)-4-benzyl-3-((2R,3S)-2-(cyclopentyloxy)-3-hydroxy-3-(3-methoxy-4-methylphenyl)propanoyl)oxazolidin-2-one (2.27 g, 5.01 mmol) in DCM (30 mL) was added Dess-Martin periodinane (4.25 g, 10.0 mmol) in portions. The reaction mixture was stirred at 0° C. for 2 h. Then the mixture was quenched with H₂O (50 mL) and DCM (50 mL). The mixture was filtered through a celite and extracted with DCM (50 mL×2). The organic layers were dried over Na₂SO₄, filtered and concentrated under vacuum. The residue was purified by flash silica gel chromatography (ISCO®; 40 g Sepa Flash® Silica Flash Column, Eluent of 0˜35% EtOAc/PE gradient (a 40 mL/min) to give (R)-1-((R)-4-benzyl-2-oxooxazolidin-3-yl)-2-(cyclopentyloxy)-3-(3-methoxy-4-methylphenyl)propane-1,3-dione (2.2 g, 97.4% yield). LC-MS: m/z 474.1 (M+Na)⁺.

### Step J: (R)-4-benzyl-3-((2R,3R)-3-((tert-butyldimethylsilyl)oxy)-2-(cyclopentyloxy)-3-(3-methoxy-4-methylphenyl)propanoyl)oxazolidin-2-one

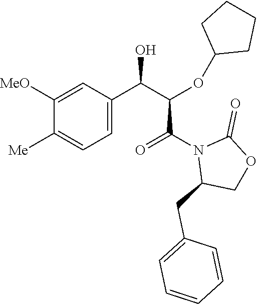 [SMILES: COc1cc([C@@H](O)[C@@H](OC2CCCC2)C(=O)N2C(=O)OC[C@H]2Cc2ccccc2)ccc1C]

At −10° C., to the mixture of (R)-1-((R)-4-benzyl-2-oxooxazolidin-3-yl)-2-(cyclopentyloxy)-3-(3-methoxy-4-methylphenyl)propane-1,3-dione (2.2 g, 4.87 mmol) in TFA (21.7 mL) and DCM (22 mL) was added dimethyl(phenyl)silane (2.27 mL, 14.6 mmol) in dropwise. The reaction mixture was stirred at −10° C. for 2 h. The solution was poured into sat. aq. NaHCO₃ (200 mL), extracted with DCM (30 mL×3). The combined organics were dried over anhydrous Na₂SO₄, filtered and concentrated. The residue was purified by flash silica gel chromatography (ISCO®; 40 g Sepa Flash® Silica Flash Column, Eluent of 0˜50% EtOAc/PE gradient @ 40 mL/min) to give (R)-4-benzyl-3-((2R,3R)-2-(cyclopentyloxy)-3-hydroxy-3-(3-methoxy-4-methylphenyl)propanoyl)oxazolidin-2-one (1.85 g, 83.7% yield). LC-MS: m/z 476.1 (M+Na)⁺.

### Step K: (R)-4-benzyl-3-((2R,3R)-3-((tert-butyldimethylsilyl)oxy)-2-(cyclopentyloxy)-3-(3-methoxy-4-methylphenyl)propanoyl)oxazolidin-2-one

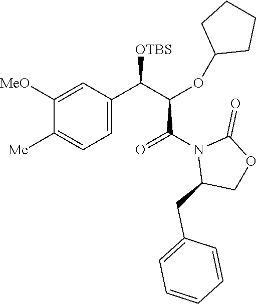 [SMILES: COc1cc([C@@H](O[Si](C)(C)C(C)(C)C)[C@@H](OC2CCCC2)C(=O)N2C(=O)OC[C@H]2Cc2ccccc2)ccc1C]

To the mixture of (R)-4-benzyl-3-((2R,3R)-2-(cyclopentyloxy)-3-hydroxy-3-(3-methoxy-4-methylphenyl)propanoyl)oxazolidin-2-one (1.85 g, 4.08 mmol) in DCM (15 mL) and 2,6-dimethylpyridine (950 μL, 8.16 mmol) was added [tert-butyl(dimethyl)silyl] trifluoromethanesulfonate (1.88 mL, 8.16 mmol) at 0° C. The reaction mixture was stirred at 0° C. for 2 h. Then the mixture was quenched with H₂O (50 mL), extracted with DCM (50 mL×3). The organic layers were washed with H₂O (50 mL×2), dried over anhydrous Na₂SO₄ and concentrated under vacuum. The residue was purified by flash silica gel chromatography (ISCO®; 20 g Sepa Flash® Silica Flash Column, Eluent of 0˜10% EtOAc/PE gradient @ 35 mL/min) to give (4R)-4-benzyl-3-[(2S,3R)-3-[tert-butyl(dimethyl)silyl]oxy-2-(cyclopentoxy)-3-(3-methoxy-4-methyl-phenyl)propanoyl]oxazolidin-2-one (2.2 g, 95.0% yield).

### Step L: (2S,3R)-3-((tert-butyldimethylsilyl)oxy)-2-(cyclopentyloxy)-3-(3-methoxy-4-methylphenyl)propan-1-ol

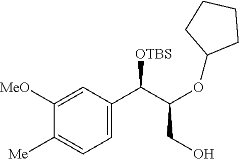 [SMILES: COc1cc([C@@H](O[Si](C)(C)C(C)(C)C)[C@H](CO)OC2CCCC2)ccc1C]

At 0° C., to the mixture of LiBH₄ (9.69 mL, 38.76 mmol, 4 M solution in THF) in THF (10 mL) was added H₂O (15.4 mg, 852 μmol) dropwise under N₂. The mixture was stirred at 0° C. for 0.5 h. Then a solution of (R)-4-benzyl-3-((2R,3R)-3-((tert-butyldimethylsilyl)oxy)-2-(cyclopentyloxy)-3-(3-methoxy-4-methylphenyl)propanoyl)oxazolidin-2-one (2.2 g, 3.87 mmol) in THF (20 mL) was added in dropwise. The reaction mixture was warmed up to 15° C. and stirred at 15° C. for 16 h. The reaction mixture was carefully neutralized with 1 M aq. HCl solution, extracted with EtOAc (30 mL×3). The organic layers were washed with brine (50 mL), dried over anhydrous Na₂SO₄, filtered and concentrated. The residue was purified by flash silica gel chromatography (ISCO®; 40 g Sepa Flash® Silica Flash Column, Eluent of 0˜10% EtOAc/PE gradient @,& 40 mL/min) to give (2S,3R)-3-((tert-butyldimethylsilyl)oxy)-2-(cyclopentyloxy)-3-(3-methoxy-4-methylphenyl)propan-1-ol (1.26 g, 82.4% yield). ¹H NMR (400 MHz, CDCl₃) δ 7.04 (d, J=7.2 Hz, 1H), 6.85 (s, 1H), 6.79 (d, J=7.6 Hz, 1H), 4.61 (d, J=6.8 Hz, 1H), 3.82 (s, 3H), 3.81-3.70 (m, 3H), 3.36-3.35 (m, 1H), 2.20 (s, 3H), 1.58-1.26 (m, 8H), 0.89 (s, 9H), 0.05 (s, 3H), −0.16 (s, 3H).

### Step M: (2S,3R)-3-((tert-butyldimethylsilyl)oxy)-2-(cyclopentyloxy)-3-(3-methoxy-4-methylphenyl)propyl methanesulfonate

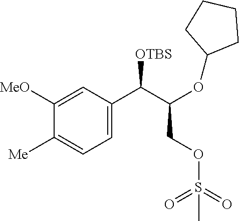 [SMILES: COc1cc([C@@H](O[Si](C)(C)C(C)(C)C)[C@H](COS(C)(=O)=O)OC2CCCC2)ccc1C]

To the mixture of (2S,3R)-3-((tert-butyldimethylsilyl)oxy)-2-(cyclopentyloxy)-3-(3-methoxy-4-methylphenyl)propan-1-ol (1.26 g, 3.19 mmol) in DCM (20 mL) and TEA (667 μL, 4.79 mmol) was added MsCl (439 mg, 3.83 mmol, 297 μL) in dropwise at 0° C. The reaction mixture was stirred at 0° C. for 0.5 h. The mixture was quenched with sat. aq. NaHCO₃ (50 mL), extracted with DCM (20 mL×3). The organic layers were washed with 0.5N HCl (20 mL×2), brine (20 mL×2), dried over Na₂SO₄ and concentrated under vacuum. The residue was purified by flash silica gel chromatography (ISCO®; 20 g Sepa Flash® Silica Flash Column, Eluent of 0˜15% EtOAc/PE gradient (@ 35 mL/min) to give (2S,3R)-3-((tert-butyldimethylsilyl)oxy)-2-(cyclopentyloxy)-3-(3-methoxy-4-methylphenyl)propyl methanesulfonate (1.42 g, 94.1% yield). ¹H NMR (400 MHz, CDCl₃) δ 7.05 (d, J=7.6 Hz, 1H), 6.84 (s, 1H), 6.79-6.76 (m, 1H), 4.65 (d, J=5.6 Hz, 1H), 4.41-4.27 (m, 2H), 3.82 (s, 4H), 3.57-3.56 (m, 1H), 2.99 (s, 3H), 2.20 (s, 3H), 1.61-1.30 (m, 8H), 0.89 (s, 9H), 0.07 (s, 3H), −0.14 (s, 3H).

### Step N: (3S,4R)-4-((tert-butyldimethylsilyl)oxy)-3-(cyclopentyloxy)-4-(3-methoxy-4-methylphenyl)butanenitrile

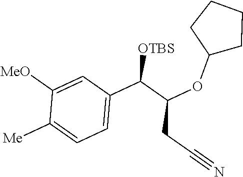 [SMILES: COc1cc([C@@H](O[Si](C)(C)C(C)(C)C)[C@H](CC#N)OC2CCCC2)ccc1C]

To a solution of [(2S,3R)-3-[tert-butyl(dimethyl)silyl]oxy-2-(cyclopentoxy)-3-(3-methoxy-4-methyl-phenyl)propyl] methanesulfonate (6.0 g, 12.69 mmol) in DMSO (60 mL) was added NaCN (3.11 g, 63.46 mmol). The mixture was stirred at 85° C. for 3 h. After cooling, the mixture was quenched with H₂O (60 mL), extracted with EtOAc (60 mL×2). The combined organic layers were washed with brine (50 mL), dried over anhydrous Na₂SO₄, filtered and concentrated. The residue was purified by silica gel column (PE/EtOAc=10/1) to afford (3S,4R)-4-((tert-butyldimethylsilyl)oxy)-3-(cyclopentyloxy)-4-(3-methoxy-4-methylphenyl) butanenitrile (4 g, 78.1% yield). LC-MS: m/z 426.2 (M+Na)⁺.

### Step O: (3S,4R)-4-((tert-butyldimethylsilyl)oxy)-3-(cyclopentyloxy)-4-(3-methoxy-4-methylphenyl)butanal

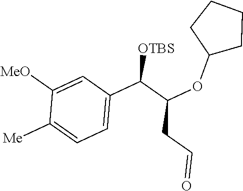 [SMILES: COc1cc([C@@H](O[Si](C)(C)C(C)(C)C)[C@H](CC=O)OC2CCCC2)ccc1C]

To a solution of (3S,4R)-4-[tert-butyl(dimethyl)silyl]oxy-3-(cyclopentoxy)-4-(3-methoxy-4-methyl-phenyl)butanenitrile (4.0 g, 9.91 mmol) in toluene (50 mL) was added DIBAL-H (19.82 mL, 19.82 mmol) at −78° C. The reaction mixture was stirred at 0° C. for 1 h. Then the mixture was quenched with potassium sodium tartaric solution (50 mL), extracted with EtOAc (50 mL×3). The combined organic layers were washed with brine (50 mL), dried over anhydrous Na₂SO₄, filtered and concentrated to afford crude (3S,4R)-4-[tert-butyl(dimethyl)silyl]oxy-3-(cyclopentoxy)-4-(3-methoxy-4-methyl-phenyl)butanal (4.0 g, 99.2% yield), which used for next step without further purification.

### Step P: (3S,4R)-4-((tert-butyldimethylsilyl)oxy)-3-(cyclopentyloxy)-4-(3-methoxy-4-methylphenyl)butanoic acid

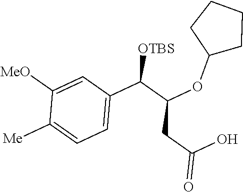 [SMILES: COc1cc([C@@H](O[Si](C)(C)C(C)(C)C)[C@H](CC(=O)O)OC2CCCC2)ccc1C]

To a solution of (3S,4R)-4-[tert-butyl(dimethyl)silyl]oxy-3-(cyclopentoxy)-4-(3-methoxy-4-methyl-phenyl)butanal (4.0 g, 9.84 mmol) in t-BuOH (40 mL) and H₂O (10 mL) was added NaH₂PO₄ (1.18 g, 9.84 mmol), sodium chlorite (3.20 g, 35.41 mmol) and 2-methylbut-2-ene (3.10 g, 44.27 mmol). The reaction mixture was stirred at 25° C. for 1 h. Then the mixture was quenched with H₂O (60 mL), extracted with EtOAc (60 mL×2). The combined organic layers were washed with brine (50 mL), dried over anhydrous Na₂SO₄, filtered and concentrated. The residue was purified by silica gel column (PE/EtOAc=10/1) to afford (3S,4R)-4-[tert-butyl(dimethyl)silyl]oxy-3-(cyclopentoxy)-4-(3-methoxy-4-methyl-phenyl)butanoic acid (1.6 g, 38.5% yield). LC-MS: m/z 445.1 (M+Na)⁺.

### Step Q: ethyl 2-((2S,3R)-3-((tert-butyldimethylsilyl)oxy)-2-(cyclopentyloxy)-3-(3-methoxy-4-methylphenyl)propyl)-6-ethoxybenzo[d]thiazole-4-carboxylate

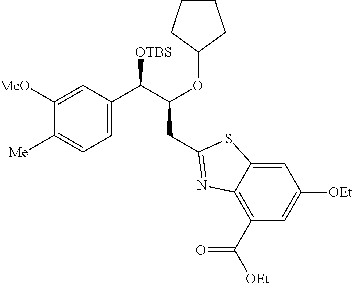 [SMILES: CCOC(=O)c1cc(OCC)cc2sc(C[C@H](OC3CCCC3)[C@H](O[Si](C)(C)C(C)(C)C)c3ccc(C)c(OC)c3)nc12]

To a solution of (3S,4R)-4-[tert-butyl(dimethyl)silyl]oxy-3-(cyclopentoxy)-4-(3-methoxy-4-methyl-phenyl)butanoic acid (300 mg, 709.84 μmol) and ethyl 2-amino-3-[(2-amino-5-ethoxy-3-ethoxycarbonyl-phenyl)disulfanyl]-5-ethoxy-benzoate (341 mg, 709.84 μmol) in toluene (10 mL) was added tributylphosphane (430 mg, 2.13 mmol). The reaction mixture was stirred at 80° C. for 12 h. The mixture was quenched with H₂O (30 mL), extracted with EtOAc (30 mL×3). The combined organic layers were washed with brine (50 mL), dried over anhydrous Na₂SO₄, filtered and concentrated. The residue was purified by silica gel column (PE/EtOAc=10/1) to afford compound ethyl 2-[(2S,3R)-3-[tert-butyl(dimethyl)silyl]oxy-2-(cyclopentoxy)-3-(3-methoxy-4-methyl-phenyl)propyl]-6-ethoxy-1,3-benzothiazole-4-carboxylate (110 mg, 24.7% yield). LC-MS: m/z 628.3 (M+H)⁺.

### Step R: 2-((2S,3R)-3-((tert-butyldimethylsilyl)oxy)-2-(cyclopentyloxy)-3-(3-methoxy-4-methylphenyl)propyl)-6-ethoxybenzo[d]thiazole-4-carboxylic acid

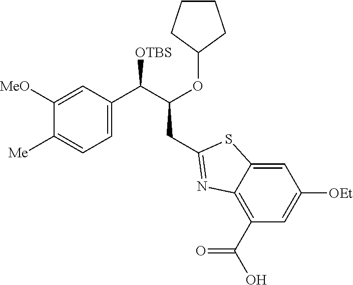 [SMILES: CCOc1cc(C(=O)O)c2nc(C[C@H](OC3CCCC3)[C@H](O[Si](C)(C)C(C)(C)C)c3ccc(C)c(OC)c3)sc2c1]

To a solution of ethyl 2-[(2S,3R)-3-[tert-butyl(dimethyl)silyl]oxy-2-(cyclopentoxy)-3-(3-methoxy-4-methyl-phenyl)propyl]-6-ethoxy-1,3-benzothiazole-4-carboxylate (110 mg, 175.19 μmol) in THF (2 mL), EtOH (2 mL) and H₂O (2 mL) was added LiOH H₂O (37 mg, 875.93 μmol). The mixture was stirred at 25° C. for 1 h. The reaction mixture was adjusted to pH=4 with 1 N HCl, extracted with EtOAc (20 mL×3). The combined organic layers were washed with brine (50 mL), dried over anhydrous Na₂SO₄, filtered and concentrated to afford 2-[(2S,3R)-3-[tert-butyl(dimethyl)silyl]oxy-2-(cyclopentoxy)-3-(3-methoxy-4-methyl-phenyl)propyl]-6-ethoxy-1,3-benzothiazole-4-carboxylic acid (100 mg, 95.1% yield), which used for next step without further purification. LC-MS: m/z 600.3 (M+H)⁺.

### Step S: 2-((2S,3R)-2-(cyclopentyloxy)-3-hydroxy-3-(3-methoxy-4-methylphenyl)propyl)-6-ethoxybenzo[d]thiazole-4-carboxylic acid (Compound 101)

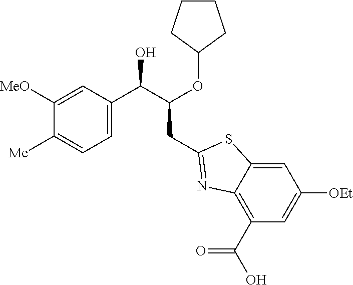 [SMILES: CCOc1cc(C(=O)O)c2nc(C[C@H](OC3CCCC3)[C@H](O)c3ccc(C)c(OC)c3)sc2c1]

To a solution of 2-[(2S,3R)-3-[tert-butyl(dimethyl)silyl]oxy-2-(cyclopentoxy)-3-(3-methoxy-4-methyl-phenyl)propyl]-6-ethoxy-1,3-benzothiazole-4-carboxylic acid (100 mg, 166.71 μmol) in THF (5 mL) was added TBAF (1.67 mL, 1.67 mmol). The mixture was stirred at 25° C. for 1 h. The reaction mixture was diluted with H₂O (30 mL), extracted with EtOAc (30 mL×3). The combined organic layers were washed with brine (50 mL), dried over anhydrous Na₂SO₄, filtered and concentrated. The residue was purified by prep. HPLC (Column: Welch Xtimate C18 150*30 mm*5 μm; Mobile phase A: [water (0.1% HCOOH)], Mobile Phase B: CH₃CN; Gradient: 55% B % to 85% B % in 7 min) to afford 2-[(2S,3R)-2-(cyclopentoxy)-3-hydroxy-3-(3-methoxy-4-methyl-phenyl)propyl]-6-ethoxy-1,3-benzothiazole-4-carboxylic acid (30 mg, 36.5% yield). LC-MS: m/z 486.1 (M+Na). ¹H NMR (400 MHz, CD₃OD) δ 7.74 (d, J=4.0, 1H), 7.71 (d, J=4.0 Hz, 1H), 7.07 (d, J=8.0 Hz, 1H), 6.98 (s, 1H), 6.88 (d, J=8.0 Hz, 1H), 4.69 (d, J=8.0 Hz, 1H), 4.15 (q, J=8.0 Hz, 2H), 3.98-3.88 (m, 2H), 3.84 (s, 3H), 3.43 (d, J=4.0 Hz, 2H), 2.15 (s, 3H), 1.54-1.40 (m, 8H), 1.38-1.28 (m, 3H).

### Example A2

### 2-((2S,3R)-2-(cyclopentyloxy)-3-(4-(difluoromethyl)-3-methoxyphenyl)-3-hydroxypropyl)-6-methoxybenzo[d]thiazole-4-carboxylic acid (Compound 102)

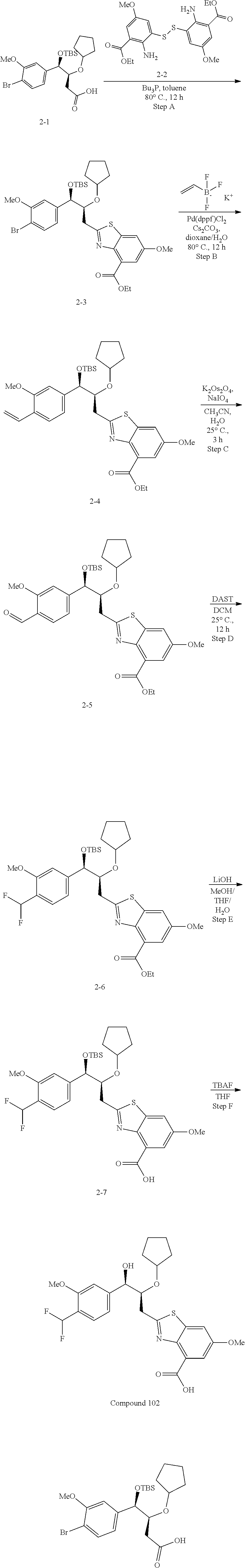

(3S,4R)-4-(4-bromo-3-methoxyphenyl)-4-((tert-butyldimethylsilyl)oxy)-3-(cyclopentyloxy)butanoic acid (2-1) was synthesized according to the procedures described for the preparation of Example A1 (step H to step P in Scheme 1) by using 4-bromo-3-methoxy-benzaldehyde in step H.

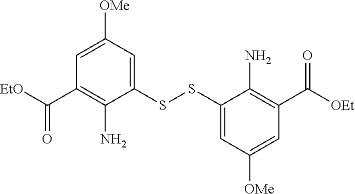 [SMILES: CCOC(=O)c1cc(OC)cc(SSc2cc(OC)cc(C(=O)OCC)c2N)c1N]

Ethyl 2-amino-3-[(2-amino-3-ethoxycarbonyl-5-methoxy-phenyl)disulfanyl]-5-methoxy-benzoate (2-2) was synthesized according to the procedures described for the preparation of Example A1 (step C to step D in Scheme 1) by using methyl 2-amino-3-bromo-5-methoxybenzoate in step C. ¹H NMR (400 MHz, CDCl3) δ 7.53 (d, J=3.2 Hz, 2H), 6.88 (d, J=3.2 Hz, 2H), 4.35 (q, J=7.2 Hz, 4H), 3.63 (s, 6H), 1.40 (t, J=7.2 Hz, 6H).

### Step A: ethyl 2-((2S,3R)-3-(4-bromo-3-methoxyphenyl)-3-((tert-butyldimethylsilyl)oxy)-2-(cyclopentyloxy)propyl)-6-methoxybenzo[d]thiazole-4-carboxylate

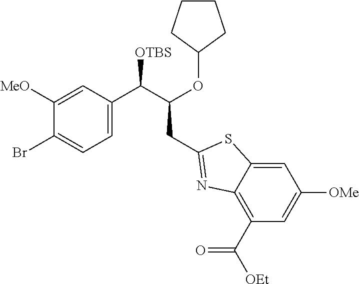 [SMILES: CCOC(=O)c1cc(OC)cc2sc(C[C@H](OC3CCCC3)[C@H](O[Si](C)(C)C(C)(C)C)c3ccc(Br)c(OC)c3)nc12]

To a solution of (3S,4R)-4-(4-bromo-3-methoxyphenyl)-4-((tert-butyldimethylsilyl)oxy)-3-(cyclopentyloxy)butanoic acid (850 mg, 1.74 mmol) and ethyl 2-amino-3-[(2-amino-3-ethoxycarbonyl-phenyl) disulfanyl]benzoate (789 mg, 1.74 mmol) in toluene (10 mL) was added tributylphosphane (1.06 g, 5.23 mmol). The reaction mixture was stirred at 80° C. for 12 h. After cooling, the mixture was quenched with H₂O (30 mL), extracted with EtOAc (30 mL×2). The combined organic layers were washed with brine (50 mL), dried over anhydrous Na₂SO₄, filtered and concentrated. The residue was purified by silica gel column (PE/EtOAc=10/1) to afford ethyl 2-[(2S,3R)-3-(4-bromo-3-methoxy-phenyl)-3-[tert-butyl(dimethyl)silyl]oxy-2-(cyclopentoxy)propyl]-6-methoxy-1,3-benzothiazole-4-carboxylate (500 mg, 42.2% yield) as yellow oil. LC-MS: m/z 680.1 (M+H)⁺.

### Step B: ethyl 2-((2S,3R)-3-((tert-butyldimethylsilyl)oxy)-2-(cyclopentyloxy)-3-(3-methoxy-4-vinylphenyl)propyl)-6-methoxybenzo[d]thiazole-4-carboxylate

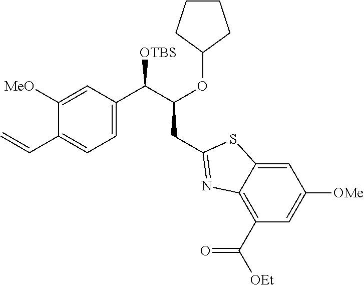 [SMILES: C=Cc1ccc([C@@H](O[Si](C)(C)C(C)(C)C)[C@H](Cc2nc3c(C(=O)OCC)cc(OC)cc3s2)OC2CCCC2)cc1OC]

To a solution of ethyl 2-[(2S,3R)-3-(4-bromo-3-methoxy-phenyl)-3-[tert-butyl(dimethyl)silyl]oxy-2-(cyclopentoxy)propyl]-6-methoxy-1,3-benzothiazole-4-carboxylate (330 mg, 486.19 μmol) in H₂O (2 mL) and dioxane (8 mL) was added Pd(dppf)Cl₂ (36 mg, 48.62 μmol), potassium ethenyltrifluoroborate (130 mg, 972.38 μmol) and Cs₂CO₃ (475 mg, 1.46 mmol). The mixture was stirred at 80° C. for 12 h. After cooling, the mixture was diluted with H₂O (30 mL), extracted with EtOAc (30 mL×2). The combined organic layers were washed with brine (50 mL), dried over anhydrous Na₂SO₄, filtered and concentrated. The residue was purified by silica gel column (PE/EtOAc=10/1) to afford ethyl 2-[(2S,3R)-3-[tert-butyl(dimethyl)silyl]oxy-2-(cyclopentoxy)-3-(3-methoxy-4-vinyl-phenyl)propyl]-6-methoxy-1,3-benzothiazole-4-carboxylate (240 mg, 78.8% yield). LC-MS: m/z 626.2 (M+H)⁺.

### Step C: ethyl 2-((2S,3R)-3-((tert-butyldimethylsilyl)oxy)-2-(cyclopentyloxy)-3-(4-formyl-3-methoxyphenyl)propyl)-6-methoxybenzo[d]thiazole-4-carboxylate

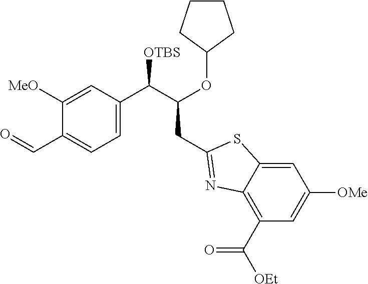 [SMILES: CCOC(=O)c1cc(OC)cc2sc(C[C@H](OC3CCCC3)[C@H](O[Si](C)(C)C(C)(C)C)c3ccc(C=O)c(OC)c3)nc12]

To a solution of ethyl 2-[(2S,3R)-3-[tert-butyl(dimethyl)silyl]oxy-2-(cyclopentoxy)-3-(3-methoxy-4-vinyl-phenyl)propyl]-6-methoxy-1,3-benzothiazole-4-carboxylate (200 mg, 319.55 μmol) in THF (3 mL) and H₂O (3 mL) was added NaIO₄ (273 mg, 1.28 mmol) and K₂OsO₄·2H₂O (1 mg, 1.92 μmol). The reaction mixture was stirred at 25° C. for 3 h. The mixture was quenched with H₂O (30 mL), extracted with EtOAc (30 mL×2). The combined organic layers were washed with brine (50 mL), dried over anhydrous Na₂SO₄, filtered and concentrated. The residue was purified by silica gel column (PE/EtOAc=10/1) to afford ethyl 2-[(2S,3R)-3-[tert-butyl(dimethyl)silyl]oxy-2-(cyclopentoxy)-3-(4-formyl-3-methoxy-phenyl)propyl]-6-methoxy-1,3-benzothiazole-4-carboxylate (150 mg, 74.7% yield). LC-MS: m/z 628.3 (M+H)⁺.

### Step D: ethyl 2-((2S,3R)-3-((tert-butyldimethylsilyl)oxy)-2-(cyclopentyloxy)-3-(4-(difluoromethyl)-3-methoxyphenyl)propyl)-6-methoxybenzo[d]thiazole-4-carboxylate

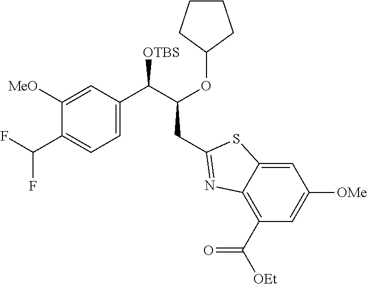 [SMILES: CCOC(=O)c1cc(OC)cc2sc(C[C@H](OC3CCCC3)[C@H](O[Si](C)(C)C(C)(C)C)c3ccc(C(F)F)c(OC)c3)nc12]

To a solution of ethyl 2-[(2S,3R)-3-[tert-butyl(dimethyl)silyl]oxy-2-(cyclopentoxy)-3-(4-formyl-3-methoxy-phenyl)propyl]-6-methoxy-1,3-benzothiazole-4-carboxylate (150 mg, 238.91 μmol) in DCM (3 mL) was added DAST (192.55 mg, 1.19 mmol) at 0° C. The mixture was stirred at 25° C. for 12 h. The mixture was quenched with sat. aq. NaHCO₃ (30 mL), extracted with DCM (30 mL×2). The combined organic layers were washed with brine (50 mL), dried over anhydrous Na₂SO₄, filtered and concentrated. The residue was purified by silica gel column (PE/EtOAc=10/1) to afford ethyl 2-[(2S,3R)-3-[tert-butyl(dimethyl)silyl]oxy-2-(cyclopentoxy)-3-[4-(difluoromethyl)-3-methoxy-phenyl]propyl]-6-methoxy-1,3-benzothiazole-4-carboxylate (70 mg, 45.1% yield). LC-MS: m/z 672.2 (M+Na)⁺.

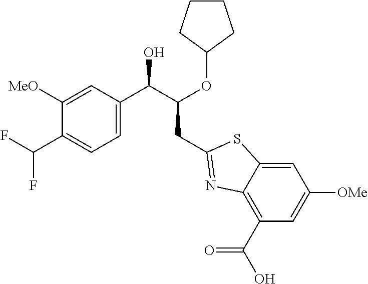 [SMILES: COc1cc(C(=O)O)c2nc(C[C@H](OC3CCCC3)[C@H](O)c3ccc(C(F)F)c(OC)c3)sc2c1]

2-((2S,3R)-2-(cyclopentyloxy)-3-(4-(difluoromethyl)-3-methoxyphenyl)-3-hydroxypropyl)-6-methoxybenzo[d]thiazole-4-carboxylic acid (compound 102) was synthesized according to the procedures described for the preparation of Example A1 (step R to step S in Scheme 1) by using ethyl 2-((2S,3R)-3-((tert-butyldimethylsilyl)oxy)-2-(cyclopentyloxy)-3-(4-(difluoromethyl)-3-methoxyphenyl)propyl)-6-methoxybenzo[d]thiazole-4-carboxylate in step R. LC-MS: m/z 508.0 (M+H)⁺. ¹H NMR (400 MHz, CD₃OD) δ 7.75-7.72 (m, 1H), 7.71-7.68 (m, 1H), 7.46 (d, J=8.0 Hz, 1H), 7.16 (s, 1H), 7.09 (d, J=7.8 Hz, 1H), 7.06-6.76 (m, 1H), 6.92 (t, J=55.6 Hz, 11H), 4.74 (d, J=4.0 Hz, 1H), 3.99-3.93 (m, 2H), 3.91 (s, 3H), 3.90 (s, 3H), 3.52-3.39 (m, 2H), 1.58-1.45 (m, 3H), 1.42-1.30 (m, 5H).

### Example A3

### 2-((2S,3R)-2-(cyclopentyloxy)-3-(3,5-dimethoxy-4-methylphenyl)-3-hydroxypropyl)-6-(hydroxymethyl)benzo[d]thiazole-4-carboxylic acid (Compound 103)

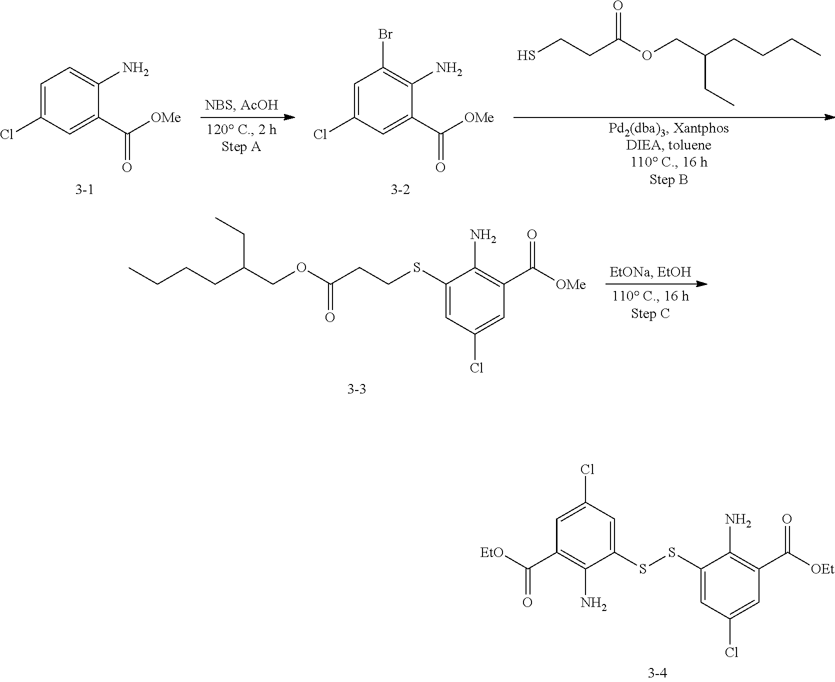

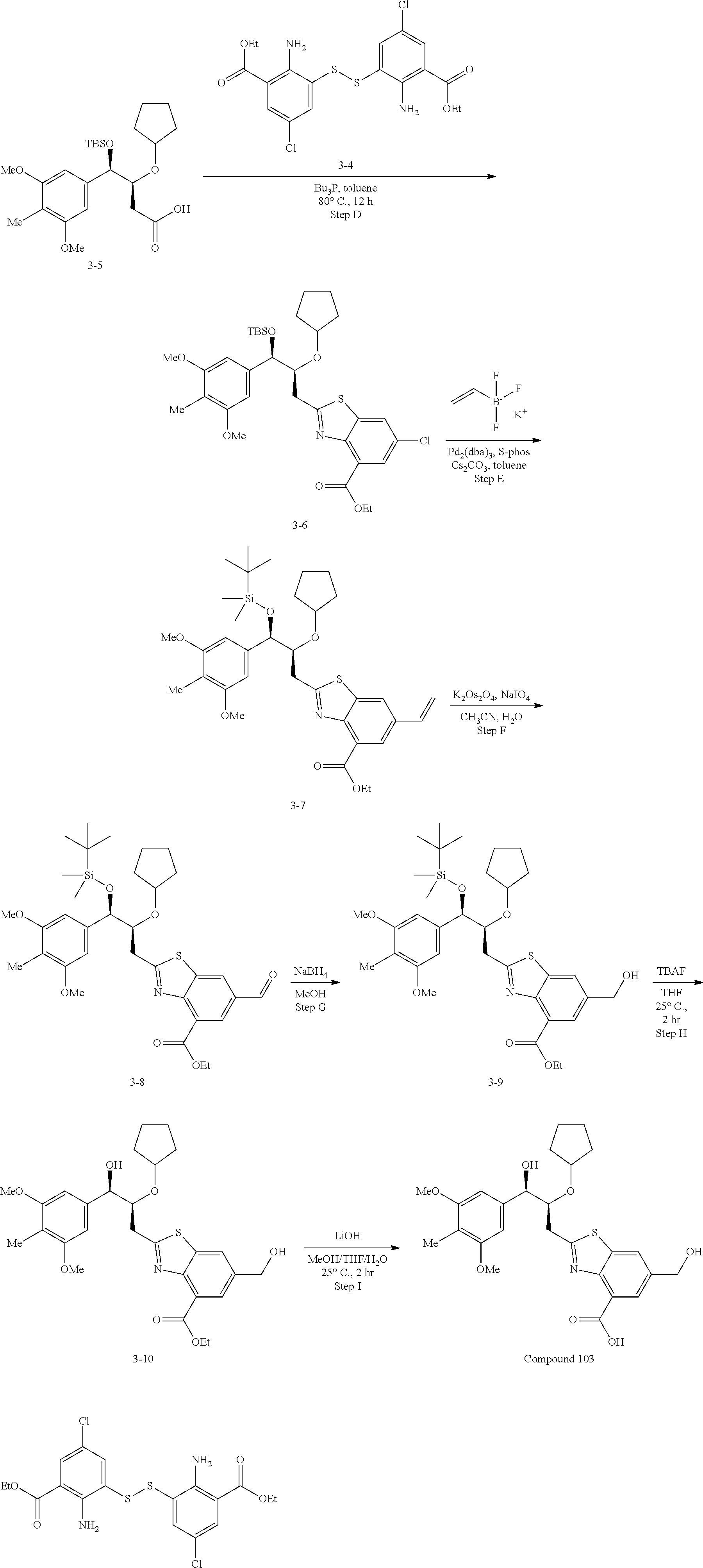

Diethyl 3,3′-disulfanediylbis(2-amino-5-chlorobenzoate) (3-4) was synthesized according to the procedures described for the preparation of Example A1 (step B to step D in Scheme 1) by using methyl 2-amino-5-chloro-benzoate in step B. LC-MS: m/z 460.9 (M+H)⁺.

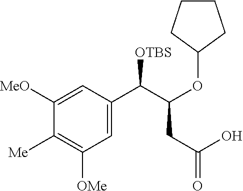 [SMILES: COc1cc([C@@H](O[Si](C)(C)C(C)(C)C)[C@H](CC(=O)O)OC2CCCC2)cc(OC)c1C]

(3S,4R)-4-((tert-butyldimethylsilyl)oxy)-3-(cyclopentyloxy)-4-(3,5-dimethoxy-4-methylphenyl)butanoic acid (3-5) was synthesized according to the procedures described for the preparation of Example A1 (step H to step P in Scheme 1) by using 3,5-dimethoxy-4-methylbenzene-1-carbaldehyde in step H. ¹H NMR (400 MHz, CDCl₃) b 6.51 (s, 2H), 4.71-4.70 (m, 1H), 3.98-3.95 (m, 1H), 3.84-3.77 (m, 7H), 2.60-2.57 (m, 2H), 2.06 (s, 3H), 1.67-1.26 (m, 8H), 0.91 (s, 9H), 0.07 (s, 3H), −0.11 (s, 3H).

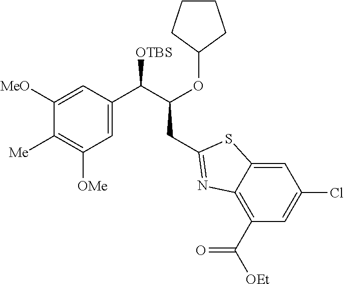 [SMILES: CCOC(=O)c1cc(Cl)cc2sc(C[C@H](OC3CCCC3)[C@H](O[Si](C)(C)C(C)(C)C)c3cc(OC)c(C)c(OC)c3)nc12]

Ethyl 2-((2S,3R)-3-((tert-butyldimethylsilyl)oxy)-2-(cyclopentyloxy)-3-(3,5-dimethoxy-4-methylphenyl)propyl)-6-chlorobenzo[d]thiazole-4-carboxylate (3-6) was synthesized according to the procedures described for the preparation of Example A1 (step Q in Scheme 1) by diethyl 3,3′-disulfanediylbis(2-amino-5-chlorobenzoate) (3-4) and (3S,4R)-4-((tert-butyldimethylsilyl)oxy)-3-(cyclopentyloxy)-4-(3,5-dimethoxy-4-methylphenyl)butanoic acid (3-5) in step Q. ¹H NMR (400 MHz, CDCl₃) δ 7.96-7.99 (m, 2H), 6.55 (s, 2H), 4.75-4.74 (m, 1H), 4.49-4.44 (m, 2H), 3.94-3.92 (m, 1H), 3.83-3.82 (m, 7H), 3.46-3.35 (m, 2H), 2.05 (s, 3H), 1.51-1.32 (m, 11H), 0.91 (s, 9H), 0.07 (s, 3H), −0.12 (s, 3H).

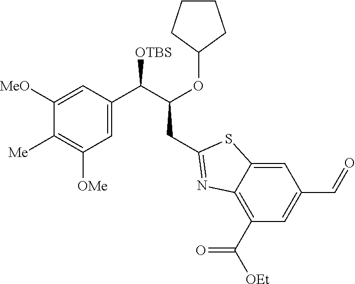 [SMILES: CCOC(=O)c1cc(C=O)cc2sc(C[C@H](OC3CCCC3)[C@H](O[Si](C)(C)C(C)(C)C)c3cc(OC)c(C)c(OC)c3)nc12]

Ethyl 2-((2S,3R)-3-((tert-butyldimethylsilyl)oxy)-2-(cyclopentyloxy)-3-(3,5-dimethoxy-4-methylphenyl)propyl)-6-formylbenzo[d]thiazole-4-carboxylate (3-8) was synthesized according to the procedures described for the preparation of Example A2 (step B to step C in Scheme 2) by using ethyl 2-((2S,3R)-3-((tert-butyldimethylsilyl)oxy)-2-(cyclopentyloxy)-3-(3,5-dimethoxy-4-methylphenyl)propyl)-6-chlorobenzo[d]thiazole-4-carboxylate (3-6). LC-MS: m/z 642.2 (M+H)⁺.

### Step G: ethyl 2-((2S,3R)-3-((tert-butyldimethylsilyl)oxy)-2-(cyclopentyloxy)-3-(3,5-dimethoxy-4-methylphenyl)propyl)-6-(hydroxymethyl)benzo[d]thiazole-4-carboxylate

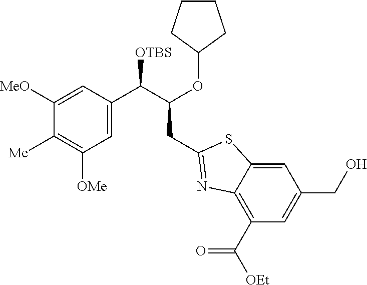 [SMILES: CCOC(=O)c1cc(CO)cc2sc(C[C@H](OC3CCCC3)[C@H](O[Si](C)(C)C(C)(C)C)c3cc(OC)c(C)c(OC)c3)nc12]

To a solution of ethyl 2-((2S,3R)-3-((tert-butyldimethylsilyl)oxy)-2-(cyclopentyloxy)-3-(3,5-dimethoxy-4-methylphenyl)propyl)-6-formylbenzo[d] thiazole-4-carboxylate (30 mg, 46.78 μmol) in MeOH (1 mL) was added NaBH₄ (5.30 mg, 140.21 μmol). The reaction mixture was stirred at 25° C. for 3 h. Then the mixture was quenched with sat.aq. NH₄Cl solution (10 mL), extracted with DCM (10 mL×3). The combined organic layers were washed with brine (10 mL, dried over anhydrous Na₂SO₄, filtered and concentrated to give ethyl 2-((2S,3R)-3-((tert-butyldimethylsilyl)oxy)-2-(cyclopentyloxy)-3-(3,5-dimethoxy-4-methylphenyl) propyl)-6-(hydroxymethyl)benzo[d]thiazole-4-carboxylate (20 mg, 66% yield). LC-MS: m/z 644.3 (M+H)⁺.

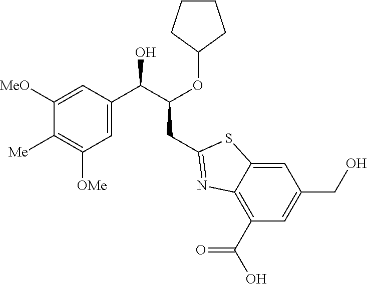 [SMILES: COc1cc([C@@H](O)[C@H](Cc2nc3c(C(=O)O)cc(CO)cc3s2)OC2CCCC2)cc(OC)c1C]

2-((2S,3R)-2-(cyclopentyloxy)-3-(3,5-dimethoxy-4-methylphenyl)-3-hydroxypropyl)-6-(hydroxymethyl)benzo[d]thiazole-4-carboxylic acid (Compound 103) was synthesized according to the procedures described for the preparation of Example A1 (step S and step R in Scheme 1) by using ethyl 2-((2S,3R)-3-((tert-butyldimethylsilyl)oxy)-2-(cyclopentyloxy)-3-(3,5-dimethoxy-4-methylphenyl) propyl)-6-(hydroxymethyl)benzo[d]thiazole-4-carboxylate (3-9) in step S. LC-MS: m/z 502.3 (M+H)⁺. ¹H NMR (400 MHz, CD₃OD) δ 8.05 (s, 1H), 7.94 (s, 1H), 6.64 (s, 2H), 4.75 (s, 1H), 4.61 (s, 2H), 3.97-3.93 (m, 1H), 3.86-3.79 (m, 1H), 3.79 (s, 6H), 3.45-3.43 (m, 2H), 3.30-3.29 (m, 1H), 1.99 (s, 3H), 1.42-1.32 (m, 8H).

### Example A4

### 2-((2S,3R)-2-(cyclopentyloxy)-3-(3,5-dimethoxy-4-methylphenyl)-3-hydroxypropyl)-6-(methoxymethyl)benzo[d]thiazole-4-carboxylic acid (Compound 104)

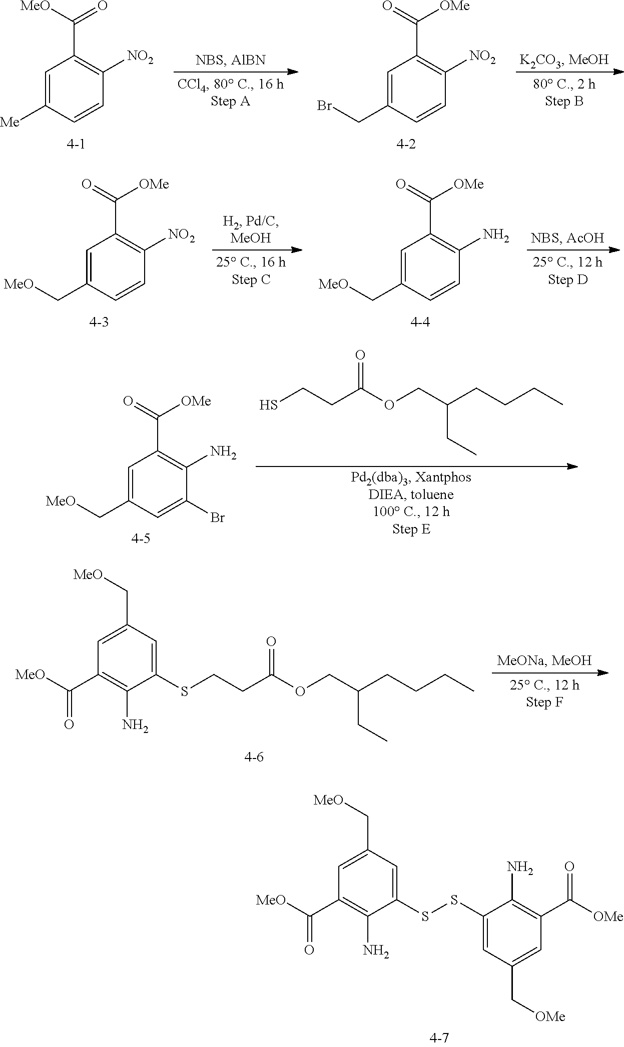

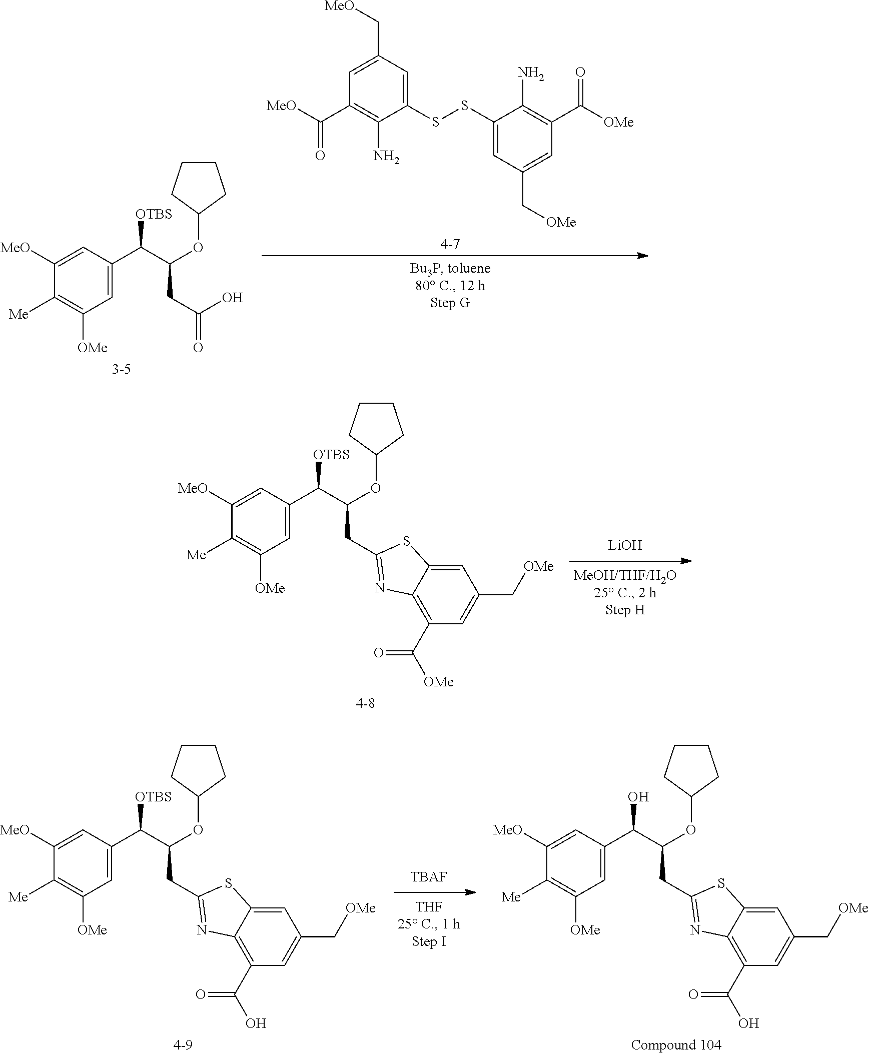

### Step A: methyl 5-(bromomethyl)-2-nitrobenzoate

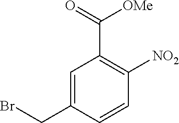 [SMILES: COC(=O)c1cc(CBr)ccc1[N+](=O)[O-]]

To a solution of methyl 5-methyl-2-nitro-benzoate (23.5 g, 120.41 mmol) in CCl₄ (200 mL) was added AIBN (1.98 g, 12.04 mmol) and NBS (32.15 g, 180.61 mmol). The reaction mixture was stirred at 80° C. for 16 h. After cooling, the reaction was diluted with H₂O (300 mL), extracted with DCM (300 mL×3). The combined organics were concentrated under reduced pressure to give a residue. The residue was purified by column (PE/EtOAc=7/1) to give methyl 5-(bromomethyl)-2-nitrobenzoate (19.12 g, 38.6% yield).

### Step B: methyl 5-(methoxymethyl)-2-nitrobenzoate

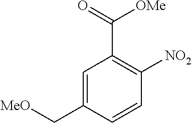 [SMILES: COCc1ccc([N+](=O)[O-])c(C(=O)OC)c1]

To a solution of methyl 5-(bromomethyl)-2-nitrobenzoate (19.12 g, 69.77 mmol) in MeOH (180 mL) was added K₂CO₃ (9.64 g, 69.77 mmol). The mixture was stirred at 80° C. for 2 h. The reaction was diluted with H₂O (300 mL), extracted with EtOAc (300 mL×3). The organics were dried over anhydrous Na₂SO₄, filtered and concentrated under reduced pressure to give a residue. The residue was purified by silica gel column (PE/EtOAc=5/1) to give the methyl 5-(methoxymethyl)-2-nitrobenzoate (10.52 g, 66.9% yield). ¹H NMR (400 MHz, CDCl₃) δ 7.85 (d, J=8.4 Hz, 1H), 7.60 (d, J=1.2 Hz, 1H), 7.51 (d, J=8.4 Hz, 1H), 4.48 (s, 2H), 3.86 (s, 3H), 3.38 (s, 3H).

### Step C: methyl 2-amino-5-(methoxymethyl)benzoate

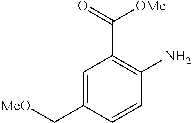 [SMILES: COCc1ccc(N)c(C(=O)OC)c1]

To a solution of methyl 5-(methoxymethyl)-2-nitro-benzoate (7.52 g, 33.39 mmol) in EtOAc (100 mL) was added 10% Pd/C (2.6 g) under N₂ and the mixture was degassed and purged with N₂ for 3 times. Then the mixture was degassed and purged with H₂ for 3 times. The mixture was stirred at 25° C. under H₂ (45 psi) for 16 h. After filtration, the filtrate was concentrated under reduced pressure to give a residue. The residue was purified by silica gel column (PE/EtOAc=6/1) to give the methyl 2-amino-5-(methoxymethyl)benzoate (5.85 g, 79.6% yield). LC-MS: m/z 195.8 (M+H)⁺.

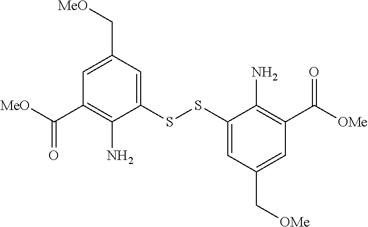 [SMILES: COCc1cc(SSc2cc(COC)cc(C(=O)OC)c2N)c(N)c(C(=O)OC)c1]

Dimethyl 3,3′-disulfanediylbis(2-amino-5-(methoxymethyl)benzoate) (4-7) was synthesized according to the procedures described for the preparation of Example A1 (step B to step D in Scheme 1) by using methyl 2-amino-5-(methoxymethyl)benzoate in step B. LC-MS: m/z 474.8 (M+Na)⁺.

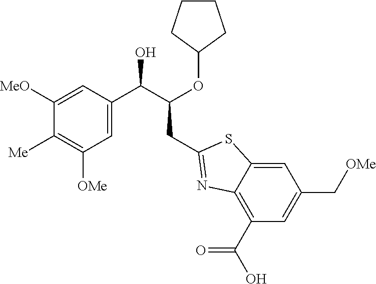 [SMILES: COCc1cc(C(=O)O)c2nc(C[C@H](OC3CCCC3)[C@H](O)c3cc(OC)c(C)c(OC)c3)sc2c1]

2-((2S,3R)-2-(cyclopentyloxy)-3-(3,5-dimethoxy-4-methylphenyl)-3-hydroxypropyl)-6-(methoxymethyl)benzo[d]thiazole-4-carboxylic acid (Compound 104) was synthesized according to the procedures described for the preparation of Example A1 (step Q to step S in Scheme 1) by using dimethyl 3,3′-disulfanediylbis(2-amino-5-(methoxymethyl)benzoate) (4-7) and (3S,4R)-4-((tert-butyldimethylsilyl)oxy)-3-(cyclopentyloxy)-4-(3,5-dimethoxy-4-methylphenyl)butanoic acid (3-5) in step Q. LC-MS: m/z 516.1 (M+H)⁺. ¹H NMR (400 MHz, CD₃OD) δ 8.19 (d, J=1.2 Hz, 1H), 8.12 (d, J=1.6 Hz, 1H), 6.67 (s, 2H), 4.69 (d, J=5.6 Hz, 1H), 4.65 (s, 2H), 4.04-3.99 (m, 1H), 3.97-3.90 (m, 1H), 3.83 (s, 6H), 3.53-3.48 (m, 2H), 3.46 (s, 3H), 1.99 (s, 3H), 1.55-1.34 (m, 8H).

### Example A5

### 2-((2S,3R)-2-(cyclopentyloxy)-3-(3,5-dimethoxy-4-methylphenyl)-3-hydroxypropyl)-6-(methylamino)benzo[d]thiazole-4-carboxylic acid (Compound 105)

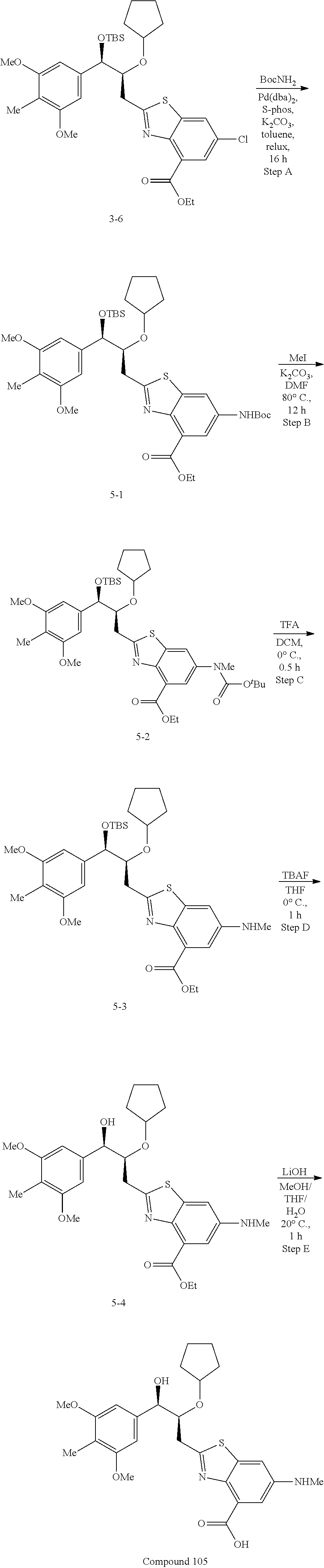

### Step A: Ethyl 6-((tert-butoxycarbonyl)amino)-2-((2S,3R)-3-((tert-butyldimethylsilyl)oxy)-2-(cyclopentyloxy)-3-(3,5-dimethoxy-4-methylphenyl)propyl)benzo[d]thiazole-4-carboxylate

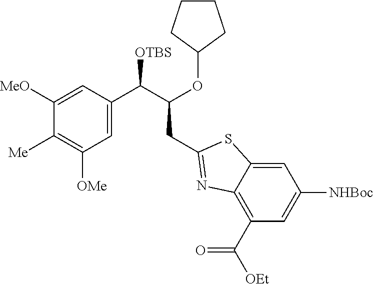 [SMILES: CCOC(=O)c1cc(NC(=O)OC(C)(C)C)cc2sc(C[C@H](OC3CCCC3)[C@H](O[Si](C)(C)C(C)(C)C)c3cc(OC)c(C)c(OC)c3)nc12]

To a solution of ethyl 2-[(2S,3R)-3-[tert-butyl (dimethyl) silyl]oxy-2-(cyclopentoxy)-3-(3,5-dimethoxy-4-methyl-phenyl) propyl]-6-chloro-1,3-benzothiazole-4-carboxylate (500 mg, 771.22 μmol) and tert-butyl carbamate (271.04 mg, 2.31 mmol) in toluene (10 mL) was added Pd(dba)₂ (44.35 mg, 77.12 μmol), K₂CO₃ (319.76 mg, 2.31 mmol) and S-Phos (63.32 mg, 154.24 μmol). The mixture was stirred at 100° C. for 16 h. The reaction mixture was quenched by addition of H₂O (10 mL) at 0° C., extracted with EtOAc (10 mL×3). The combined organic layers were washed with brine (10 mL), dried over anhydrous Na₂SO₄, filtered and concentrated under reduced pressure to give a residue. The residue was purified by silica gel column (PE/EtOAc=100/1 to 10/1) to afford ethyl 6-(tert-butoxycarbonylamino)-2-[(2S,3R)-3-[tert-butyl (dimethyl) silyl]oxy-2-(cyclopentoxy)-3-(3,5-dimethoxy-4-methyl-phenyl) propyl]-1,3-benzothiazole-4-carboxylate (500 mg, 88.9% yield). ¹H NMR (400 MHz, CDCl₃) δ 8.46 (br s, 1H), 7.66 (d, J=2.1 Hz, 1H), 6.70 (s, 1H), 6.56 (s, 2H), 4.73 (d, J=4.6 Hz, 1H), 4.48-4.42 (m, 3H), 3.95-3.90 (m, 1H), 3.82 (s, 6H), 3.45-3.40 (m, 1H), 3.31-3.25 (m, 1H), 2.06 (s, 3H), 1.53 (s, 9H), 1.43-1.25 (m, 10H), 1.43-1.24 (m, 1H), 0.91 (s, 9H), 0.07 (s, 3H), −0.12 (s, 3H).

### Step B Ethyl 6-((tert-butoxycarbonyl)(methyl)amino)-2-((2S,3R)-3-((tert-butyldimethylsilyl)oxy)-2-(cyclopentyloxy)-3-(3,5-dimethoxy-4-methylphenyl)propyl)benzo[d]thiazole-4-carboxylate

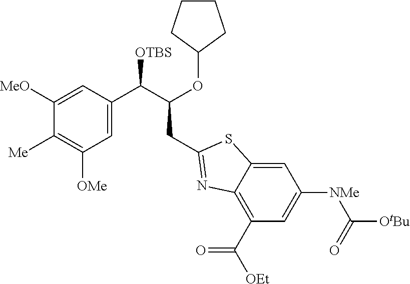 [SMILES: CCOC(=O)c1cc(N(C)C(=O)OC(C)(C)C)cc2sc(C[C@H](OC3CCCC3)[C@H](O[Si](C)(C)C(C)(C)C)c3cc(OC)c(C)c(OC)c3)nc12]

To a solution of ethyl 6-(tert-butoxycarbonylamino)-2-[(2S,3R)-3-[tert-butyl (dimethyl) silyl]oxy-2-(cyclopentoxy)-3-(3,5-dimethoxy-4-methyl-phenyl) propyl]-1,3-benzothiazole-4-carboxylate (200 mg, 274.35 μmol) in DMF (2 mL) was added K₂CO₃ (189.58 mg, 1.37 mmol) and MeI (194.70 mg, 85.40 μL). The mixture was stirred at 80° C. for 12 h. After cooling, the reaction mixture was diluted with H₂O (5 mL), extracted with EtOAc (5 mL×3). The combined organic layers were washed with brine (5 mL×3), dried over anhydrous Na₂SO₄, filtered and concentrated under reduced pressure to give a residue. The residue was purified by prep. TLC (SiO₂, PE/EtOAc=10/1) to afford ethyl 6-[tert-butoxycarbonyl (methyl) amino]-2-[(2S,3R)-3-[tert-butyl (dimethyl) silyl]oxy-2-(cyclopentoxy)-3-(3,5-dimethoxy-4-methyl-phenyl) propyl]-1,3-benzothiazole-4-carboxylate (40 mg, 19.6% yield). LC-MS: m/z 765.3 (M+Na)⁺.

### Step C: Ethyl 2-((2S,3R)-3-((tert-butyldimethylsilyl)oxy)-2-(cyclopentyloxy)-3-(3,5-dimethoxy-4-methylphenyl)propyl)-6-(methylamino)benzo[d]thiazole-4-carboxylate

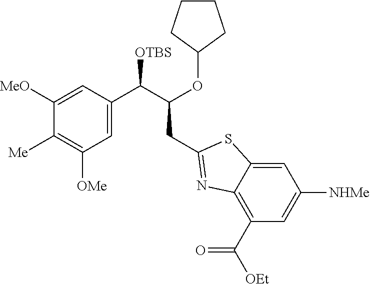 [SMILES: CCOC(=O)c1cc(NC)cc2sc(C[C@H](OC3CCCC3)[C@H](O[Si](C)(C)C(C)(C)C)c3cc(OC)c(C)c(OC)c3)nc12]

To a solution of ethyl 6-[tert-butoxycarbonyl (methyl) amino]-2-[(2S,3R)-3-[tert-butyl (dimethyl) silyl]oxy-2-(cyclopentoxy)-3-(3,5-dimethoxy-4-methyl-phenyl) propyl]-1,3-benzothiazole-4-carboxylate (30 mg, 40.37 μmol) in DCM (1 mL) was added TFA (29.89 μL, 400 μmol). The reaction mixture was stirred at 0° C. for 0.5 h. The reaction mixture was diluted with H₂O (10 mL), extracted with EtOAc (10 mL×3). The combined organic layers were washed with brine (10 mL), dried over anhydrous Na₂SO₄, filtered and concentrated to afford ethyl 2-((2S,3R)-3-((tert-butyldimethylsilyl) oxy)-2-(cyclopentyloxy)-3-(3,5-dimethoxy-4-methylphenyl) propyl)-6-(methylamino) benzo[d]thiazole-4-carboxylate (22.5 mg, 86.7% yield), which was used for next step without further purification. LC-MS: m/z 643.1 (M+H)⁺.

### Step D: Ethyl 2-((2S,3R)-2-(cyclopentyloxy)-3-(3,5-dimethoxy-4-methylphenyl)-3-hydroxypropyl)-6-(methylamino)benzo[d]thiazole-4-carboxylate

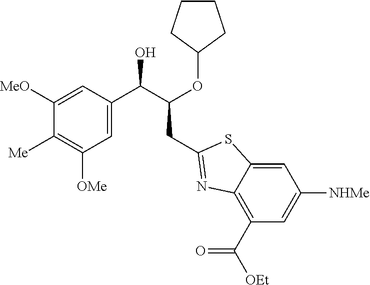 [SMILES: CCOC(=O)c1cc(NC)cc2sc(C[C@H](OC3CCCC3)[C@H](O)c3cc(OC)c(C)c(OC)c3)nc12]

To a solution of ethyl 2-[(2S,3R)-3-[tert-butyl (dimethyl) silyl]oxy-2-(cyclopentoxy)-3-(3,5-dimethoxy-4-methyl-phenyl) propyl]-6-(methylamino)-1,3-benzothiazole-4-carboxylate (30 mg, 46.66 μmol) in THF (0.5 mL) was added TBAF (466.62 μL, 466.62 μmol, 1 M solution in THF) at 0° C. The mixture was stirred at 0° C. for 1 h. The reaction mixture was diluted with H₂O (10 mL) and extracted with EtOAc (10 mL×3). The combined organic layers were washed with brine (20 mL), dried over anhydrous Na₂SO₄, filtered and concentrated to afford ethyl 2-[(2S,3R)-2-(cyclopentoxy)-3-(3,5-dimethoxy-4-methyl-phenyl)-3-hydroxy-propyl]-6-(methylamino)-1,3-benzothiazole-4-carboxylate (20 mg, 81.1% yield), which used for next step without further purification. LC-MS: m/z 528.7 (M+H)⁺.

### Step E: 2-((2S,3R)-2-(cyclopentyloxy)-3-(3,5-dimethoxy-4-methylphenyl)-3-hydroxypropyl)-6-(methylamino)benzo[d]thiazole-4-carboxylic acid (Compound 105)

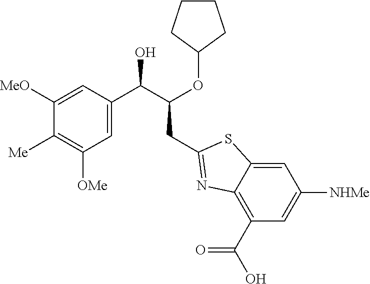 [SMILES: CNc1cc(C(=O)O)c2nc(C[C@H](OC3CCCC3)[C@H](O)c3cc(OC)c(C)c(OC)c3)sc2c1]

To a solution of ethyl 2-[(2S,3R)-2-(cyclopentoxy)-3-(3,5-dimethoxy-4-methyl-phenyl)-3-hydroxy-propyl]-6-(methylamino)-1,3-benzothiazole-4-carboxylate (20 mg, 37.83 μmol) in THF (0.8 mL), MeOH (0.2 mL) and H₂O (0.2 mL) was added LiOH (4.53 mg, 189.16 μmol). The mixture was stirred at 20° C. for 1 h. The reaction mixture was diluted with H₂O (10 mL), extracted with EtOAc (10 mL×3). The combined organic layers were washed with brine (10 mL), dried over anhydrous Na₂SO₄, filtered and concentrated. The residue was purified by prep. HPLC (Column: Kromasil 100-5-C18; Mobile Phase A: Water (0.1% HCOOH), Mobile Phase B: CH₃CN; Gradient: 50% B to 90% B in 10 min) to give 2-[(2S,3R)-2-(cyclopentoxy)-3-(3,5-dimethoxy-4-methyl-phenyl)-3-hydroxy-propyl]-6-(methylamino)-1,3-benzothiazole-4-carboxylic acid (7.92 mg, 41.8% yield). LC-MS: m/z 501.3 (M+H)⁺. ¹H NMR (400 MHz, CDCl₃) δ 7.57 (d, J=2.3 Hz, 1H), 7.09 (d, J=2.3 Hz, 1H), 6.57 (s, 2H), 4.93 (d, J=4.3 Hz, 1H), 4.02-3.97 (m, 2H), 3.84 (s, 6H), 3.31-3.24 (m, 1H), 3.11 (dd, J=3.5, 15.2 Hz, 1H), 2.92 (s, 3H), 2.06 (s, 3H), 1.61-1.40 (m, 8H).

### Example A6

### 2-((2S,3R)-2-(cyclopentyloxy)-3-(3,5-dimethoxy-4-methylphenyl)-3-hydroxypropyl)-6-(dimethylamino)benzo[d]thiazole-4-carboxylic acid (Compound 106)

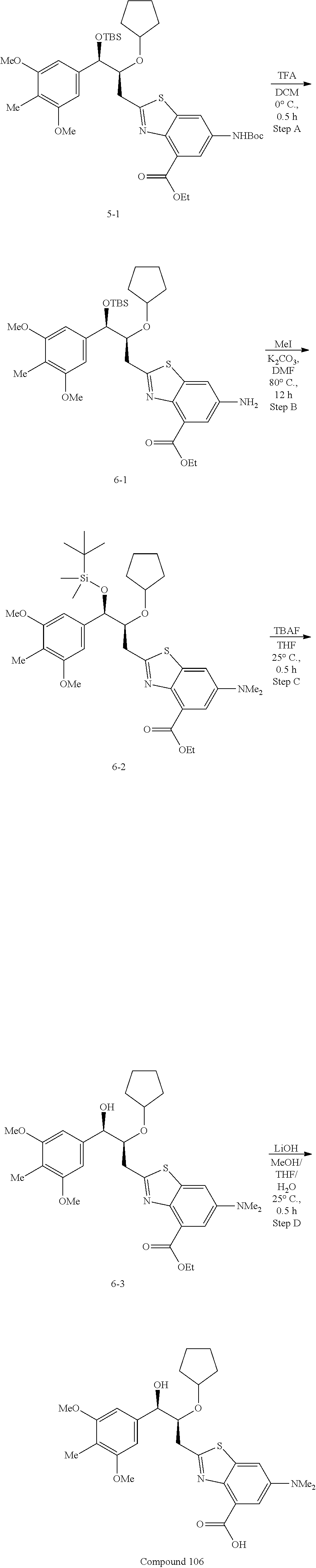

### Step A: ethyl 6-amino-2-((2S,3R)-3-((tert-butyldimethylsilyl)oxy)-2-(cyclopentyloxy)-3-(3,5-dimethoxy-4-methylphenyl)propyl)benzo[d]thiazole-4-carboxylate

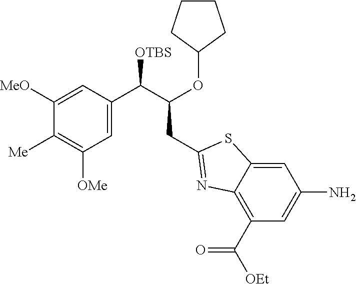 [SMILES: CCOC(=O)c1cc(N)cc2sc(C[C@H](OC3CCCC3)[C@H](O[Si](C)(C)C(C)(C)C)c3cc(OC)c(C)c(OC)c3)nc12]

To a solution of ethyl 6-(tert-butoxycarbonylamino)-2-[(2S,3R)-3-[tert-butyl (dimethyl) silyl]oxy-2-(cyclopentoxy)-3-(3,5-dimethoxy-4-methyl-phenyl) propyl]-1,3-benzothiazole-4-carboxylate (100 mg, 137.17 μmol) in DCM (1 mL) was added TFA (101.56 μL, 1.37 mmol). The reaction mixture was stirred at 0° C. for 0.5 h. The reaction mixture was quenched by addition H₂O (10 mL), extracted with DCM (10 mL×3). The organic layers were dried over Na₂SO₄, filtered and concentrated under reduced pressure to afford ethyl 6-amino-2-[(2S,3R)-3-[tert-butyl (dimethyl) silyl]oxy-2-(cyclopentoxy)-3-(3,5-dimethoxy-4-methyl-phenyl) propyl]-1,3-benzothiazole-4-carboxylate (80 mg, 92.74% yield), which used for next step without further purification. LC-MS: m/z 629.2 (M+H)⁺.

### Step B: ethyl 2-((2S,3R)-3-((tert-butyldimethylsilyl)oxy)-2-(cyclopentyloxy)-3-(3,5-dimethoxy-4-methylphenyl)propyl)-6-(dimethylamino)benzo[d]thiazole-4-carboxylate

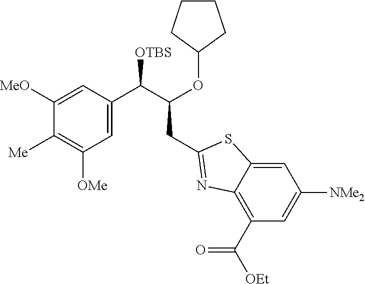 [SMILES: CCOC(=O)c1cc(N(C)C)cc2sc(C[C@H](OC3CCCC3)[C@H](O[Si](C)(C)C(C)(C)C)c3cc(OC)c(C)c(OC)c3)nc12]

To a solution of ethyl 6-amino-2-((2S,3R)-3-((tert-butyldimethylsilyl) oxy)-2-(cyclopentyloxy)-3-(3,5-dimethoxy-4-methylphenyl) propyl) benzo[d]thiazole-4-carboxylate (60 mg, 95.41 μmol) in DMF (1 mL) was added K₂CO₃ (39.56 mg, 286.22 μmol) and MeI (23.76 μL, 381.62 μmol). The reaction mixture was stirred at 80° C. for 12 h. After cooling, the reaction mixture was diluted with H₂O (10 mL), extracted with EtOAc (10 mL×3). The combined organic layers were washed with brine (10 mL×3), dried over Na₂SO₄, filtered, and concentrated to afford ethyl 2-((2S,3R)-3-((tert-butyldimethylsilyl) oxy)-2-(cyclopentyloxy)-3-(3,5-dimethoxy-4-methylphenyl) propyl)-6-(dimethylamino) benzo[d]thiazole-4-carboxylate (55 mg, 87.75% yield), which used for next step without further purification. LC-MS: m/z 657.3 (M+H)⁺.

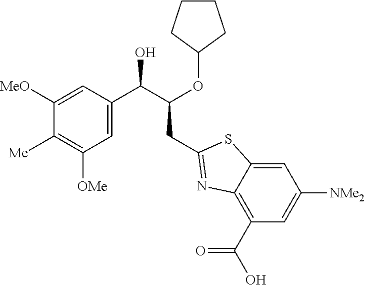 [SMILES: COc1cc([C@@H](O)[C@H](Cc2nc3c(C(=O)O)cc(N(C)C)cc3s2)OC2CCCC2)cc(OC)c1C]

2-((2S,3R)-2-(cyclopentyloxy)-3-(3,5-dimethoxy-4-methylphenyl)-3-hydroxypropyl)-6-(dimethylamino)benzo[d]thiazole-4-carboxylic acid Example A6 (Compound 106) was synthesized according to the procedures described for the preparation of Example A5 (step D to step E in Scheme 5) by using ethyl 2-((2S,3R)-3-((tert-butyldimethylsilyl)oxy)-2-(cyclopentyloxy)-3-(3,5-dimethoxy-4-methylphenyl)propyl)-6-(dimethylamino)benzo[d]thiazole-4-carboxylate (6-2) in step D. LC-MS: m/z 515.3 (M+H)⁺. ¹H NMR (400 MHz, CDCl₃) δ 7.73 (d, J=2.7 Hz, 11H), 7.19 (d, J=2.6 Hz, 1H), 6.58 (s, 2H), 4.94 (d, J=4.3 Hz, 1H), 4.03-3.97 (m, 2H), 3.85 (s, 6H), 3.31-3.24 (m, 1H), 3.14-3.09 (m, 1H), 3.08 (s, 6H), 2.07 (s, 3H), 1.56-1.32 (m, 8H).

### Example A7

### 2-((2S,3R)-2-(cyclopentyloxy)-3-(3,5-dimethoxy-4-methylphenyl)-3-hydroxypropyl)-7-methoxybenzo[d]thiazole-4-carboxylic acid (Compound 107)

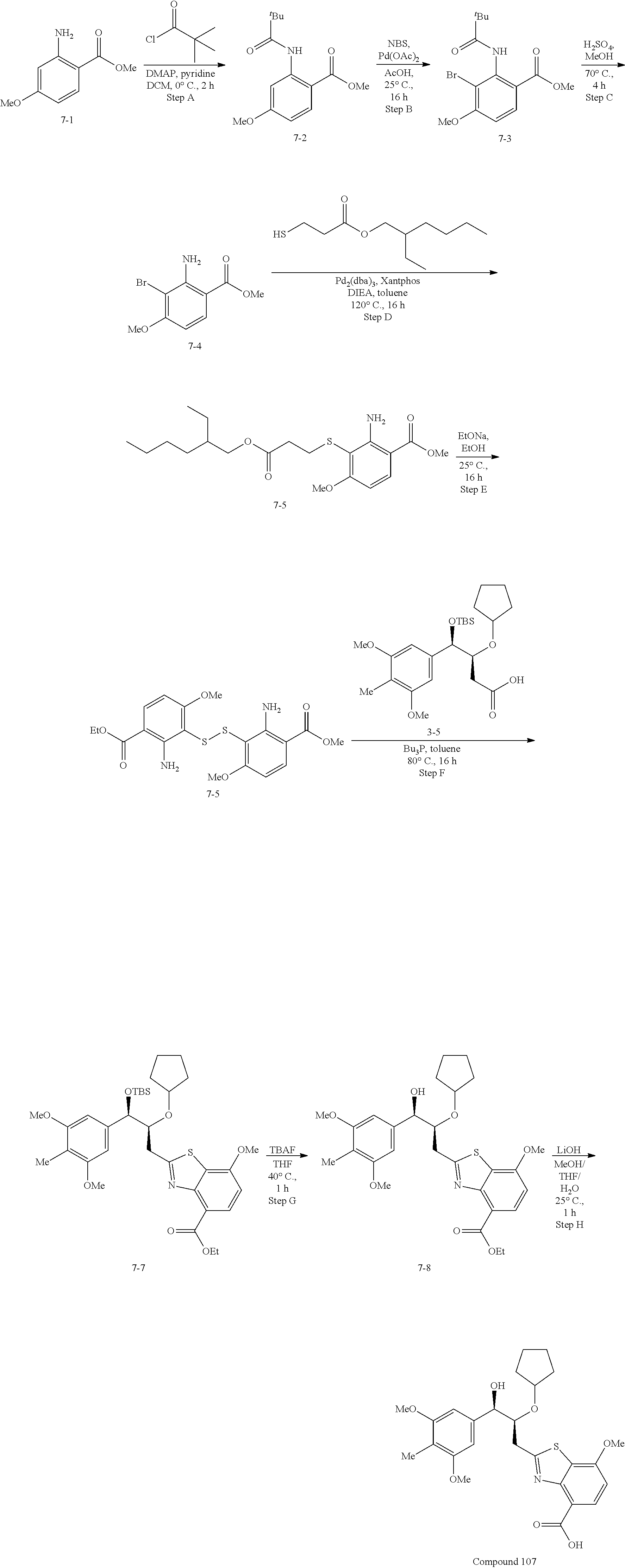

### Step A: methyl 4-methoxy-2-pivalamidobenzoate

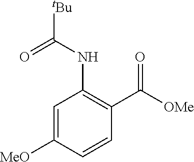 [SMILES: COC(=O)c1ccc(OC)cc1NC(=O)C(C)(C)C]

To a solution of methyl 2-amino-4-methoxybenzoate (25 g, 137.98 mmol) in DCM (200 mL) was added pyridine (22.27 mL, 275.96 mmol) and DMAP (168.57 mg, 1.38 mmol) at 25° C. Then 2,2-dimethylpropanoyl chloride (18.67 mL, 151.78 mmol) was added to the mixture in dropwise at 0° C. The resulting mixture was stirred at 0° C. for 2 h. The reaction mixture was quenched with 1N HCl solution (50 mL) and diluted with DCM (100 mL). The organic layer was separated, washed with 1N HCl solution (40 mL), sat. aq. NaHCO₃ (50 mL) and brine (40 mL). The organic layer was dried over anhydrous Na₂SO₄, filtered and concentrated to give the crude methyl 4-methoxy-2-pivalamidobenzoate (18 g, 60.9% yield), which used for the next step directly without further purification. LC-MS: m/z 266.3 (M+H)⁺.

### Step B: methyl 3-bromo-4-methoxy-2-pivalamidobenzoate

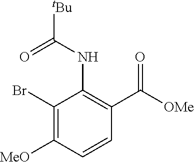 [SMILES: COC(=O)c1ccc(OC)c(Br)c1NC(=O)C(C)(C)C]

To a solution of methyl 2-(2,2-dimethylpropanoylamino)-4-methoxy-benzoate (36 g, 135.69 mmol) in toluene (400 mL) was added Pd(OAc)₂ (6.09 g, 27.14 mmol), NBS (53.13 g, 298.53 mmol) and 4-methylbenzenesulfonic acid (46.73 g, 271.39 mmol). The reaction mixture was stirred at 25° C. for 16 h. Then the reaction mixture was diluted with H₂O (200 mL) and filtered. The filtrate was extracted with EtOAc (150 mL×3). The combined organic layers were washed with brine (100 mL×3), dried over anhydrous Na₂SO₄, filtered and concentrated under reduced pressure. The residue was purified by flash silica gel chromatography (ISCO®; 80 g SepaFlash® Silica Flash Column, Eluent of 0˜15% EtOAc/PE gradient @ 65 mL/min) to give methyl 3-bromo-2-(2,2-dimethylpropanoylamino)-4-methoxy-benzoate (4.8 g, 10.3% yield). LC-MS: m/z 344.2 (M+H)⁺.

### Step C: methyl 2-amino-3-bromo-4-methoxybenzoate

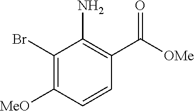 [SMILES: COC(=O)c1ccc(OC)c(Br)c1N]

To a solution of methyl 3-bromo-4-methoxy-2-pivalamidobenzoate (4.8 g, 13.95 mmol) in MeOH (2 mL) was added conc. H₂SO₄ (44.66 g, 24.27 mL) dropwise at 0° C. The reaction mixture was degassed and purged with N₂ for 3 times. The resulting mixture was stirred at 70° C. for 4 h under N₂ atmosphere. After cooling, the reaction was diluted with water (50 mL), extracted with EtOAc (30 mL×3). The combined organic layers were washed with brine (30 mL), dried over anhydrous Na₂SO₄, filtered and concentrated under reduced pressure. The residue was triturated with PE (10 mL) and the suspension isolated via filtration. The filter cake was washed with PE (20 mL) and then dried under reduced pressure to give methyl 2-amino-3-bromo-4-methoxybenzoate (2.4 g, 66.2% yield), which used for the next step directly without further purification. LC-MS: m/z 262.1 (M+H)⁺. ¹H NMR (400 MHz, DMSO-d6) δ 7.82 (d, J=9.0 Hz, 1H), 6.45 (d, J=9.0 Hz, 1H), 3.87 (s, 3H), 3.79 (s, 3H).

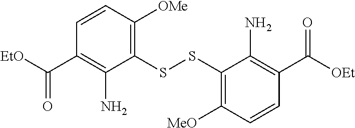 [SMILES: CCOC(=O)c1ccc(OC)c(SSc2c(OC)ccc(C(=O)OCC)c2N)c1N]

Diethyl 3,3′-disulfanediylbis(2-amino-4-methoxybenzoate) (7-6) was synthesized according to the procedures described for the preparation of Example A1 (step C to step D in Scheme 1) by using methyl 2-amino-3-bromo-4-methoxybenzoate (7-4) in step C. LC-MS: m/z 474.9 (M+Na)⁺.

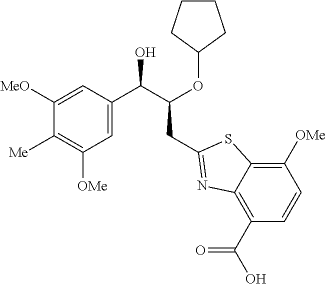 [SMILES: COc1cc([C@@H](O)[C@H](Cc2nc3c(C(=O)O)ccc(OC)c3s2)OC2CCCC2)cc(OC)c1C]

2-((2S,3R)-2-(cyclopentyloxy)-3-(3,5-dimethoxy-4-methylphenyl)-3-hydroxypropyl)-7-methoxybenzo[d]thiazole-4-carboxylic acid (Compound 107) was synthesized according to the procedures described for the preparation of Example A1 (step Q, S and R in Scheme 1) by using diethyl 3,3′-disulfanediylbis(2-amino-4-methoxybenzoate) (7-6) in step D. LC-MS: m/z 502.3 (M+H)⁺. ¹H NMR (400 MHz, CDCl₃) δ 13.36 (br s, 1H), 8.32 (d, J=8.6 Hz, 1H), 6.95 (d, J=8.4 Hz, 1H), 6.58 (s, 2H), 4.95 (d, J=4.3 Hz, 1H), 4.07 (s, 3H), 4.06-3.98 (m, 2H), 3.85 (s, 6H), 3.39 (dd, J=8.3, 15.1 Hz, 1H), 3.21 (dd, J=3.7, 15.1 Hz, 1H), 2.06 (s, 3H), 1.59-1.34 (m, 8H).

### Example A8

### 2-((2S,3R)-2-(cyclopentyloxy)-3-(3,5-dimethoxy-4-methylphenyl)-3-hydroxypropyl)-4-(methylamino)pyrazolo[1,5-a]pyridine-7-carboxylic acid (Compound 108)

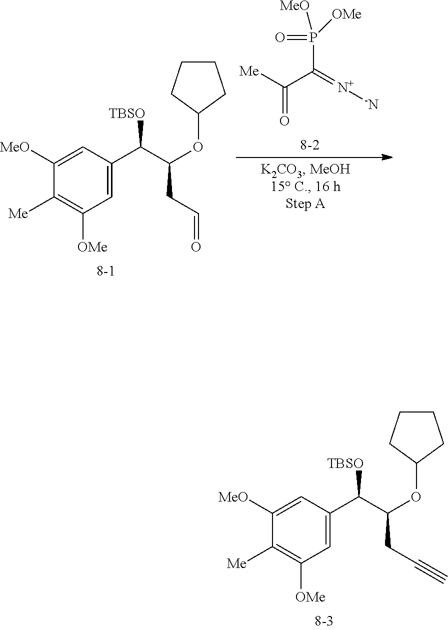

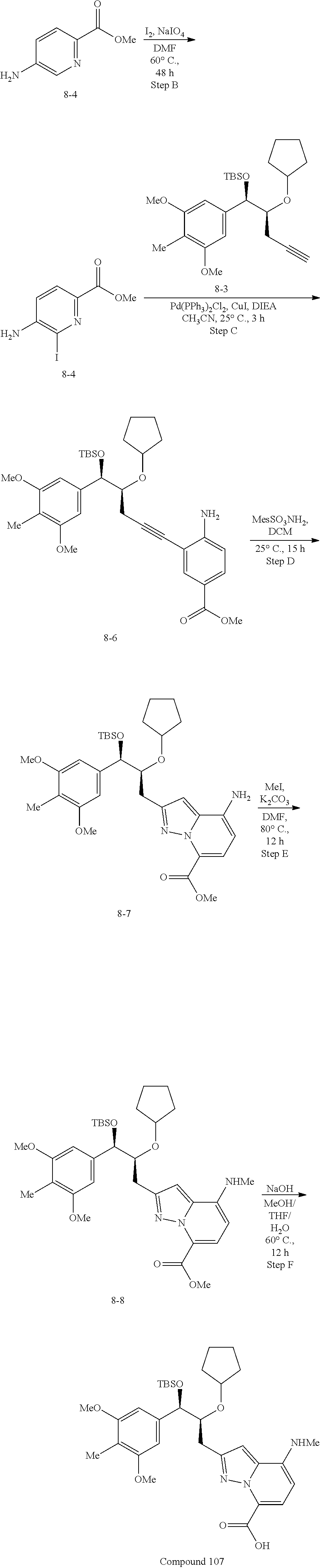

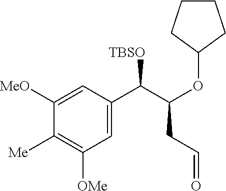 [SMILES: COc1cc([C@@H](O[Si](C)(C)C(C)(C)C)[C@H](CC=O)OC2CCCC2)cc(OC)c1C]

(3S,4R)-4-((tert-butyldimethylsilyl)oxy)-3-(cyclopentyloxy)-4-(3,5-dimethoxy-4-methylphenyl)butanal (8-1) was synthesized according to the procedures described for the preparation of Example A1 (step H to step O in Scheme 1) by using 3,5-dimethoxy-4-methylbenzene-1-carbaldehyde in step H.

### Step A: tert-butyl(((1R,2S)-2-(cyclopentyloxy)-1-(3,5-dimethoxy-4-methylphenyl)pent-4-yn-1-yl)oxy)dimethylsilane

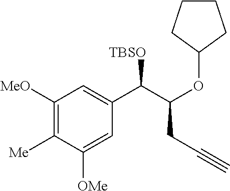 [SMILES: C#CC[C@H](OC1CCCC1)[C@H](O[Si](C)(C)C(C)(C)C)c1cc(OC)c(C)c(OC)c1]

At 0° C., 1-diazo-1-dimethoxyphosphoryl-propan-2-one (3.8 g, 19.8 mmol) was added drop wisely to the mixture of (3S,4R)-4-[tert-butyl(dimethyl)silyl]oxy-3-(cyclopentoxy)-4-(3,5-dimethoxy-4-methyl-phenyl)butanal (7.2 g, 16.5 mmol) and K₂CO₃ (4.56 g, 33.0 mmol) in MeOH (70 mL). The reaction mixture was stirred at 15° C. for 16 h under nitrogen. The mixture was concentrated under vacuum. The residue was purified by flash silica gel chromatography (ISCO®; 220 g SepaFlash® Silica Flash Column, Eluent of 0˜4% EtOAc/PE gradient @ 150 mL/min) to give tert-butyl-[(1R,2S)-2-(cyclopentoxy)-1-(3,5-dimethoxy-4-methyl-phenyl)pent-4-ynoxy]-dimethyl-silane (3.2 g, 44.9% yield). ¹H NMR (400 MHz, CDCl₃) δ 6.54 (s, 2H), 4.59-4.58 (m, 1H), 3.85-3.81 (m, 7H), 3.50-3.46 (m, 1H), 2.48-2.46 (m, 2H), 2.07 (s, 3H), 1.97 (s, 1H), 1.46-1.34 (m, 8H), 0.90 (s, 9H), 0.07 (s, 3H), −0.15 (s, 3H).

### Step B: methyl 5-amino-6-iodopicolinate

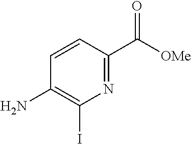 [SMILES: COC(=O)c1ccc(N)c(I)n1]

To a solution of methyl 5-aminopicolinate (10 g, 65.7 mmol) in DMF (80 mL) was added NaIO₄ (5.61 g, 26.2 mmol) and I₂ (13.4 g, 52.7 mmol). The reaction mixture was stirred at 60° C. for 48 h. After cooling, to the reaction mixture was added 10% aq. sodium sulfite solution (100 mL). The resulting mixture was stirred for 10 minutes. The crystals were collected by filtration. The collected crystals were washed with water, dried under reduced pressure to give the methyl 5-amino-6-iodo-pyridine-2-carboxylate (7.84 g, 42.9% yield). ¹H NMR (400 MHz, CDCl₃) δ 7.88 (d, J=8.2 Hz, 1H), 6.95 (d, J=8.2 Hz, 1H), 4.68 (brs, 2H), 3.93 (s, 3H).

### Step C: methyl 5-amino-6-((4S,5R)-5-((tert-butyldimethylsilyl)oxy)-4-(cyclopentyloxy)-5-(3,5-dimethoxy-4-methylphenyl)pent-1-yn-1-yl)picolinate

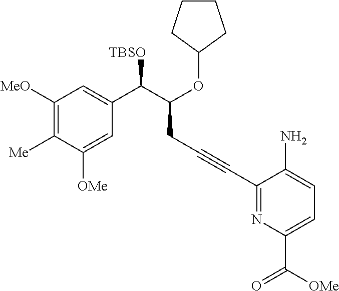 [SMILES: COC(=O)c1ccc(N)c(C#CC[C@H](OC2CCCC2)[C@H](O[Si](C)(C)C(C)(C)C)c2cc(OC)c(C)c(OC)c2)n1]

To a solution of tert-butyl-[(1R,2S)-2-(cyclopentoxy)-1-(3,5-dimethoxy-4-methyl-phenyl)pent-4-ynoxy]-dimethyl-silane (1.71 g, 3.96 mmol) and methyl 5-amino-6-iodo-pyridine-2-carboxylate (1 g, 3.60 mmol) in MeCN (40 mL) was added Pd(PPh₃)₂Cl₂ (252 mg, 360 μmol), CuI (68.5 mg, 360 μmol) and DIEA (3.13 mL, 17.95 mmol). The resulting mixture was stirred at 25° C. for 3 h. The mixture was diluted with water (200 mL), extracted with EtOAc (150 mL×3). The combined organic phase was dried over anhydrous Na₂SO₄, filtered and concentrated to give a residue. The residue was purified by flash silica gel chromatography (ISCO®; 40 g SepaFlash® Silica Flash Column, Eluent of 0˜20% EtOAc/PE gradient @ 100 mL/min) to give methyl 5-amino-6-[(4S,5R)-5-[tert-butyl(dimethyl)silyl]oxy-4-(cyclopentoxy)-5-(3,5-dimethoxy-4-methyl-phenyl)pent-1-ynyl]pyridine-2-carboxylate (1.77 g, 84.2% yield). ¹H NMR (400 MHz, CDCl₃) δ 7.88 (d, J=8.6 Hz, 1H), 6.99 (d, J=8.6 Hz, 1H), 6.55 (s, 2H), 4.75 (s, 2H), 4.65 (d, J=6.0 Hz, 1H), 3.93 (s, 3H), 3.87 (d, J=2.8 Hz, 1H), 3.83 (s, 6H), 3.66-3.57 (m, 1H), 2.91-2.81 (m, 11H), 2.80-2.70 (m, 1H), 2.08 (s, 3H), 1.73-1.63 (m, 2H), 1.52-1.30 (m, 6H), 0.91 (s, 9H), 0.08 (s, 3H),-0.13 (s, 3H).

### Step D: methyl 4-amino-2-((2S,3R)-3-((tert-butyldimethylsilyl)oxy)-2-(cyclopentyloxy)-3-(3,5-dimethoxy-4-methylphenyl)propyl)pyrazolo[1,5-a]pyridine-7-carboxylate

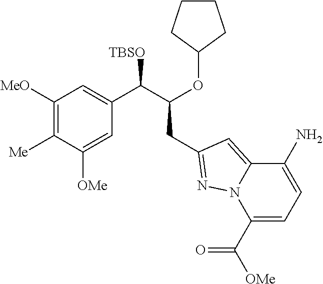 [SMILES: COC(=O)c1ccc(N)c2cc(C[C@H](OC3CCCC3)[C@H](O[Si](C)(C)C(C)(C)C)c3cc(OC)c(C)c(OC)c3)nn12]

The mixture of tert-butyl ((mesitylsulfonyl)oxy)carbamate (946 mg, 3 mmol) and TFA (5 mL) was stirred at 0° C. for 1 h. The mixture was poured into ice-cold water (150 mL), extracted with DCM (20 mL×2). The combined organic layers were dried over anhydrous Na₂SO₄, filtered, and the filtrate was added dropwise to a solution of methyl 5-amino-6-[(4S,5R)-5-[tert-butyl(dimethyl)silyl]oxy-4-(cyclopentoxy)-5-(3,5-dimethoxy-4-methyl-phenyl)pent-1-ynyl]pyridine-2-carboxylate (0.96 g, 1.65 mmol) in DCM (20 mL) at 25° C. The resulting mixture was stirred at 25° C. for 15 h. The reaction mixture was quenched with sat. aq. NaHCO₃ (150 mL). The organic layer was separated, dried over anhydrous Na₂SO₄, filtered and concentrated to give a residue. The residue was purified by flash silica gel chromatography (ISCO®; 20 g SepaFlash® Silica Flash Column, Eluent of 0˜20-40% EtOAc/PE gradient @ 80 mL/min) to give methyl 4-amino-2-[(2S,3R)-3-[tert-butyl(dimethyl)silyl]oxy-2-(cyclopentoxy)-3-(3,5-dimethoxy-4-methyl-phenyl)propyl]pyrazolo[1,5-a]pyridine-7-carboxylate (168 mg, 17.1% yield). LC-MS: m/z 598.8 (M+H)⁺.

### Step E: methyl 2-((2S,3R)-3-((tert-butyldimethylsilyl)oxy)-2-(cyclopentyloxy)-3-(3,5-dimethoxy-4-methylphenyl)propyl)-4-(methylamino)pyrazolo[1,5-a]pyridine-7-carboxylate

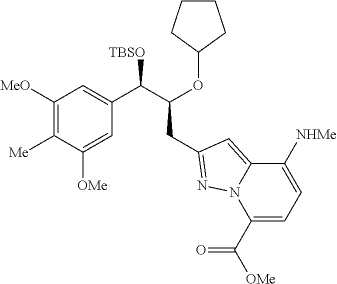 [SMILES: CNc1ccc(C(=O)OC)n2nc(C[C@H](OC3CCCC3)[C@H](O[Si](C)(C)C(C)(C)C)c3cc(OC)c(C)c(OC)c3)cc12]

To a solution of methyl 4-amino-2-[(2S,3R)-3-[tert-butyl(dimethyl)silyl]oxy-2-(cyclopentoxy)-3-(3,5-dimethoxy-4-methyl-phenyl)propyl]pyrazolo[1,5-a]pyridine-7-carboxylate (84 mg, 141 μmol) in DMF (2 mL) was added K₂CO₃ (58.3 mg, 422 μmol) and MeI (70.0 μL, 1.12 mmol). The reaction mixture was stirred at 80° C. for 12 h. After cooling, the mixture was diluted with water (30 mL), extracted with EtOAc (30 mL×4). The combined organic layers were dried over anhydrous Na₂SO₄, filtered and concentrated to give a residue. The residue was purified by flash silica gel chromatography (ISCO®; 4 g SepaFlash® Silica Flash Column. Eluent of 0˜20% EtOAc/PE gradient @& 100 mL/min) to give methyl 2-[(2S,3R)-3-[tert-butyl(dimethyl)silyl]oxy-2-(cyclopentoxy)-3-(3,5-dimethoxy-4-methyl-phenyl)propyl]-4-(methylamino)pyrazolo[1,5-a]pyridine-7-carboxylate (20 mg, 23.3% yield). LC-MS: m/z 634.0 (M+Na)⁺.

### Step F: 2-((2S,3R)-2-(cyclopentyloxy)-3-(3,5-dimethoxy-4-methylphenyl)-3-hydroxypropyl)-4-(methylamino)pyrazolo[1,5-a]pyridine-7-carboxylic acid

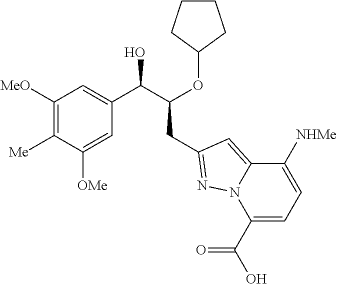 [SMILES: CNc1ccc(C(=O)O)n2nc(C[C@H](OC3CCCC3)[C@H](O)c3cc(OC)c(C)c(OC)c3)cc12]

To a solution of methyl 2-[(2S,3R)-3-[tert-butyl(dimethyl)silyl]oxy-2-(cyclopentoxy)-3-(3,5-dimethoxy-4-methyl-phenyl)propyl]-4-(methylamino)pyrazolo[1,5-a]pyridine-7-carboxylate (20 mg, 32.7 μmol) in THF (2 mL). MeOH (5 mL) and H₂O (2 mL) was added NaOH (261 mg, 6.54 mmol). The reaction mixture was stirred at 60° C. for 12 h. The solvent was removed under vacuum. The residue was diluted with water (5 mL), acidified with 6N HCl to pH=4-5. The mixture was extracted with EtOAc (30 mL×3). The organic layers were dried over Na₂SO₄, filtered and concentrated under vacuum to give a residue. The residue was purified by prep. HPLC (Column: Kromasil 100-5-C18; Mobile Phase A: Water (0.1% HCOOH), Mobile Phase B: CH₃CN; Gradient: 50% B to 80% B in 8 min) to give 2-[(2S,3R)-2-(cyclopentoxy)-3-(3,5-dimethoxy-4-methyl-phenyl)-3-hydroxy-propyl]-4-(methylamino)pyrazolo[1,5-a]pyridine-7-carboxylic acid (2.33 mg, 14.52% yield). LC-MS: m/z 484.3 (M+H)⁺. ¹H NMR (400 MHz, CD₃OD) δ 7.76 (d, J=8.2 Hz, 1H), 6.71 (s, 1H), 6.64 (s, 2H), 6.24 (d, J=8.2 Hz, 1H), 4.61-4.58 (m, 2H), 3.87-3.74 (m, 8H), 3.22-3.14 (m, 1H), 3.07-3.01 (m, 1H), 3.00 (s, 3H), 1.99 (s, 3H), 1.46-1.24 (m, 8H).

The compounds in Table 1 can be or were synthesized using a similar procedure described in the Examples above using the appropriate starting materials. Data for certain compounds is shown in the Table below. Purification conditions for certain compounds is as follows.

Prep. HPLC separation conditions for compound 102: Column: Welch Xtimate C18 150*30 mm*5 μm; Mobile Phase A: Water (0.1% HCOOH), Mobile Phase B: CH₃CN; Gradient: 57% B to 87% B in 7 min.

Prep. HPLC separation conditions for compound 103: Column: Kromasil 100-5-C18; Mobile Phase A: Water (0.10% HCOOH), Mobile Phase B: CH₃CN; Gradient: 50% B to 80% B in 10 min.

Prep. HPLC separation conditions for compound 104: Column: Welch Xtimate C18 150*30 mm*5 μm; Mobile Phase A: Water (0.01% NH₃H₂O+10 mM NH₄HCO₃), Mobile Phase B: CH₃CN; Gradient: 20% B to 50% B in 9 min.

Prep. HPLC separation conditions for compound 106: Column: Kromasil 100-5-C18; Mobile Phase A: Water (0.1% HCOOH), Mobile Phase B: CH₃CN; Gradient: 50% B to 90% B in 10 min.

Prep. HPLC separation conditions for compound 107: Column: Kromasil 100-5-C18; Mobile Phase A: Water (0.1% HCOOH). Mobile Phase B: CH₃CN; Gradient: 50% B to 85% B in 10 min.

Prep. HPLC separation conditions for compound 109: Column: Kromasil 100-5-C18; Mobile Phase A: Water (0.1% CF3COOH), Mobile Phase B: CH₃CN; Gradient: 50% B to 90% B in 10 min.

Prep. HPLC separation conditions for compound 110: Column: Kromasil 100-5-C18; Mobile Phase A: Water (0.1% HCOOH), Mobile Phase B: CH₃CN; Gradient: 55% B to 95% B in 9 min.

Prep. HPLC separation conditions for compound 111: Column: Boston Green ODS (150*30 mm*5 μm); Mobile Phase A: Water (0.225% HCOOH), Mobile Phase B: CH₃CN; Gradient: 60% B to 90% B in 8 min.

Prep. HPLC separation conditions for compound 112: Column: Phenomenex Gemini-NX C18 75*30 mm*3 μm; Mobile Phase A: Water (0.225% HCOOH), Mobile Phase B: CH₃CN; Gradient: 63% B to 93% B in 8 min.

Prep. HPLC separation conditions for compound 113: Column: Kromasil 100-5-C18; Mobile Phase A: Water (0.1% HCOOH), Mobile Phase B: CH₃CN; Gradient: 55% B to 100% B in 10 min.

Prep. HPLC separation conditions for compound 114: Column: DAICEL CHIRALPAK IF (250 mm*30 mm*10 μm); Mobile Phase A: Water (0.1% NH₃H₂O), Mobile Phase B: EtOH; Gradient: 20% B to 20% B in 8 min.

| Compound | Structure | LC-MS: m/z |
|---|---|---|
| 101 | 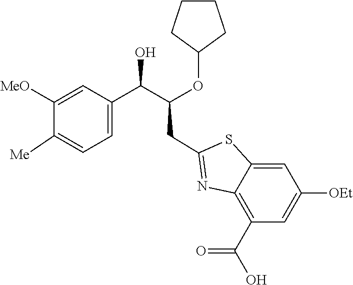 [SMILES: CCOc1cc(C(=O)O)c2nc(C[C@H](OC3CCCC3)[C@H](O)c3ccc(C)c(OC)c3)sc2c1] | 486.1 (M + H)⁺ |
| 102 | 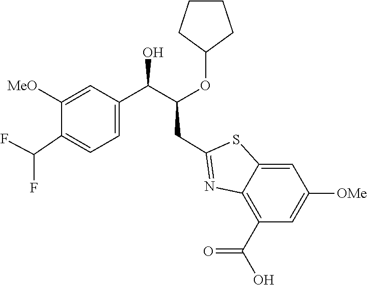 [SMILES: COc1cc(C(=O)O)c2nc(C[C@H](OC3CCCC3)[C@H](O)c3ccc(C(F)F)c(OC)c3)sc2c1] | 508.0 (M + H)⁺ |
| 103 | 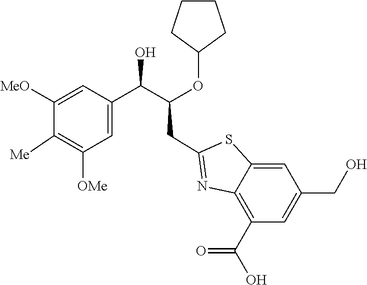 [SMILES: COc1cc([C@@H](O)[C@H](Cc2nc3c(C(=O)O)cc(CO)cc3s2)OC2CCCC2)cc(OC)c1C] | 502.3 (M + H)⁺ |
| 104 | 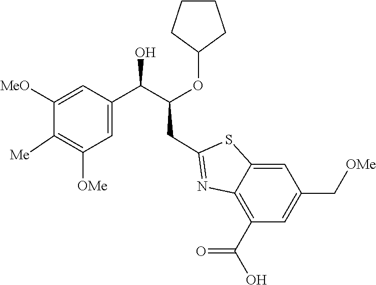 [SMILES: COCc1cc(C(=O)O)c2nc(C[C@H](OC3CCCC3)[C@H](O)c3cc(OC)c(C)c(OC)c3)sc2c1] | 538.1 (M + Na)⁺ |
| 105 | 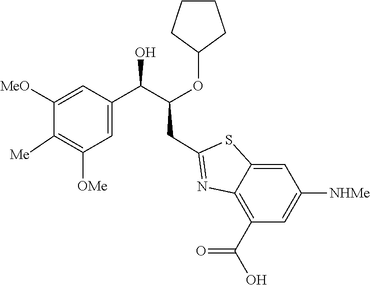 [SMILES: CNc1cc(C(=O)O)c2nc(C[C@H](OC3CCCC3)[C@H](O)c3cc(OC)c(C)c(OC)c3)sc2c1] | 501.3 (M + H)⁺ |
| 106 | 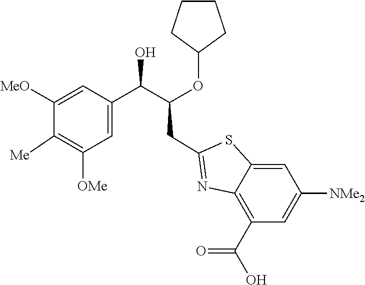 [SMILES: COc1cc([C@@H](O)[C@H](Cc2nc3c(C(=O)O)cc(N(C)C)cc3s2)OC2CCCC2)cc(OC)c1C] | 515.3 (M + H)⁺ |
| 107 | 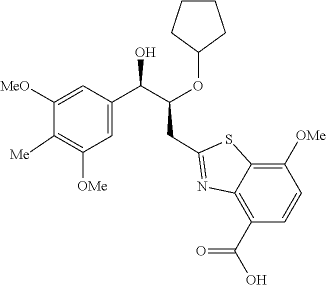 [SMILES: COc1cc([C@@H](O)[C@H](Cc2nc3c(C(=O)O)ccc(OC)c3s2)OC2CCCC2)cc(OC)c1C] | 502.3 (M + H)⁺ |
| 108 | 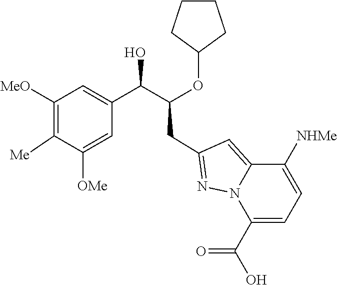 [SMILES: CNc1ccc(C(=O)O)n2nc(C[C@H](OC3CCCC3)[C@H](O)c3cc(OC)c(C)c(OC)c3)cc12] | 484.3 (M + H)⁺ |
| 109 | 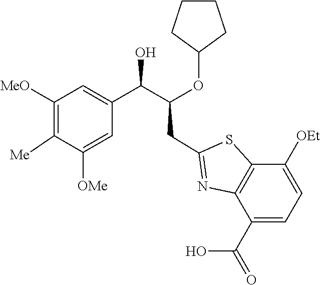 [SMILES: CCOc1ccc(C(=O)O)c2nc(C[C@H](OC3CCCC3)[C@H](O)c3cc(OC)c(C)c(OC)c3)sc12] | 516.4 (M + H)⁺ |
| 110 | 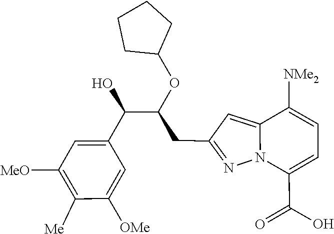 [SMILES: COc1cc([C@@H](O)[C@H](Cc2cc3c(N(C)C)ccc(C(=O)O)n3n2)OC2CCCC2)cc(OC)c1C] | 498.4 (M + H)⁺ |
| 111 | 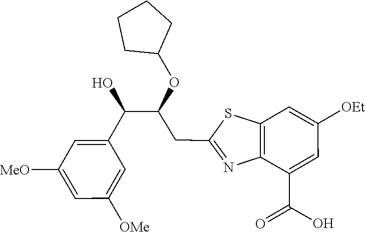 [SMILES: CCOc1cc(C(=O)O)c2nc(C[C@H](OC3CCCC3)[C@H](O)c3cc(OC)cc(OC)c3)sc2c1] | 502.1 (M + H)⁺ |
| 112 | 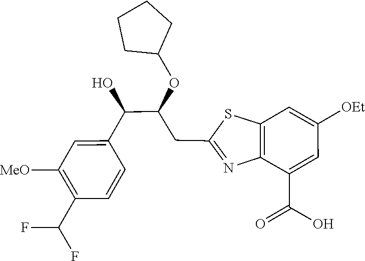 [SMILES: CCOc1cc(C(=O)O)c2nc(C[C@H](OC3CCCC3)[C@H](O)c3ccc(C(F)F)c(OC)c3)sc2c1] | 522.0 (M + H)⁺ |
| 113 | 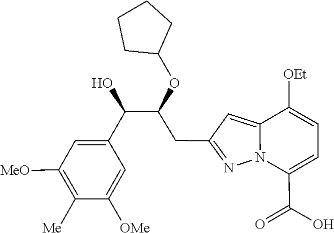 [SMILES: CCOc1ccc(C(=O)O)n2nc(C[C@H](OC3CCCC3)[C@H](O)c3cc(OC)c(C)c(OC)c3)cc12] | 499.4 (M + H)⁺ |
| 114 | 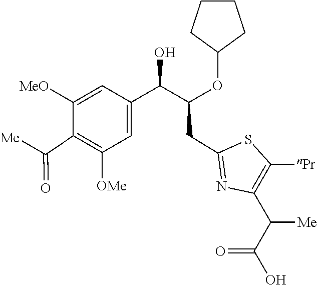 | 520.0 (M + H)⁺ |

### Biological Assays

### In Vitro LPA1 Functional Antagonist Assay

CHO-K1 cells overexpressing human LPA1 are seeded in a total volume of 20 μL into black-walled, clear-bottom, Poly-D-lysine coated 384-well microplates and incubated at 37° C. for the appropriate time prior to testing. Assays are performed in 1×Dye Loading Buffer consisting of 1× Dye, 1× Additive A and 2.5 mM Probenecid in HBSS/20 mM Hepes. Probenicid is prepared fresh. Cells are loaded with dye prior to testing. Media is aspirated from cells and replaced with 20 μL Dye Loading Buffer. Cells are incubated for 30-60 minutes at 37° C. After dye loading, cells are removed from the incubator and 10 μL 3× test compound is added. Cells are incubated for 30 minutes at room temperature in the dark to equilibrate plate temperature followed by Oleoyl LPA challenge at the 0.018 μM. Compound antagonist activity is measured on a FLIPR Tetra (MDS). Calcium mobilization is monitored for 2 minutes and 10 μL Oleoyl LPA in HBSS/20 mM Hepes is added to the cells 5 seconds into the assay. Compound activity is analyzed using CBIS data analysis suite (ChemInnovation, CA). Percentage inhibition is calculated using the following formula:

% Inhibition=100%×(1−(mean RFU of test sample−mean RFU of vehicle control)/(mean RFU of LPA control−mean RFU of vehicle control)).

### In Vitro LPA1 Calcium Flux Antagonist Assay

CHO-K1 cells overexpressing human LPA1 and G15a were seeded at a total volume of 20 μL (15000 cells/well) into Matrigel pre-coated 384-well plates (corning −3764) and incubated at 37° C. After overnight incubation, the cells were serum starved for 4 h. Assays were performed in dye loading buffer containing 1×Fluo-8 AM (AAT Bioquest, 21080) and 2.5 mM probenecid (Thermo Fisher, 36400) in HBSS/20 mM Hepes. After cell starvation, the medium was replaced with 20 μL of dye loading buffer and incubated at 37° C. for 30 min. Then 5 μL of 5× compound titrated in dye loading buffer was added to the cells and incubated for 30 min followed by LPA challenge at EC80. Calcium mobilization was measured on a FLIPR Tetra (MDS). For LPA EC80 determination, starved cells were incubated with 20 μL of dye loading buffer for 1 h, then 5 μL of 5×LPA titrated in dye loading buffer was added to the cells. Calcium signals induced by LPA was monitors on a FLIPR.

Percentage inhibition is calculated using the following formula:

$$\% Inhibition = 100 \% \times ( 1 - ( mean RFU of test sample - mean RFU of DMSO ) / ( mean RFU of LPA control - mean RFU of DMSO ) ) .$$

Table B1 shows the biological activity of compounds in in vitro LPA1 Calcium flux antagonist assay. Activity of the tested compounds is provided in Table B1 below as follows: +++=IC₅₀<10 nM; ++=IC₅₀ 10 nM-100 nM; +=IC₅₀>100 nM.

TABLE B1

| Compound | Activity |
|---|---|
| 101 | +++ |
| 102 | +++ |
| 103 | ++ |
| 104 | +++ |
| 105 | +++ |
| 106 | +++ |
| 107 | +++ |
| 108 | ++ |
| 109 | +++ |
| 110 | + |
| 111 | ++ |
| 112 | ++ |
| 113 | ++ |
| 114 | ++ |

### In Vivo Studies

### Pharmacokinetics Measurements in Mice

The PK properties of selected compounds were measured in CD1 female mice following single oral (5 mg/kg) administration.

Compounds 103 and 104 were prepared in a 1 mg/mL solution of 10% Solutol HS15 and 90%, saline for oral administrations at 5 mL/kg, respectively, and administered to 3 mice/3 groups. After dosing, blood collection was performed by dorsal metatarsal vein sampling at 0.25, 0.5, 1, 2, 4, 6, 8, 24 h, followed by centrifugation to obtain plasma. Samples were stored frozen at −80° C. prior to compound extraction and LC-MS/MS analysis. Pharmacokinetic parameters of compounds 103 and 104 in mice were calculated by standard noncompartmental modeling from the systemic plasma concentration-time profile.

Table B2 shows the mean pharmacokinetic parameters of compounds 103 and 104 in mice (CD1: female) determined by the non-compartmental model.

TABLE B2

Compound

| PK parameter | 103 | 104 |
|---|---|---|
| Tₘₐₓ (h) | 0.500 | 0.250 |
| Cₘₐₓ (ng/mL) | 463 | 3837 |
| AUCₗₐₛₜ (h*ng/mL) | 1643 | 6947 |

### Pharmacokinetics Measurements in Rats

The PK properties of selected compounds were measured in SD Male rats following single oral (5 mg/kg) and Intravenous Administration (1 mg/kg).

Compounds 103 and 104 were prepared in a 1 mg/mL solution of 10% Solutol HS15 and 90% saline for oral administrations at 5 mL/kg and 0.2 mg/mL solution of 10% Solutol HS15 and 90% saline for intravenous administrations at 1 mL/kg, respectively, and administered to 3 rats per group. After dosing, blood collection was performed by Jugular vein sampling (by cannula) at 0.25, 0.5, 1, 2, 4, 6, 8, 24 h, and 0.083, 0.25, 0.5, 1, 2, 4, 8, 24 h, respectively, followed by centrifugation to obtain plasma. Samples were stored frozen at −80° C. prior to compound extraction and LC-MS/MS analysis. Pharmacokinetic parameters of compounds 103 and 104 in rats were calculated by standard noncompartmental modeling from the systemic plasma concentration-time profile.

Table B3 shows the mean pharmacokinetic parameters of compounds 103 and 104 in rats (SD; male) determined by the non-compartmental model.

TABLE B3

| | Compound | | | |
|---|---|---|---|---|
| PK parameter | 103 | | 104 | |
| Route | IV | PO | IV | PO |
| Cl_obs (mL/min/kg) | 12.9 | ND | 8.8 | ND |
| Tₘₐₓ (h) | ND | 0.25 | ND | 0.25 |
| Cₘₐₓ (ng/mL) | ND | 1493 | N.D | 1519 |
| AUCₗₐₛₜ (h*ng/mL) | 1334 | 1794 | 1899 | 2894 |
| F(%) | ND | 27.3 | ND | 30.3 |

1. A compound selected from: 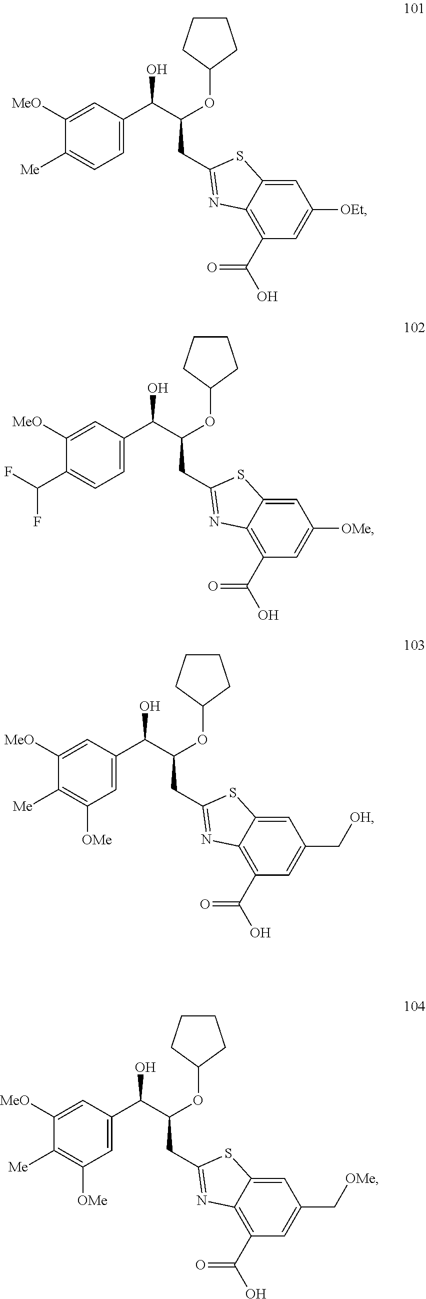 [SMILES: CCOc1cc(C(=O)O)c2nc(C[C@H](OC3CCCC3)[C@H](O)c3ccc(C)c(OC)c3)sc2c1 ; COc1cc(C(=O)O)c2nc(C[C@H](OC3CCCC3)[C@H](O)c3ccc(C(F)F)c(OC)c3)sc2c1 ; COc1cc([C@@H](O)[C@H](Cc2nc3c(C(=O)O)cc(CO)cc3s2)OC2CCCC2)cc(OC)c1C ; COCc1cc(C(=O)O)c2nc(C[C@H](OC3CCCC3)[C@H](O)c3cc(OC)c(C)c(OC)c3)sc2c1] 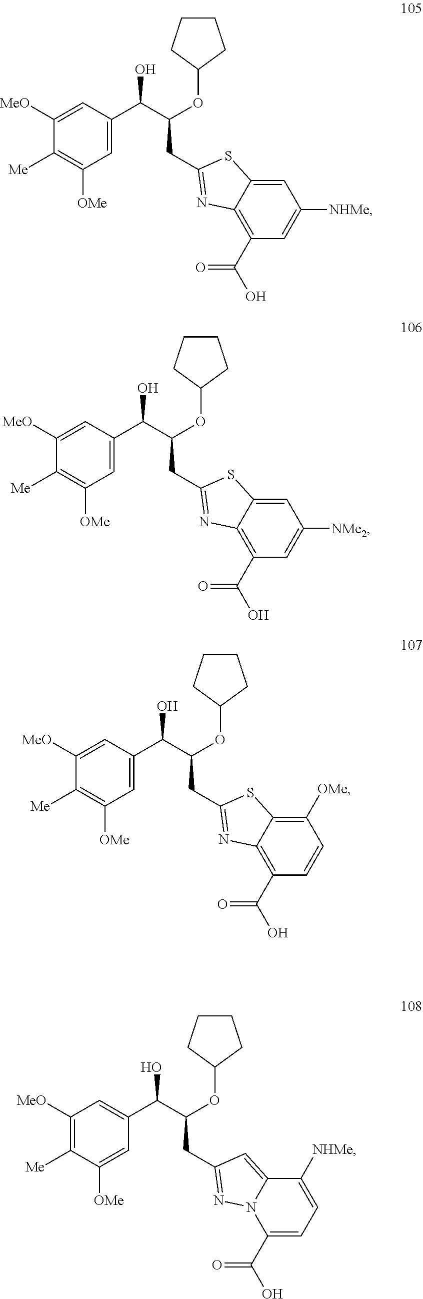 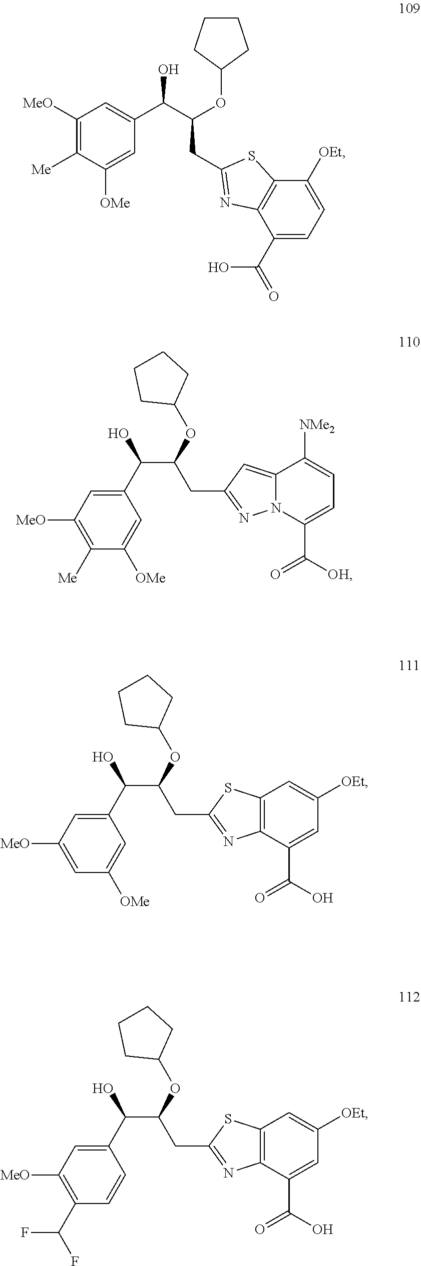 [SMILES: CCOc1ccc(C(=O)O)c2nc(C[C@H](OC3CCCC3)[C@H](O)c3cc(OC)c(C)c(OC)c3)sc12 ; COc1cc([C@@H](O)[C@H](Cc2cc3c(N(C)C)ccc(C(=O)O)n3n2)OC2CCCC2)cc(OC)c1C ; CCOc1cc(C(=O)O)c2nc(C[C@H](OC3CCCC3)[C@H](O)c3cc(OC)cc(OC)c3)sc2c1 ; CCOc1cc(C(=O)O)c2nc(C[C@H](OC3CCCC3)[C@H](O)c3ccc(C(F)F)c(OC)c3)sc2c1] 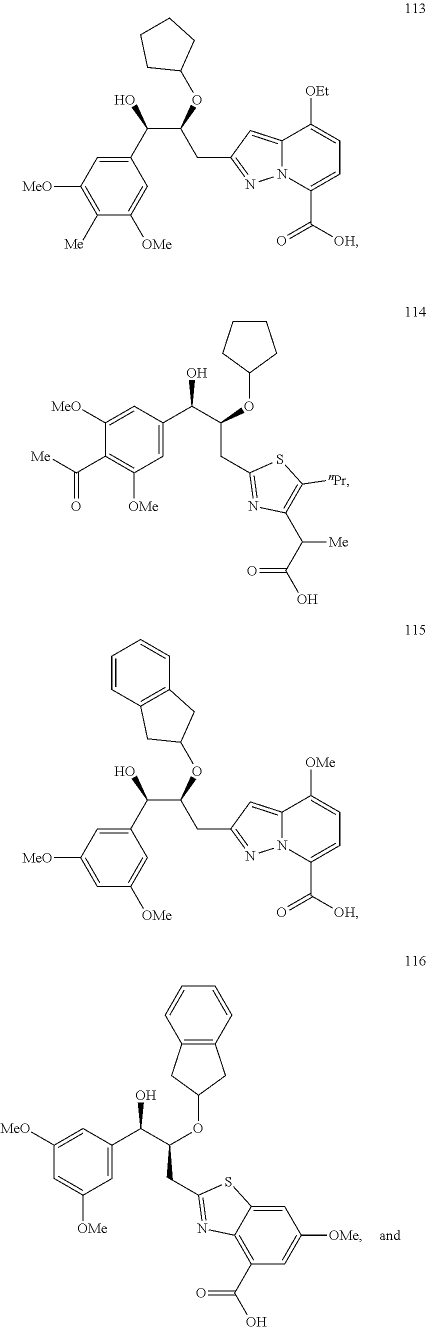 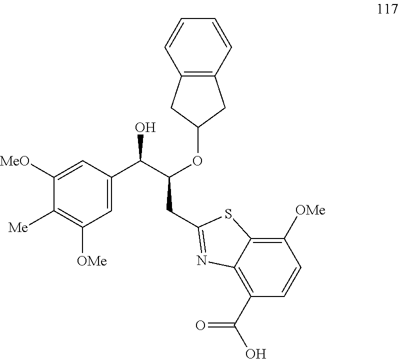 [SMILES: COc1cc([C@@H](O)[C@H](Cc2nc3c(C(=O)O)ccc(OC)c3s2)OC2Cc3ccccc3C2)cc(OC)c1C]
or a pharmaceutically acceptable salt or solvate thereof.

2. A compound selected from 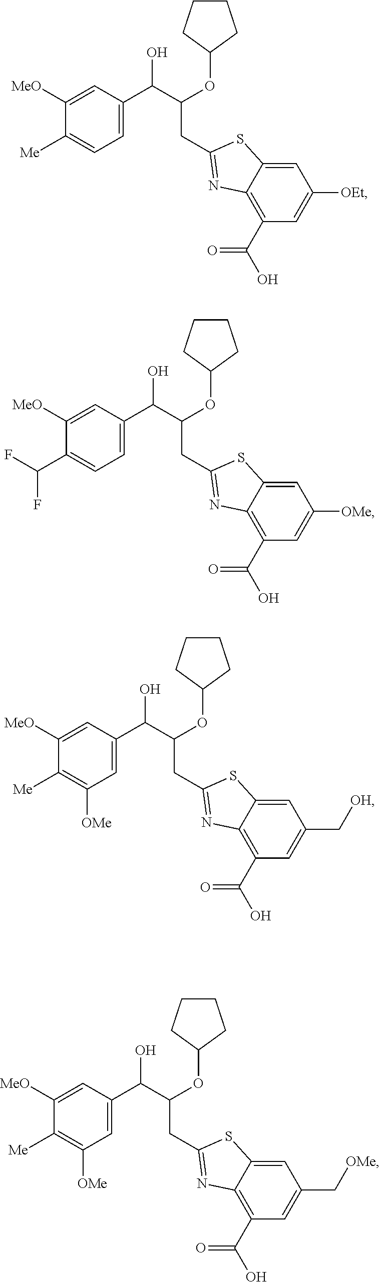 [SMILES: CCOc1cc(C(=O)O)c2nc(CC(OC3CCCC3)C(O)c3ccc(C)c(OC)c3)sc2c1 ; COc1cc(C(=O)O)c2nc(CC(OC3CCCC3)C(O)c3ccc(C(F)F)c(OC)c3)sc2c1 ; COc1cc(C(O)C(Cc2nc3c(C(=O)O)cc(CO)cc3s2)OC2CCCC2)cc(OC)c1C ; COCc1cc(C(=O)O)c2nc(CC(OC3CCCC3)C(O)c3cc(OC)c(C)c(OC)c3)sc2c1] 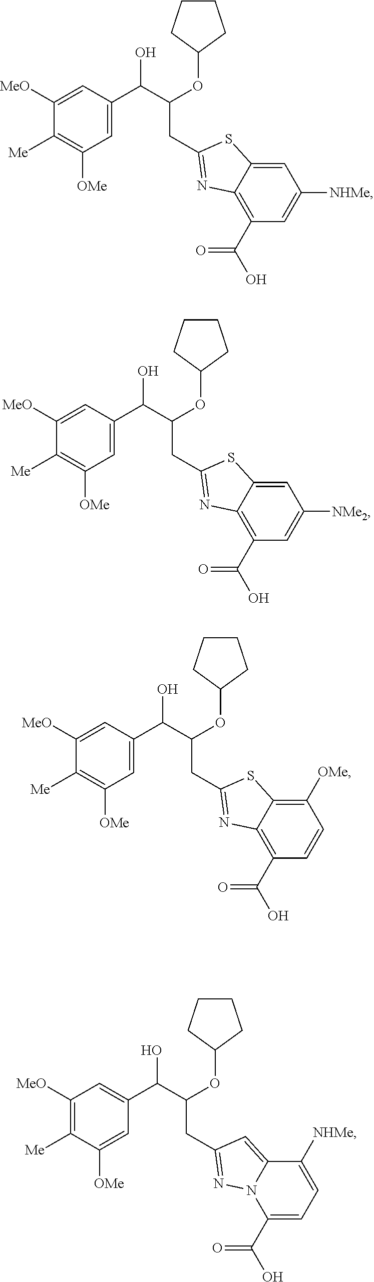 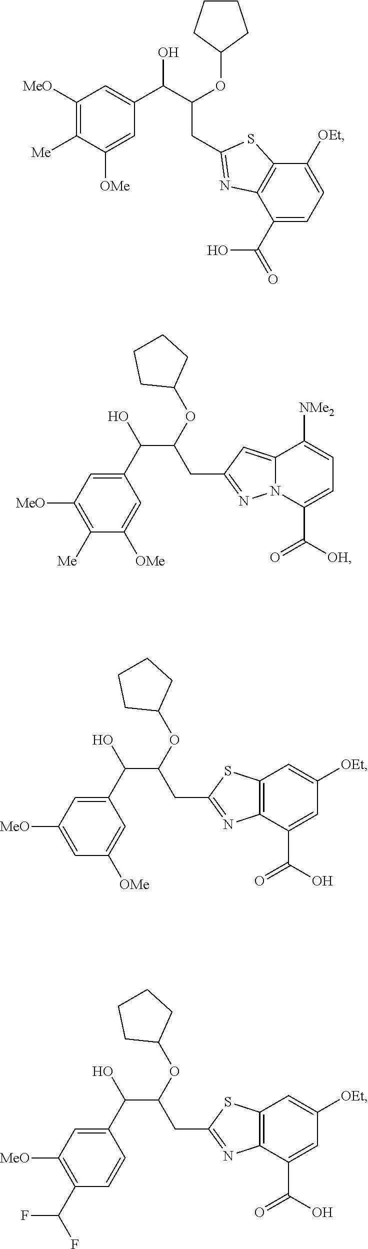 [SMILES: CCOc1ccc(C(=O)O)c2nc(CC(OC3CCCC3)C(O)c3cc(OC)c(C)c(OC)c3)sc12 ; COc1cc(C(O)C(Cc2cc3c(N(C)C)ccc(C(=O)O)n3n2)OC2CCCC2)cc(OC)c1C ; CCOc1cc(C(=O)O)c2nc(CC(OC3CCCC3)C(O)c3cc(OC)cc(OC)c3)sc2c1 ; CCOc1cc(C(=O)O)c2nc(CC(OC3CCCC3)C(O)c3ccc(C(F)F)c(OC)c3)sc2c1] 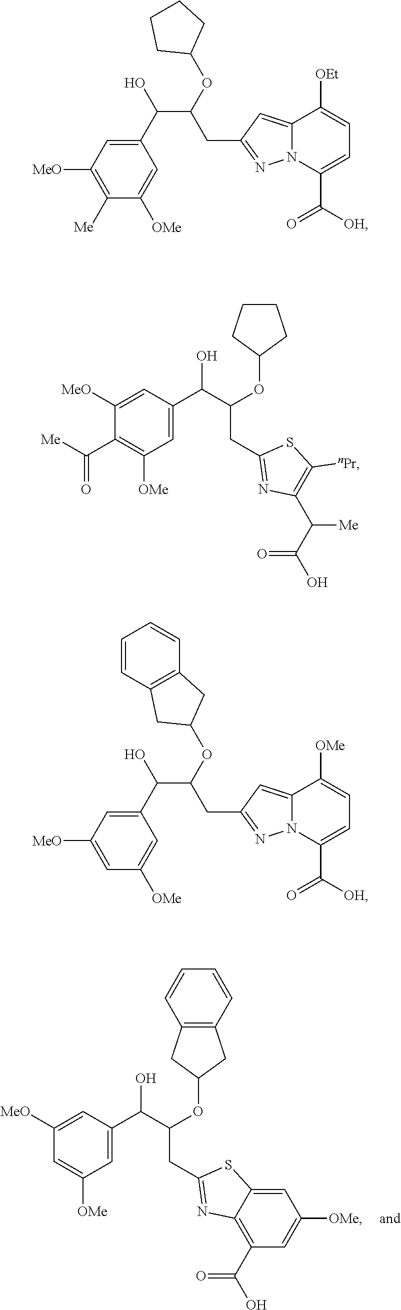 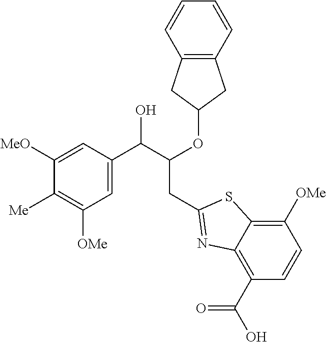 [SMILES: COc1cc(C(O)C(Cc2nc3c(C(=O)O)ccc(OC)c3s2)OC2Cc3ccccc3C2)cc(OC)c1C]
or a pharmaceutically acceptable salt or solvate thereof.

3. A pharmaceutical composition comprising a compound of claim 1, or a pharmaceutically acceptable salt or solvate thereof, and a pharmaceutically acceptable carrier.

4. A method for treating a LPA associated disease, disorder, or condition, the method comprising administering to a patient in need thereof an effective amount of a compound of claim 1, or a pharmaceutically acceptable salt or solvate thereof, or the pharmaceutical composition according to claim 3.

5. The method of claim 4, wherein the LPA-associated disease, disorder, or condition, is a LPA₁-associated disease.

6. The method of claim 4, wherein the LPA-associated disease, disorder, or condition, is fibrosis, transplant rejection, cancer, osteoporosis, or an inflammatory disorder.

7. The method of claim 6, wherein the fibrosis is pulmonary fibrosis, liver fibrosis, renal fibrosis, cardiac fibrosis, dermal fibrosis, ocular fibrosis, or pancreatic fibrosis.

8. The method of claim 6, wherein the cancer is of the bladder, blood, bone, brain, breast, central nervous system, cervix, colon, endometrium, esophagus, gall bladder, genitalia, genitourinary tract, head, kidney, larynx, liver, lung, muscle tissue, neck, oral or nasal mucosa, ovary, pancreas, prostate, skin, spleen, small intestine, large intestine, stomach, testicle, or thyroid.

9. The method of claim 4, wherein the LPA-associated disease, disorder, or condition, is idiopathic pulmonary fibrosis (IPF), non-alcoholic steatohepatitis (NASH), non-alcoholic fatty liver disease (NAFLD), chronic kidney disease, diabetic kidney disease, systemic sclerosis, COVID-19, chronic obstructive pulmonary disease (COPD), neuroinflammation, or multiple sclerosis.
